(19) United States
(12) Patent Application Publication
(10) Pub. No.: US 2024/0150507 A1
(43) Pub. Date: May 9, 2024

(54) PROGRAMMABLE POLYMER DROPLETS AND ASSOCIATED USES

(71) Applicant: ETH ZURICH, Zurich (CH)

(72) Inventors: Paolo AROSIO, Zurich (CH); Marie KOPP, Zurich (CH); Umberto CAPASSO PALMIERO, Zurich (CH); Carolina PAGANINI, Zurich (CH)

(21) Appl. No.: 18/256,145

(22) PCT Filed: Dec. 6, 2021
(86) PCT No.: PCT/EP2021/084452

§ 371 Date: Jun. 6, 2023

(30) Foreign Application Priority Data
Dec. 7, 2020 (EP) 20212188.5

(51) Int. Cl. C08F 220/38

(52) U.S. Cl. CPC C08F 220/387; C08F 2438/03

(57) ABSTRACT

The present invention pertains to liquid droplets including a polymer according to Formula (I) and to their use in an aqueous medium for the uptake of compounds from outside the droplets into the droplets for separation, storage and/or reaction of compounds inside the droplets, as well as associated methods of production of the polymer.

### CROSS-REFERENCE TO RELATED APPLICATIONS

The present application is a National Stage entry of International Patent Application No. PCT/EP2021/084452, filed 6 Dec. 2021 and published as International Patent Application Publication No. WO 2022/122675 A1, which claims priority to, and the benefit of, European Patent Application No. 20212188.5, filed 7 Dec. 2020, each of which is incorporated by reference herein in its entirety for all purposes.

### TECHNICAL FIELD

The present invention pertains to liquid droplets comprising a polymer according to Formula (I) and to their use in an aqueous medium for the uptake of compounds from outside the droplets into the droplets for separation, storage and/or reaction of compounds inside the droplets.

### BACKGROUND

The bioseparation and detection of biomolecules in complex media can require laborious and expensive procedures. The selective capture of biomolecules in complex media is an important aspect both in the development of biomedical detection assays and in the purification of biopharmaceuticals. The biopurification, bioseparation and detection of analytes is commonly performed by either using solid resins or thermoresponsive polymers functionalized with targeting agents. Relevant examples include PNIPAM, peptide polymers and condensed phases obtained via simple or complex coacervation (see, e.g., I. N. Savina, et al., Chapter 15, Smart Polymers and their Applications (Second Edition), Woodhead Publishing, 2019, Pages 533-565; Rahul D. Sheth et al., Journal of Biotechnology, 192, Part A, 2014, 11-19; N. Shimada et al., Biomacromolecules 2013, 14, 5, 1452-1457; J. van Lente et al., Biomacromolecules 2019, 20, 10, 3696-3703). However, these solid or solid-like materials do not have the ability to renew their interface and, thus, present lower concentration efficacies. In addition, they encode multiple interactions which are not able to completely exclude solutes from complex mixture. For this reason, they require multiple washing steps to remove all the unspecific solutes that are retained during the separation process. This aspect can challenge their application for the detection of pathological biomarkers and the purification of biomolecules.

It is the objective of the present invention to provide materials with utility in purification, separation and detection of compounds in liquid environments.

### SUMMARY

In a first aspect, the present invention is directed to a use of liquid droplets in an aqueous medium for the uptake of compounds, optionally the controlled uptake of compounds, from outside the droplets into the droplets for separation, storage and/or reaction of compounds inside the droplets, wherein the droplets comprise a polymer according to Formula (I)

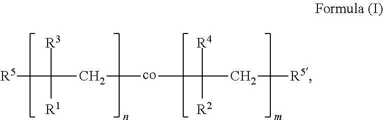

wherein
n is an integer selected from 0 to 2500, optionally 1 or 2 to 2500, optionally from 25 to 250;
m is an integer selected from 2 to 2500, optionally from 25 to 2500, from 25 to 250 or from 50 to 250;
R³ and R⁴ are each independently selected from the group consisting of hydrogen and methyl;
R⁵ and R^{5′} are independently selected from
   hydrogen, linear or branched (C₁₋₁₀)alkyl, a halide and phenyl;
   a group resulting from a polymerization initiator, optionally a group resulting from a RAFT polymerization initiator, optionally

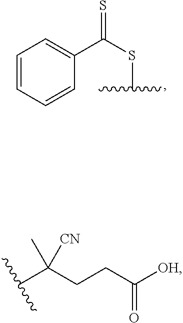

or
   a target binding moiety (TBM), optionally a TBM attached to or reacted with a group resulting from a polymerization initiator, optionally from a RAFT polymerization initiator, optionally

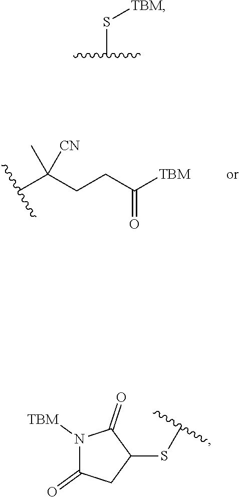

wherein the TBM is optionally selected from the group consisting of azide, alkyne, carboxylate, amine, thiol, hydroxyl, aldehyde, cyanate, sulfonyl, tosyl, tresyl, epoxide, carbonate, anhydride, carbamate, imidazole, azlactone, triazine, maleimide, aziridine, peroxide, acyl, anthraquinone, diazo, diazirine, psoralen, NHS ester, imido ester, (C₂₋₁₀)alkenyl or alkyl groups, DBCO, cytosine, guanidine, streptavidin, avidin, biotin, an aptamer, a peptide, optionally a peptide comprising an affinity tag, optionally an affinity tag selected from FLAG, HIS and ALFA, a protein, an oligonucleotide, a positively or negatively charged oligopolymer, a nucleic acid, a carbohydrate, a dye, a cell, an antibody, an organic and inorganic nanoparticle, and chelating agents, optionally ethylenediaminetetraacetic acid (EDTA),
   optionally wherein at least one of R⁵ and R^{5′} is a TBM if n=0;
R¹ is selected from the group consisting of

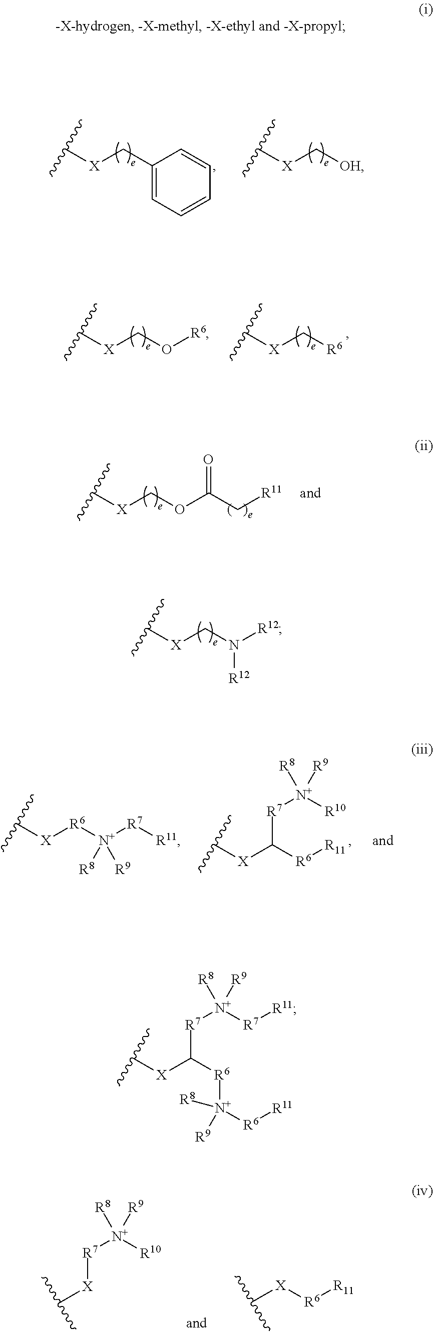

in a ratio of about 1:1 within the polymer;

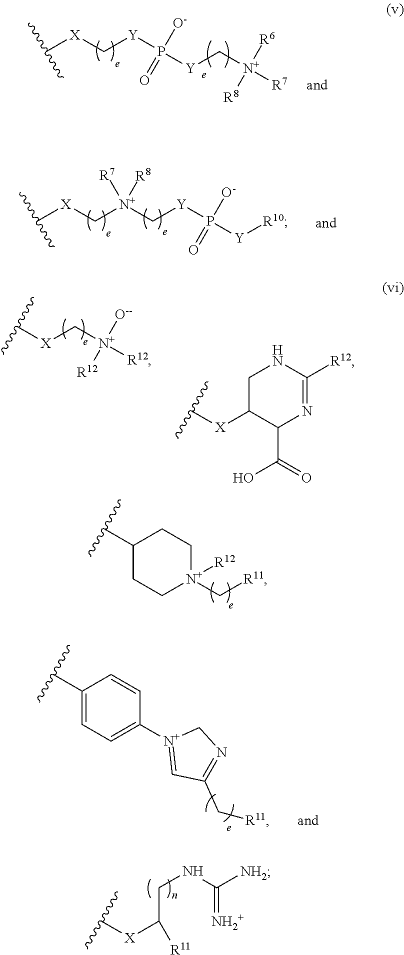

R² is selected from the group consisting of

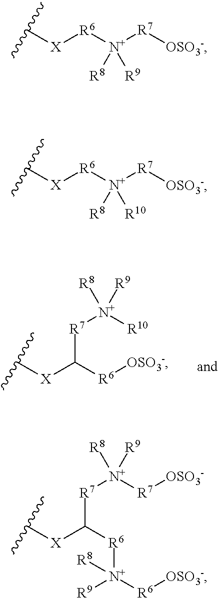

wherein
X is selected from the group consisting of —C(═O)—O—, —C(═O)—NH—, aliphatic and aromatic carbo- or heterocycle;
Y is oxygen or absent;
e is an integer selected from 1 to 5, optionally from 2 to 3;
R⁶ and R⁷ are each independently selected from the group consisting of
   linear or branched, substituted or non-substituted (C₁₋₁₀)alkyl, optionally (C₁₋₅)alkyl, optionally methyl, ethyl and propyl,
   linear or branched, substituted or non-substituted (C₁₋₁₀)alkyl, optionally (C₂₋₆)alkyl chains, comprising one or more heteroatoms selected from the group consisting of O, N, S and P, optionally (C₂₋₅)alkyl ethers,
   (C₂₋₁₀)alkenyl, phenyl, and an aromatic heterocycle, optionally pyridine, pyrrole, furan and imidazole;
R⁸, R⁹ and R¹⁰ are each independently selected from the group consisting of hydrogen, linear or branched, substituted or non-substituted (C₁₋₁₀)alkyl, optionally (C₁₋₅)alkyl, optionally methyl, ethyl and propyl, (C₂₋₁₀)alkenyl, phenyl, an aromatic heterocycle, optionally pyridine, pyrrole, furan and imidazole,

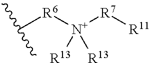

when forming R¹,

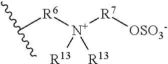

when forming R²;
R⁸ and R⁹ together or two R¹³ together optionally form an aliphatic or aromatic heterocycle, optionally a piperidine ring, a piperazine ring or a morpholine ring;
R⁸, R⁹ and R⁶ or R⁸, R⁹ and R⁷ optionally together form a five or six-membered ring, optionally a pyridine ring, a piperidine ring, pyrrole ring, a pyrimidine ring, a pyrazole ring, an imidazole ring, a pyrazine ring, an isoxazole ring or an oxazole ring; wherein one of R⁸ or R⁹ is absent if R⁸ and R⁶, R⁹ and R⁶ or R⁸ and R⁷, or R⁹ and R⁷ together form an aromatic ring;
R¹¹ is selected from the group consisting of —SO₃⁻, —C(═O)O⁻, and

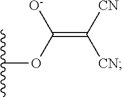 [SMILES: *OC([O-])=C(C#N)C#N]

R¹² is selected from the group consisting of methyl, ethyl and propyl;
R¹³ is independently selected from the group consisting of linear or branched, substituted or
non-substituted (C₁₋₁₀)alkyl, optionally (C₁₋₅)alkyl, optionally methyl, ethyl and propyl, (C₂₋₁₀)alkenyl, phenyl, an aromatic heterocycle, optionally pyridine, pyrrole, furan and imidazole,
wherein the polymer optionally is crosslinked by a crosslinker, optionally a crosslinker as R¹.

In an embodiment, R⁵ and R^{5′} are independently selected from a group resulting from a polymerization initiator, optionally a group resulting from a RAFT polymerization initiator, optionally,

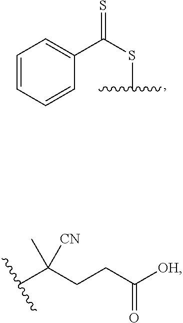

or
a target binding moiety (TBM), optionally a TBM attached to or reacted with a group resulting from a polymerization initiator, optionally from a RAFT polymerization initiator, optionally

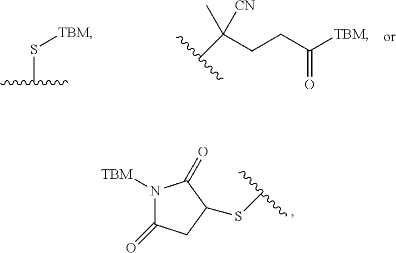

wherein the TBM is optionally selected from the group consisting of azide, alkyne, carboxylate, amine, thiol, hydroxyl, aldehyde, cyanate, sulfonyl, tosyl, tresyl, epoxide, carbonate, anhydride, carbamate, imidazole, azlactone, triazine, maleimide, aziridine, peroxide, acyl, anthraquinone, diazo, diazirine, psoralen, NHS ester, imido ester, (C₂₋₁₀)alkenyl or alkyl groups, DBCO, cytosine, guanidine, streptavidin, avidin, biotin, an aptamer, a peptide, optionally a peptide comprising an affinity tag, optionally an affinity tag selected from FLAG, HIS and ALFA, a protein, an oligonucleotide, a positively or negatively charged oligopolymer, a molecularly imprinted polymer, an affinity polymer, a nucleic acid, a carbohydrate, a dye, a cell, an antibody, an organic and inorganic nanoparticle, and chelating agents, optionally ethylenediaminetetraacetic acid (EDTA); optionally wherein at least one of R⁵ and R^{5′} is a TBM if n=0.

In an embodiment, at least one of R⁵ and R^{5′} is a TBM if
R¹ is selected from

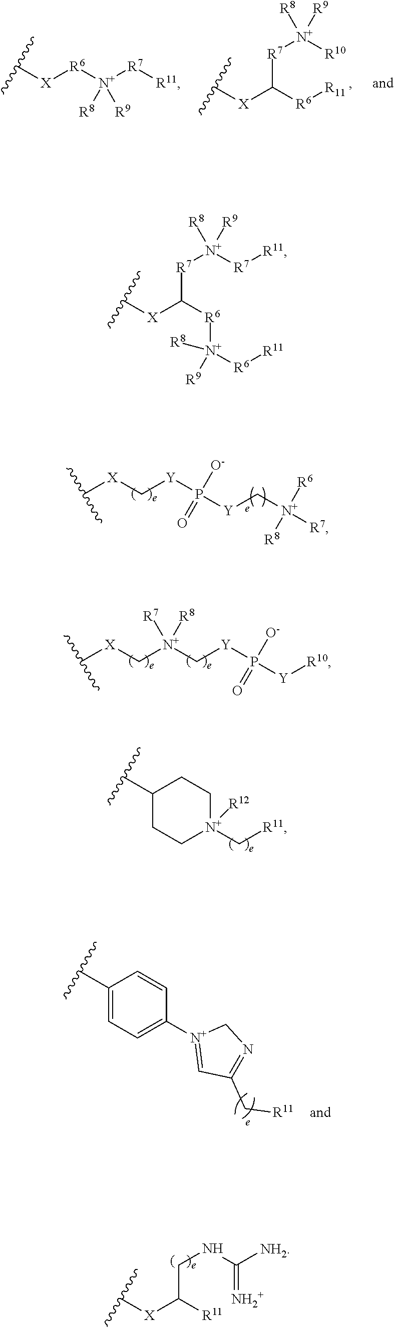

In this embodiment, it is evident that n is not zero if R¹ is present.

All definitions, examples and explanations provided herein are valid for all aspects and embodiments of the present invention if not specifically indicated otherwise.

The droplets comprising the polymer for use as described herein for all aspects and embodiments are liquid in an aqueous medium, which means that they do not form solids but are coacervates with an essentially spherical shape, e.g. of about 3 to 100 μm in diameter, that are able to coalesce, e.g. to form larger droplets or a continuous phase. Moreover, and for example, the droplets exhibit recovery in order of seconds in fluorescence recovery after photobleaching (FRAP) experiments.

In the context of the present invention, the term “comprising a polymer according to formula (I)” means comprising at least one, i.e. one or more (different) polymers according to formula (I).

The polymers of the droplets described herein for all aspects and embodiments are able to encode electrostatic attractive interactions, leading to the formation of the liquid droplets with many relevant features, for example: i) the droplets present low interfacial tension and, for this reason, generally do not require a high amount of energy to be generated; ii) the droplets can reversibly assemble and disassemble upon changes in the surrounding environment (e.g. by changing either temperature or ionic strength); and iii) the droplets can avoid unspecific protein adsorption on their surface and preferentially exclude many solutes. All these features are important when using these droplets for isolation of specific components in a mixture. If more than one polymer is present in the liquid droplets, e.g. two different polymers according to formula (I) or one polymer according to formula (I) and another polymer according to a different formula, the different polymers may have different functions, e.g. one polymer may induce phase separation and the other polymer may change the surface properties of the liquid droplet. When functionalized with a targeting agent (e.g. antibodies, biotin, etc. which can be easily conjugated to the polymer as R⁵ and/or R^{5′}), the droplets can selectively interact with the biomolecule of interest and preferentially exclude all other components. The droplets can be therefore used to, e.g., upconcentrate and separate only the desired compound from complex mixtures. Also, the targeting agent can aid in an upconcentration which goes beyond the statistical distribution of targeted compounds. The liquid nature of the droplet and the spherical shape (i.e. high surface to volume ratio) are important aspects to increase the solute upconcentratrion due to the presence of a dynamic interface. Moreover, the possibility to choose different targeting agents confers modularity and flexibility. Additionally, the technology involving the droplets described herein is also easily scalable.

Thanks to these properties, these smart synthetic droplets can be used in many applications, in particular for diagnostics and for the purification of biopharmaceuticals. In the case of diagnostics, these droplets are able to, e.g., preferentially recruit pathological biomarkers from complex fluids and to detect them without the need of further purification steps, in sharp contrast with the methods developed so far (e.g. magnetic beads or ELISA). In fact, the droplets act as signal concentrator. In addition, the present invention can be easily implemented in microfluidics devices allowing the use of small amounts of samples and the increase of the solute concentration factor, e.g. at least up to 10⁵. This is an aspect not achievable at bulk scale with state of the art devices. The droplets can also be used as pre-isolation tool for further processing in the context of biomarker detection for disease diagnosis.

Based on pre-designed polymer solubility properties, the change in solubility of the polymer for use as droplets described herein, upon target binding can also be used to detect the presence of target compounds which results in the shrinkage of the droplets and allows the measurement of target compounds without requiring any secondary labeling step. A first target application can be the field of disease diagnosis. The droplets of the present invention could also be used to measure binding affinities, which would be of high relevance for instance for drug discovery, where little amount of candidate biotherapeutics are available, and are usually not purified and therefore need to be characterized directly in their complex environment.

The droplets could further be used as tissue engineering scaffolds. A possible application would be scaffolds for cell culture growth. Once cell clusters reach a defined size, issues may arise inside the cluster to transport essential metabolites to the most inner cells at the core of the cluster, to remove toxic metabolites and transport oxygen. If constant environment is not guaranteed, this can lead to cell death, or cell differentiation in the case of stem cell cultures. Here, the unique properties of the liquid droplets described herein can be used to induce spontaneous or activated splitting of the cell cluster once it has reached a critical size in order to ensure sufficient mass transport and a constant microenvironment during cell culture growth.

In the case of purification of biopharmaceuticals, the liquid droplets can be used to selectively separate one or more products (i.e. desired products or contaminants) from complex media. The droplets allow, e.g., the recruited products to be separated from the rest of the media, e.g. through centrifugation or filtration, and then be released in a different buffer. The release can be achieved, e.g., by applying an external stimulus that interferes with the interaction between the polymer and the products, e.g. a change in the ionic strength or pH, the addition of competitive agents or a combination of these. The droplets can be engineered for affinity, ion exchange and hydrophobic interaction purifications. For example, the polymer composition can be adjusted to control the polymer size and to ensure that the uptake and release of the products are performed in conditions that do not damage the product. Moreover, the size and the surface properties (e.g. surface tension and wetting) of the droplets can be tuned, e.g., by mixing different polymers. The design space of the polymers can be further expanded when the polymers are crosslinked. Unlike chromatography, the liquidity of the droplets allows the system to dynamically adapt to the amount of product in solution, that often is not constant as a result of productivity fluctuations in upstream processes. Unlike precipitation, the droplets prevent product aggregation allowing an easier product recovery and making this technology a gentle method for the purification of biopharmaceuticals. This technology is scalable and can be used for purifications in batch and continuous modes.

For drug discovery applications, the liquid droplets can be functionalized with the desired target, and binding libraries of e.g. antibodies or aptamers or other binding agents can be tested. The binding affinity between the target and the binding agent can be measured by monitoring the change in droplet properties upon binding, such as signal increase in the droplet due to the selective uptake of the binding agent, or changes in solubility properties upon binding measured by the Tcp, or the decrease in droplet size. A key advantage is that the preferential exclusion of the droplet material allows to perform these operations in complex mixtures, such as cell lysates, or partially purified binding agent libraries.

The notation of

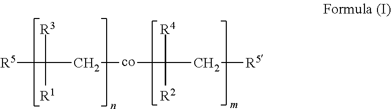

and any other formula herein which discloses a polymer by means of n- and m-numbers of building blocks, is to be understood as follows. The “co”, as defined in IUPAC nomenclature, means that the sequence of monomers within the polymer is not specified, i.e. there is an unspecified sequence of monomers in the polymer. Optionally, the distribution of the monomers can be alternating, statistical, or in the form of a block co-polymer. R⁵ and R^{5′} on either end (alpha and omega end) of the polymer can be a chemical entity or group resulting from the reaction of a polymerization initiator, which may be further functionalized by a TBM. Exemplary chemical entities resulting from polymerization reactions include, e.g., groups resulting from a reversible addition-fragmentation chain transfer (RAFT) reagent, an atom transfer radical polymerization (ATRP) reagent or free-radical polymerization (FRP) reagent. These reagents and the products of the reagents that are formed as groups on the polymer ends are well known in the art. For example, RAFT reagents are commercially available trithiocarbonates, dithiocarbamates, dithiobenzoates, switchable RAFT reagents or macro-RAFT reagents, e.g. as available from Sigma Aldrich Canada Co. or Merck KGaA, Darmstadt, Germany. It is within the skilled person's general chemical knowledge what the structure of R⁵ and/or R^{5′} will be when choosing a specific polymerization initiator. Alternatively, R⁵ and/or R^{5′} may be a chemical entity resulting from a polymerization reaction as described herein which entity is further chemically modified (e.g. reacted with a TBM) to be a target binding moiety, or R⁵ and/or R^{5′} may be a target binding moiety in the absence of a chemical entity resulting from a polymerization. The term “a TBM attached to or reacted with a group resulting from a polymerization initiator” means that a TBM is attached to the group by means of a chemical reaction to form, e.g. a covalent or ionic, bond with the chemical entity resulting from a polymerization. This attachment may result in the direct attachment of the TBM to the polymer under loss of the chemical entity resulting from a polymerization or it may result in the chemical modification of the chemical entity, e.g. partial loss of chemical entity.

In any case, the target binding moiety is optionally selected from the group consisting of azide, alkyne, carboxylate, amine, thiol, hydroxyl, aldehyde, cyanate, sulfonyl, tosyl, tresyl, epoxide, carbonate, anhydride, carbamate, imidazole, azlactone, triazine, maleimide, aziridine, peroxide, acyl, anthraquinone, diazo, diazirine, psoralen, NHS ester, imido ester, (C₂₋₁₀)alkenyl or alkyl groups, DBCO, cytosine, guanidine, streptavidin, avidin, biotin, an aptamer, a peptide, a protein, an oligonucleotide, a nucleic acid, a carbohydrate, a dye, a cell, an antibody, an organic and inorganic nanoparticle, positively and negatively charged oligopolymers, a molecularly imprinted polymer, and an affinity polymer.

The polymer described herein for all aspects and embodiments may be crosslinked by any suitable crosslinker. A crosslinker is a chemical agent that crosslinks the polymer, i.e. binds together two or more polymer chains, by attaching via at least two moieties of the same crosslinker to two or more polymer chains. The crosslinking can be reversible or irreversible and it can be based on a chemical linkage or on non-covalent interactions between the crosslinker and the polymer. Suitable crosslinkers for polymers are known to the skilled person and include but are not limited to, e.g. bismethacrylates, bismethacrylamides, and glutharaldeyde. Examples of non-covalent crosslinkers include, e.g., streptavidin that is able to bind with biotin-conjugated polymers or nanoparticles/proteins/compounds decorated with multiple antibodies or targeting agents.

The target binding moiety (TBM), as used herein for all aspects and embodiments, includes any suitable chemical or biological moiety that can bind a desired target, e.g. a compound to be taken up by the droplets, reversibly or irreversibly, covalently or non-covalently. Optionally, the target binding moiety is chosen such that it binds only to the desired product when in use together as part of the liquid droplets described herein. Examples for reactive groups or moieties for bioconjugations are known in the art (see, e.g., Greg T. Hermanson, Chapter 3—The Reactions of Bioconjugation, Bioconjugate Techniques (Third Edition), 2013, Pages 229-258). For example, the TBM can be

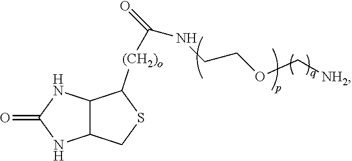

wherein o in (CH₂)o is an integer from 1 to 10, optionally 2 to 5, p is an integer from 1 to 5, optionally 2, and q is an integer from 1 to 5, and the TBM is connected via the amine to the polymer directly or to the entity resulting from polymerization described herein. Optionally, the TBM can be

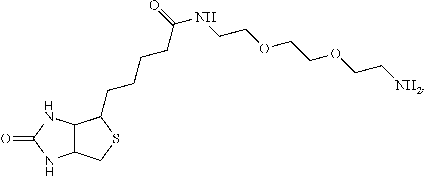 [SMILES: NCCOCCOCCNC(=O)CCCCC1SCC2NC(=O)NC21]

attached via the amine to the polymer described herein, optionally to R⁵ or R^{5′} being

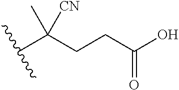 [SMILES: *C(C)(C#N)CCC(=O)O]

and from an amide bond.

The term DBCO, as used herein, refers to dibenzocyclooctyne, which is useful, e.g., in strain-promoted copper-free azide-alkyne cycloaddition reactions.

An antibody, as used herein for all aspects and embodiments, includes functional fragments and functional derivatives thereof or antibody-like binding proteins that bind a desired target, e.g. a compound to be taken up by the droplets. As used herein, the term antibody is meant to include whole antibodies, functional fragments and functional derivatives thereof that specifically bind a desired target. These are routinely available by hybridoma technology (Kohler and Milstein, Nature 256, 495-497, 1975), antibody phage display (Winter et al., (1994) Annu. Rev. Immunol. 12, 433-455), ribosome display (Schaffitzel et al., (1999) J. Immunol. Methods, 231, 119-135) and iterative colony filter screening (Giovannoni et al., (2001) Nucleic Acids Res. 29, E27) once the desired target is available. Typical proteases for fragmenting antibodies into functional products are well-known. Other fragmentation techniques can be used as well as long as the resulting fragment has a specific high affinity and, preferably a dissociation constant in the micromolar to picomolar range. A very convenient antibody fragment for targeting applications is the single-chain Fv fragment, in which a variable heavy and a variable light domain are joined together by a polypeptide linker. Other antibody fragments for target binding include Fab fragments, Fab₂ fragments, miniantibodies (also called small immune proteins), tandem scFv-scFv fusions as well as scFv fusions with suitable domains (e.g. with the Fc portion of an immuneglobulin). For a review on certain antibody formats, see Holliger P, Hudson P J.; Engineered antibody fragments and the rise of single domains. Nat Biotechnol. 2005 September, 23(9):1126-36). The term “functional derivative” of an antibody for use in the present invention is meant to include any antibody or fragment thereof that has been chemically or genetically modified in its amino acid sequence, e.g. by addition, substitution and/or deletion of amino acid residue(s) and/or has been chemically modified in at least one of its atoms and/or functional chemical groups, e.g. by additions, deletions, rearrangement, oxidation, reduction, etc. as long as the derivative has substantially the same binding affinity as to its original antigen and, optionally, has a dissociation constant in the micro-, nano- or picomolar range. The antibody, fragment or functional derivative thereof for use in the present invention can be, for example, one that is selected from the group consisting of polyclonal antibodies, monoclonal antibodies, chimeric antibodies, humanized antibodies, CDR-grafted antibodies, Fv-fragments, Fab-fragments and Fab₂-fragments and antibody-like binding proteins, e.g. affilines, anticalines and aptamers, any protein-ligand binding pair and the fragments of the both. For a review of antibody-like binding proteins see Binz et al. on engineering binding proteins from non-immunoglobulin domains in Nature Biotechnology, Vol. 23, No. 10, October 2005, 12571268. The term “aptamer” describes nucleic acids that bind to a polypeptide with high affinity. Aptamers can be isolated from a large pool of different single-stranded RNA molecules by selection methods such as SELEX (see, e.g., Jayasena, Clin. Chem., 45, p. 1628-1650, (1999); Klug and Famulok, M. Mol. Biol. Rep., 20, p. 97-107 (1994); U.S. Pat. No. 5,582,981). Aptamers can also be synthesized and selected in their mirror form, for example, as the L-ribonucleotide (Nolte et al., (1996) Nat. Biotechnol., 14, pp. 1116-1119; Klussmann et al., (1996) Nat. Biotechnol. 14, p. 1112-1115). Forms isolated in this way have the advantage that they are not degraded by naturally occurring ribonucleases and, therefore, have a greater stability. Another antibody-like binding protein and alternative to classical antibodies are the so-called “protein scaffolds”, for example, anticalines, that are based on lipocaline (Beste et al., (1999) Proc. Natl. Acad. Sci. USA, 96, p. 1898-1903). The natural ligand binding sites of lipocalines, for example, of the retinol-binding protein or bilin-binding protein, can be changed, for example, by employing a “combinatorial protein design” approach, and in such a way that they bind selected haptens (Skerra, (2000) Biochem. Biophys. Acta, 1482, pp. 337-350). For other protein scaffolds it is also known that they are alternatives for antibodies (Skerra, (2000) J. Mol. Recognit, 13, pp. 167-287; Hey, (2005) Trends in Biotechnology, 23, pp. 514-522). In summary, the term functional antibody derivative is meant to include the above protein-derived alternatives for antibodies, i.e. antibody-like binding proteins, e.g. affilines, anticalines and aptamers that specifically recognize a desired target, compound, polypeptide, fragment or derivative thereof.

Examples for dyes, as used herein for all aspects and embodiments include, e.g., ATTO-488, ATTO390, ATTO647N, ATTO565, ALEXA488, Rhodamine B, Fluorescein, Amplex Red, fluorescamine, aromatic dialdehyde naphthalene-2,3-dicarboxaldehyde (NDA), or fluoraldehyde.

Examples for cells, as used herein for all aspects and embodiments include, e.g., stem cells, dendritic cells, microglia, beta cells, T cells, lymphocytes, astrocytes, E. coli, osteoblasts, or HEK cells.

Examples for organic and inorganic nanoparticles, as used herein for all aspects and embodiments include, e.g., magnetic nanoparticles, quantum dots, metal-organic framework (MOF) nanoparticles.

Examples for positively and negatively charged oligopolymers, as used herein for all aspects and embodiments, include, e.g., poly(methacrylic acid sodium salt), poly(2-dimethylamino)ethyl methacrylate) methyl chloride quaternary salt, and sodium alginate.

Examples for carbohydrates include chitosan and heparin.

As used herein, a molecularly imprinted polymer is a polymer that can selectively bind the product with which it was imprinted during production. Examples for molecularly imprinted polymers are imprinted acrylic and methacrylic polymers (BelBruno, (2019), Chem. Rev., 119, 1, pp. 94-119).

Affinity polymers include, e.g., epitope-selective linear copolymers (Latza, (2014), Chem. Eur. J., 20, pp. 11479-11487).

The controlled uptake of compounds from outside the droplets into the droplets for storage, separation and/or reaction of compounds inside the droplets means that the droplets selectively take up compounds from the environment, i.e. exterior of the droplets, into the droplets and store these compounds inside the droplets without significant leakage of the same compounds. Once stored, the compounds may undergo chemical or biological reactions within the droplets and subsequently may or may not be released from the droplets, e.g. based on their altered chemical composition or properties. The separation of the compounds inside the droplets can refer to the separation of compounds originating from outside the droplets and it can refer to the separation of different compounds located within the droplets, e.g. based on different chemical properties of the compounds that interact with different TBMs or R¹/R² structures of the polymer of the droplets, e.g. leading to compartments or sub-compartments within the same droplet for compound separation as described further below.

The term “controlled” as used herein in the context of the uptake means that the uptake can be controlled, e.g. the relative amount of compounds inside and outside the droplets can be controlled. Means of controlling the uptake (or the relative amount of compounds inside and outside the droplets) include adjusting the environment of the liquid droplets, e.g. by an external stimulus, e.g. by adjusting the pH of the aqueous medium, the temperature, or the ionic strength of the aqueous medium. Also, the addition of further compounds to the environment of the liquid droplets (the aqueous medium) such as salts, proteins, antibodies or vesicles can change the environment and lead to an external stimulus that controls the uptake. The controlled uptake does not necessarily correlate with the formation or dissolution of the liquid droplets. For example, a compound can be taken up by the liquid droplets at a given ionic strength of the aqueous medium and be released at a different, e.g. higher ionic strength without dissolving the liquid droplets.

Optionally, the term “controlled” means that the uptake is selective for a given type, class or specific compound.

The process described herein for uptake of compounds from outside the droplets into the droplets is not to be confused with the loading of, e.g. solid, vesicles in the context of drug delivery. The droplets for use in the present invention are not used or assembled together with high concentrations of a compound in order to load the compound into the droplets. Rather, the droplets are used in environments where the compounds should be taken up by the already existing droplets, e.g. at relatively low concentrations of the compounds, into the droplets and subsequently are not released from the droplets without alteration of the compounds or the droplets.

The term “compound” refers to any chemical and biological species that can be taken up by the droplets. These include small molecules, i.e. organic molecules with a molecular weight in the range of 10-10000 Da, which can be polar or apolar, aliphatic or aromatic, hydrophilic, amphiphilic or lipophilic; proteins with a molecular weight in the range of 5-2000 kDa, e.g. antibody-based drugs, Fc fusion proteins, anticoagulants, blood factors, enzymes, growth factors, hormones, interferons, interleukins, thrombolytics, engineered protein scaffolds, ab42; protein complexes; peptides with a molecular weight in the range of 100-10000 Da; nucleic acids including antisense oligonucleotides, RNA, messenger RNA, circular RNA, RNA interference drugs, ribozymes, aptamers, DNA, plasmid DNA, DNAzymes; particles with diameters in the range of 25 to 2000 nm, e.g. lipoproteins, exomeres, exosomes, microvesicles, apoptotic bodies, lentiviruses, retroviruses, adenoviruses, adeno-associated viruses, viral-like particles, inactivated viruses, liposomes, lipid particles, polymerosomes; carbohydrates including monosaccharides, oligosaccharides and polysaccharides; cells including bacterial, yeast and mammalian cells; contaminants, e.g. mercury, heavy metals, fluropolymers (e.g. perfluorooctanoic acid (PFOA), perfluorooctanesulfonic acid (PFOS)), polycyclic aromatic hydrocarbons (PAHs), dioxins bipyridylium, carbamates, organochlorines, pyrethroids, triazines, organophosphates, microplastics, nanoplastics, persistent organic pollutants (POPs), environmental persistent pharmaceutical pollutants (EPPPs) (e.g. estradiol, ethinylestradiol, diclofenac, antibiotics).

As used herein for all aspects and embodiments, a “substituent” or “residue” or “R”, refers to a molecular moiety that is covalently bound to an atom within a molecule of interest. For example, a “substituent”, “R” or “residue” may be a moiety such as a halogen, alkyl group, haloalkyl group or any other substituent described herein that is covalently bonded to an atom, optionally a carbon, oxygen or nitrogen atom, that forms part of a molecule of interest.

The group X used herein for all aspects and embodiments is selected from the group consisting of —C(═O)—O—, —C(═O)—NH—, aliphatic and aromatic carbo- or heterocycle. In any aspect disclosed herein, the group “—C(═O)—O—” or “—C(═O)—NH—” is annotated such that the attachment point (indicated by the waved line) of the moiety comprising said group is at the beginning of either —C(═O)—O— or —C(═O)—NH—. In other words and as a representative example for all aspects and embodiments, if X is selected to be —C(═O)—O— or —C(═O)—NH—in a residue of the following formula:

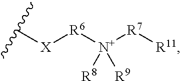

the following structures result:

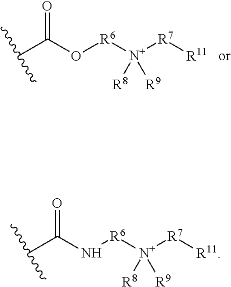

The term “non-substituted” as used herein shall mean substituted only with hydrogen. The term “substituted” as used herein, means that any one or more hydrogens on the designated atom or group is replaced, independently, with an atom different from hydrogen, optionally by a halogen, optionally by fluorine, a thiol, a carboxyl, a cyano, a nitro, an alkyl (optionally C₁-C₁₀), aryl (optionally phenyl, benzyl or benzoyl), an alkoxy group, a sulfonyl group, by a tertiary or quaternary amine or by a selection from the indicated substituents, provided that the designated atom's normal valence is not exceeded, and that the substitution results in a stable compound, i.e., a compound that can be isolated and characterized using conventional means.

The term “heteroatom” as used herein shall be understood to mean atoms other than carbon and hydrogen such as, e.g., O, N, S and P.

In the context of the present invention it is understood that antecedent terms such as “linear or branched”, “substituted or non-substituted” indicate that each one of the subsequent terms is to be interpreted as being modified by said antecedent term. For example, the scope of the term “linear or branched, substituted or non-substituted alkyl, alkenyl, alkynyl, carbocycle” encompasses linear or branched, substituted or non-substituted alkyl; linear or branched, substituted or non-substituted alkenyl; linear or branched, substituted or non-substituted alkynyl; linear or branched, substituted or non-substituted alkylidene; and linear or branched, substituted or non-substituted carbocycle. For example, the term “(C₂₋₁₀) alkenyl, alkynyl or alkylidene” indicates the group of compounds having 2 to 10 carbons and alkenyl, alkynyl or alkylidene functionality.

The expression “alkyl” refers to a saturated, straight-chain or branched hydrocarbon group that contains the number of carbon items indicated, e.g. linear or branched “(C₁₋₁₀)alkyl” denotes a hydrocarbon residue containing from 1 to 10 carbon atoms, e.g. a methyl, ethyl, propyl, iso-propyl, n-butyl, iso-butyl, sec-butyl, tert-butyl, n-pentyl, iso-pentyl, n-hexyl, 2,2-dimethylbutyl, etc.

The expression “alkenyl” refers to an at least partially unsaturated, substituted or non-substituted straight-chain or branched hydrocarbon group that contains the number of carbon atoms indicated, e.g. “(C₂₋₁₀)alkenyl” denotes a hydrocarbon residue containing from 2 to 10 carbon atoms, for example an ethenyl (vinyl), propenyl (allyl), iso-propenyl, butenyl, isoprenyl or hex-2-enyl group, or, for example, a hydrocarbon group comprising a methylene chain interrupted by one double bond as, for example, found in monounsaturated fatty acids or a hydrocarbon group comprising methylene-interrupted polyenes, e.g. hydrocarbon groups comprising two or more of the following structural unit —[CH═CH—CH₂]—, as, for example, found in polyunsaturated fatty acids. Alkenyl groups have one or more, e.g. 1, 2, 3, 4, 5, or 6 double bond(s).

The expression “alkyne” refers to at least partially unsaturated, substituted or non-substituted straight-chain or branched hydrocarbon groups that may contain, e.g. from 2 to 10 carbon atoms, for example an ethinyl, propinyl, butinyl, acetylenyl, or propargyl group. Optionally, alkine groups have one or two (e.g. one) triple bond(s).

Furthermore, the terms “alkyl”, “alkenyl” and “alkyne” also refer to groups in which one or more hydrogen atom(s) have been replaced, e.g. by a halogen atom, optionally F, Cl or Br, such as, for example, a 2,2,2-trichloroethyl, tribromoethyl or a trifluoromethyl group.

The term “heterocycle” refers to a stable substituted or non-substituted, aromatic or non-aromatic, optionally 3 to 10 membered, optionally 3-6 membered, optionally 5 or 6 membered, monocyclic, heteroatom-containing cycle. Each heterocycle consists of carbon atoms and one or more, optionally 1 to 4, optionally 1 to 3 heteroatoms optionally chosen from nitrogen, oxygen and sulphur. A heterocycle may contain the number of carbon atoms in addition to the non-carbon atoms as indicated: a “(C₃₋₆)heterocycle” is meant to have 3 to 6 carbon atoms in addition to a given number of heteroatoms.

Exemplary heterocycles include, but are not limited to pyridine, piperidine, pyrrole, pyrimidine, pyrazole, imidazole, imidazole, pyrazine, isoxazole or oxazole.

The expressions “alkyl ether” refers to a saturated or non-saturated, straight-chain or branched hydrocarbon group that contains the number of carbon items indicated. For example, “(C₂₋₅)alkyl ether” denotes a hydrocarbon residue containing from 2 to 5 carbon atoms, and any suitable number of oxygen atoms that will result in an ether structure. Alkyl ether groups as used herein, shall be understood to mean any linear or branched, substituted or non-substituted alkyl chain comprising an oxygen atom either as an ether motif, i.e. an oxygen bound by two carbons. Exemplary alkyl ethers are polyethylene glycol (PEG) chains. The term polyethylene glycol as used herein refers to a chain of substituted or non-substituted ethylene oxide monomers. The ether residue can be attached to the Formula provided in the present invention either via the carbon atom or via the oxygen atom of the ether residue.

If more than one residue R is attached to a given atom of a formula described herein, one residue R can be absent if the attachment of the other R leads to a full valency of the atom. For example, R⁸ or R⁹ is absent if R⁸ and R⁶, R⁹ and R⁶ or R⁸ and R⁷, or R⁹ and R⁷ together form an aromatic ring.

As used herein, a wording defining the limits of a range of length such as, e. g., “from 1 to 5” or “(C₁₋₅)” means any integer from 1 to 5, i.e. 1, 2, 3, 4 and 5. In other words, any range defined by two integers explicitly mentioned is meant to comprise and disclose any integer defining said limits and any integer comprised in said range.

The scope of the present invention includes those analogs of the compounds as described above and in the claims that feature the exchange of one or more carbon-bonded hydrogens, optionally one or more aromatic carbon-bonded hydrogens, with halogen atoms such as F, Cl, or Br, optionally F. The exchange of one or more of the carbon-bonded hydrogens, e.g. by fluorine, can be done, e.g., for reasons of metabolic stability and/or pharmacokinetic and physicochemical properties.

In an embodiment, the droplets as such or in the context of the use of the present invention are multiphase immiscible droplets, wherein compartments of at least two different droplets can be fully immiscible or form sub-compartments. The compartments are to be understood as the “interior” of the droplets. The sub-compartments of at least two different droplets may have a core-shell morphology and/or feature partial wetting, e.g. as shown in FIGS. 5A-5D. The full or partial immiscibility of the at least two droplets described herein for any aspect is based on the different chemical nature of the polymer of the at least two droplets.

For example, when two different polymers described herein are mixed, these two form distinct immiscible droplets that are composed only of one single type of polymer (i.e. if polymer A is mixed with polymer B, two types of droplets are obtained, wherein one type of droplet is composed only of polymer A and the other one is composed only of polymer B). These immiscible droplets can be fully separated, partially wetting or feature a core shell morphology.

As an example, the above properties of the droplets to separate based on the nature of the polymer they are made of can be used to detect two or more different compounds (e.g. biomarkers) at the same time from the same complex mixture. At the end, multiple immiscible droplets each containing a specific compound or biomarker can be obtained and detected simultaneously from the same complex sample mixture (e.g. a blood sample).

For example, fine-tuning the different responsiveness of the droplets may be used to recover two recruited compounds. As an example, the droplets can be sequentially disassembled and the compounds can be recovered at different pH, temperature or ionic strength.

In an embodiment of all aspects descried herein, R⁵ and/or R^{5′} are selected from

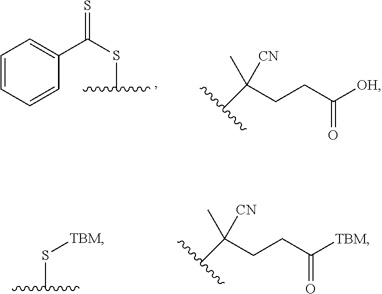

optionally

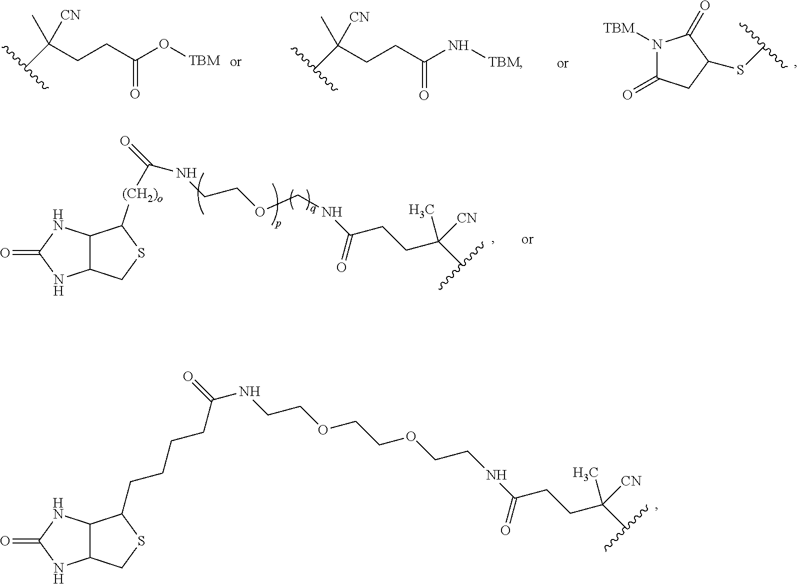

wherein o in (CH₂)o is an integer from 1 to 10, optionally 2 to 5, p is an integer from 1 to 5, optionally 2, and q is an integer from 1 to 5.

In an embodiment, the use of the present invention is one, wherein
n is an integer selected from 10 to 250;
m is an integer selected from 10 to 250;
e is an integer selected from 2 to 3;
X is selected from the group consisting of —C(═O)—O—, —C(═O)—NH—, pyridine, piperidine and phenyl;
R⁶ and R⁷ are each independently selected from the group consisting of (C₁₋₅)alkyl, optionally methyl, ethyl and propyl;
R⁸, R⁹ and R¹⁰ are each independently selected from the group consisting of methyl,

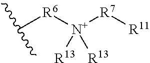

when forming R¹, and

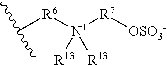

when forming R²;
R¹¹ is —SO₃⁻ or —C(═O)O⁻;
R¹² is methyl; and
R¹³ is selected from the group consisting of methyl, ethyl and propyl.

In an embodiment, the use of the present invention is one, wherein
R¹ is selected from the group consisting of

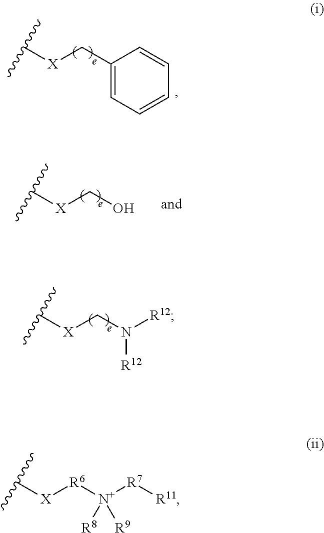

optionally

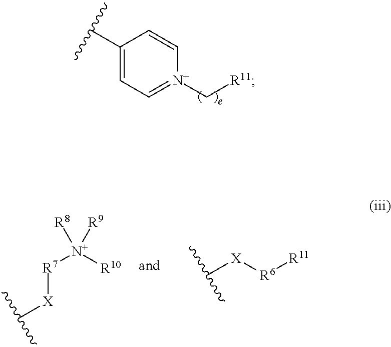

in a ratio of about 1:1 within the polymer;

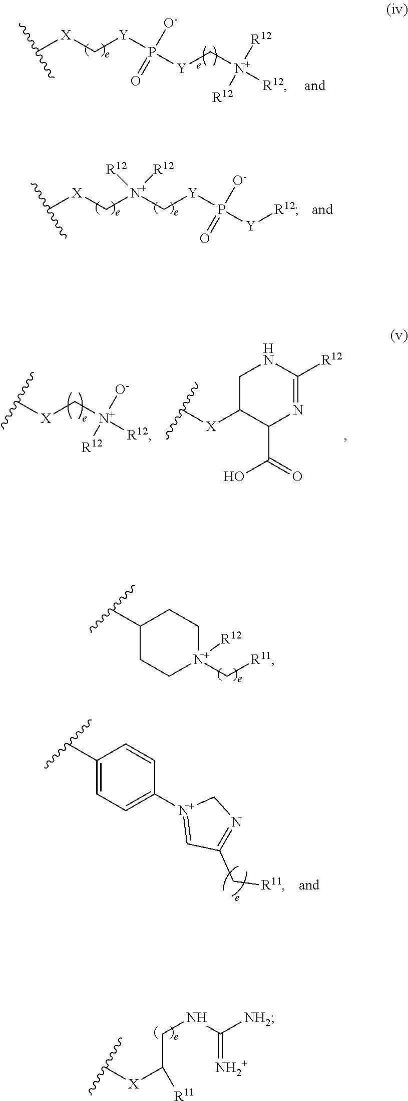

and
R² is

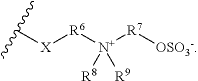

In an embodiment, the use of the present invention is one, wherein n is an integer selected from 10 to 250;
m is an integer selected from 10 to 250;
R⁵ and R^{5′} are hydrogen or independently selected from the group consisting of azide, carboxylic acid, amine, (C₂₋₁₀)alkenyl, streptavidin, avidin, biotin, an aptamer, a peptide, a protein, an oligonucleotide, a cell, an antibody, a positively or negatively charged oligopolymer, a molecularly imprinted polymer, an affinity polymer, and an organic or inorganic nanoparticle;
R¹ is selected from the group consisting of

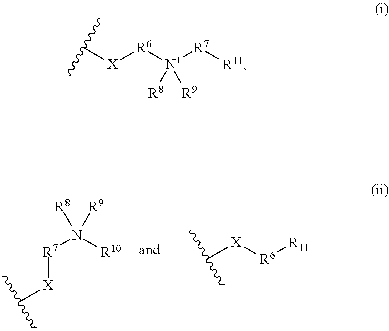

in a ratio of about 1:1 within the polymer;

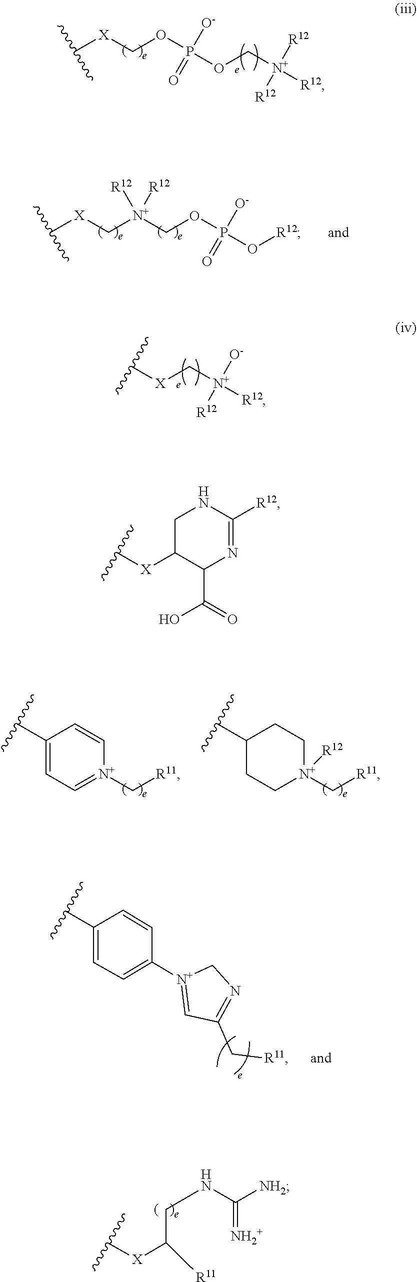

R² is

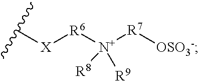

wherein
R⁶ and R⁷ are each independently selected from the group consisting of methyl, ethyl and propyl;
R⁸, R⁹, R¹⁰, R¹² and R¹³ are each methyl; and
R¹¹ is —SO₃⁻ or —C(═O)O⁻.

In an embodiment, the use of the present invention is one, wherein
R¹ is selected from the group consisting of

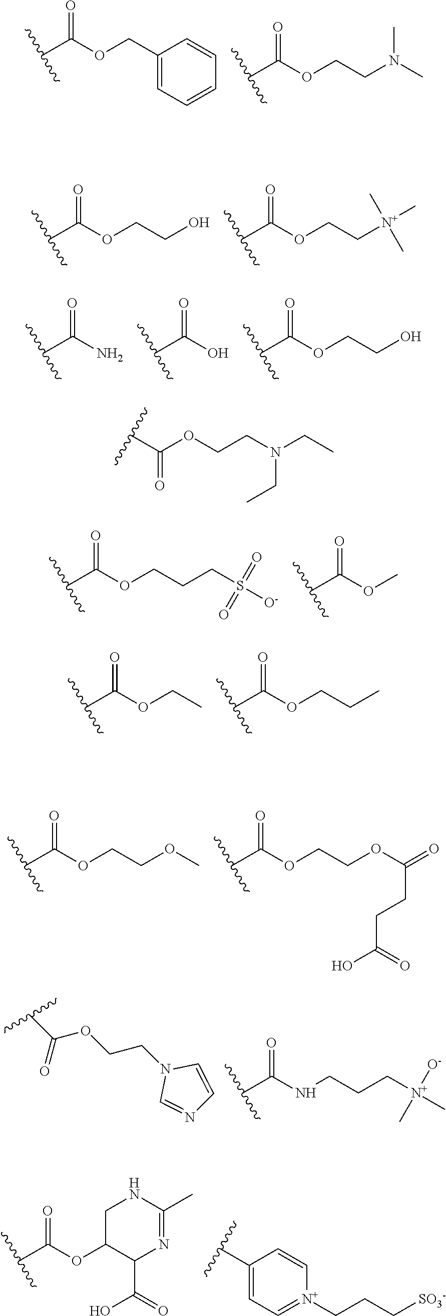

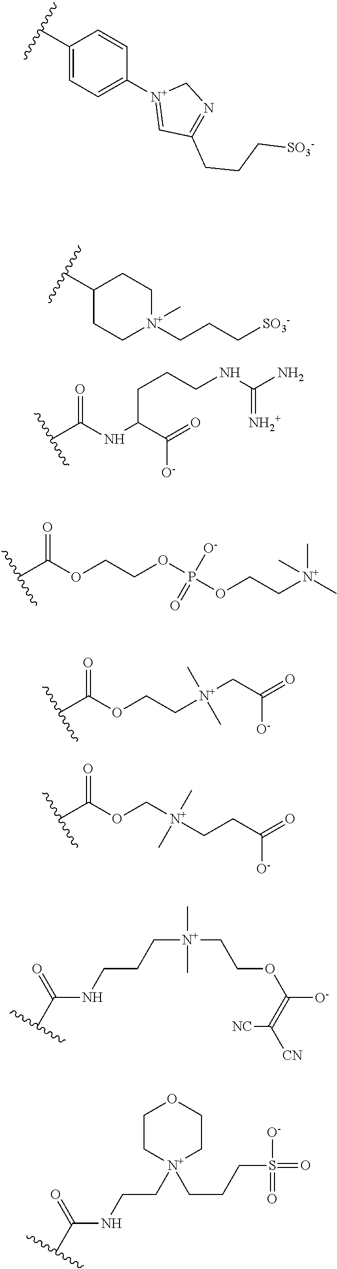

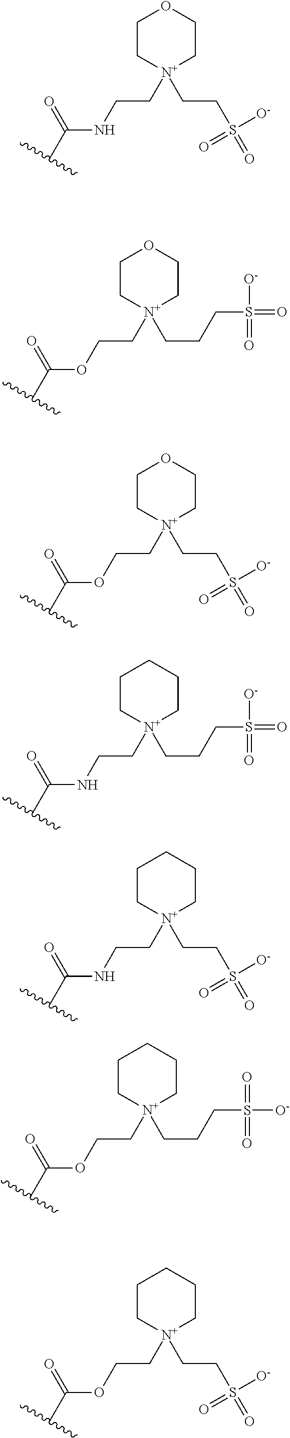

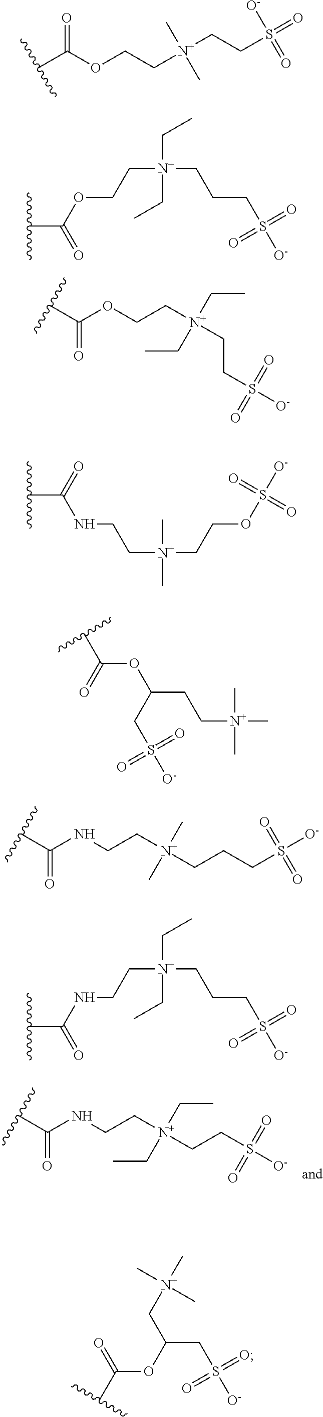

and
R² is selected from the group consisting of

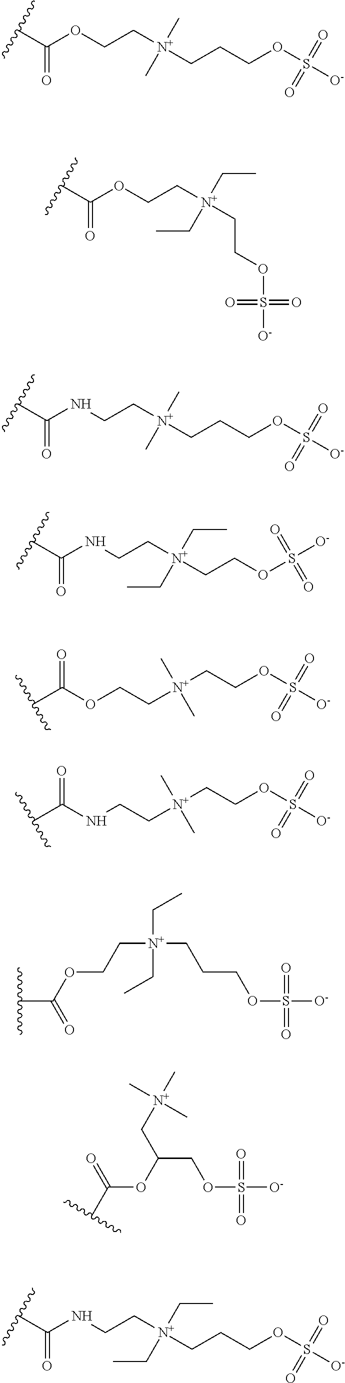

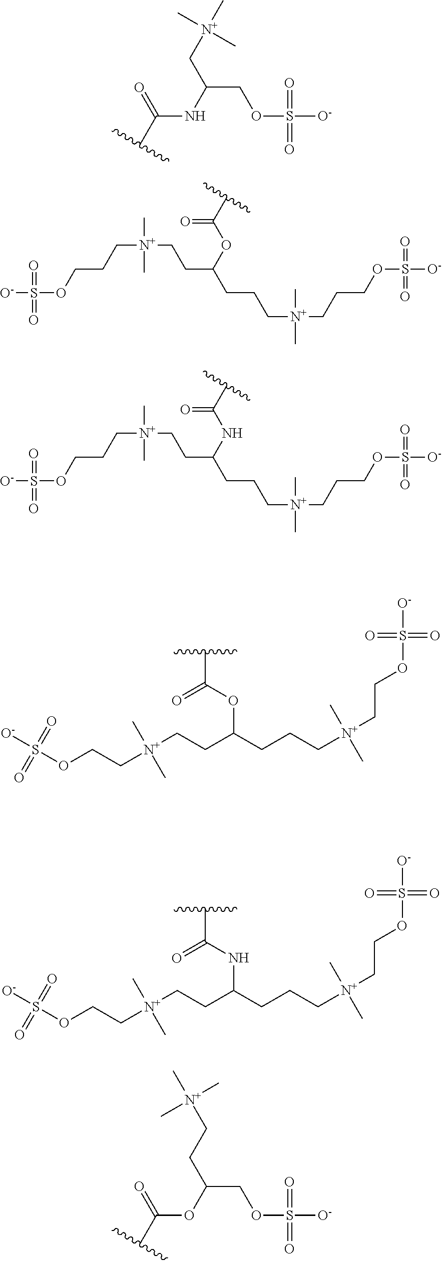

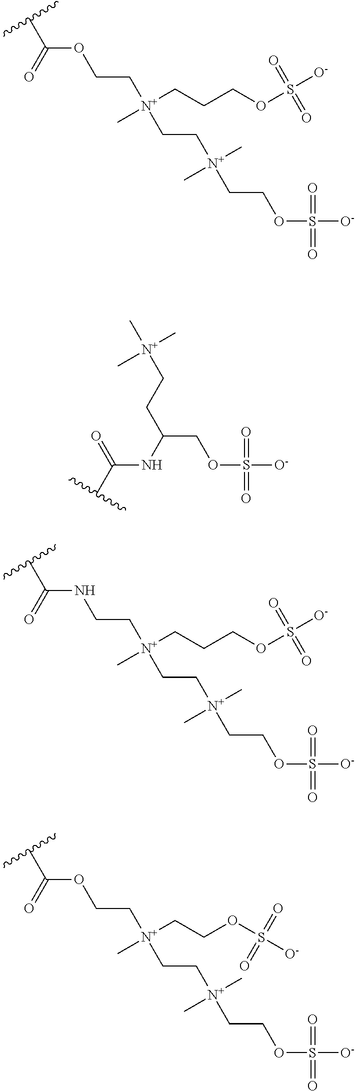 [SMILES: *C(=O)OCC[N+](C)(CCCOS(=O)(=O)[O-])CC[N+](C)(C)CCOS(=O)(=O)[O-] ; *C(=O)NC(CC[N+](C)(C)C)COS(=O)(=O)[O-] ; *C(=O)NCC[N+](C)(CCCOS(=O)(=O)[O-])CC[N+](C)(C)CCOS(=O)(=O)[O-] ; *C(=O)OCC[N+](C)(CCOS(=O)(=O)[O-])CC[N+](C)(C)CCOS(=O)(=O)[O-]]

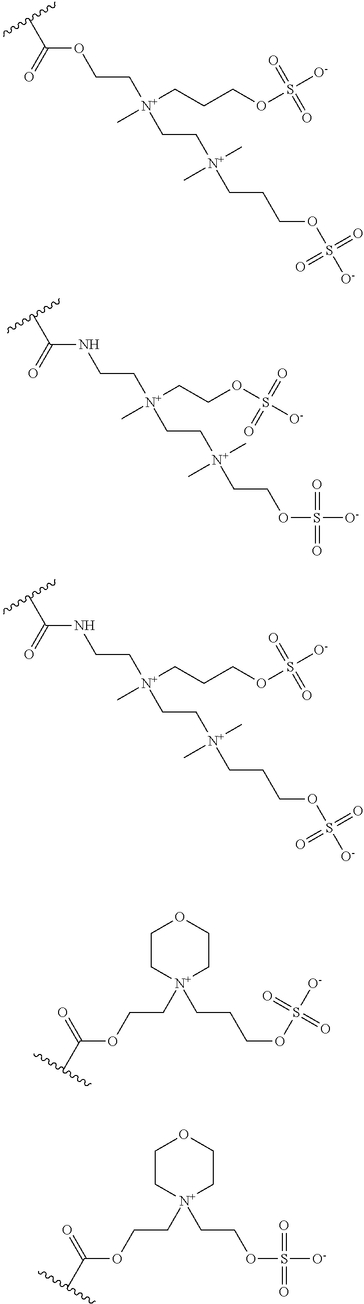 [SMILES: *C(=O)OCC[N+](C)(CCCOS(=O)(=O)[O-])CC[N+](C)(C)CCCOS(=O)(=O)[O-] ; *C(=O)NCC[N+](C)(CCOS(=O)(=O)[O-])CC[N+](C)(C)CCOS(=O)(=O)[O-] ; *C(=O)NCC[N+](C)(CCCOS(=O)(=O)[O-])CC[N+](C)(C)CCCOS(=O)(=O)[O-] ; *C(=O)OCC[N+]1(CCCOS(=O)(=O)[O-])CCOCC1 ; *C(=O)OCC[N+]1(CCOS(=O)(=O)[O-])CCOCC1]

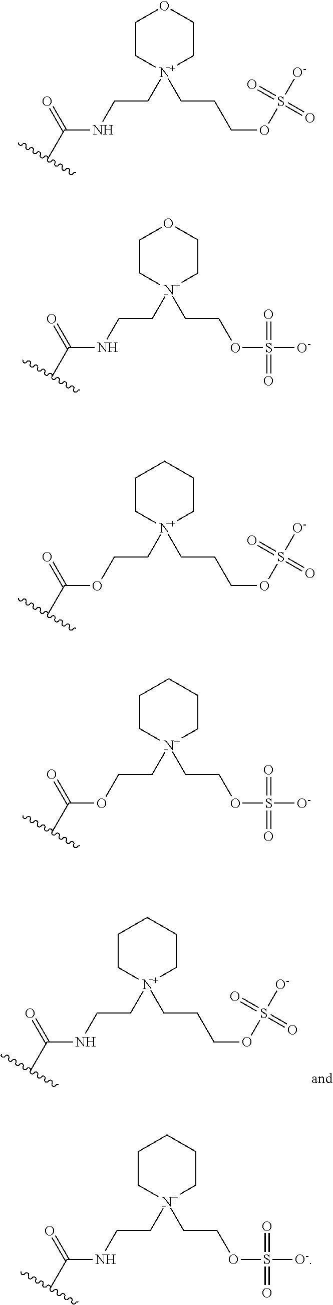

In an embodiment, the use of the present invention is one, wherein the polymer is selected from the group consisting of

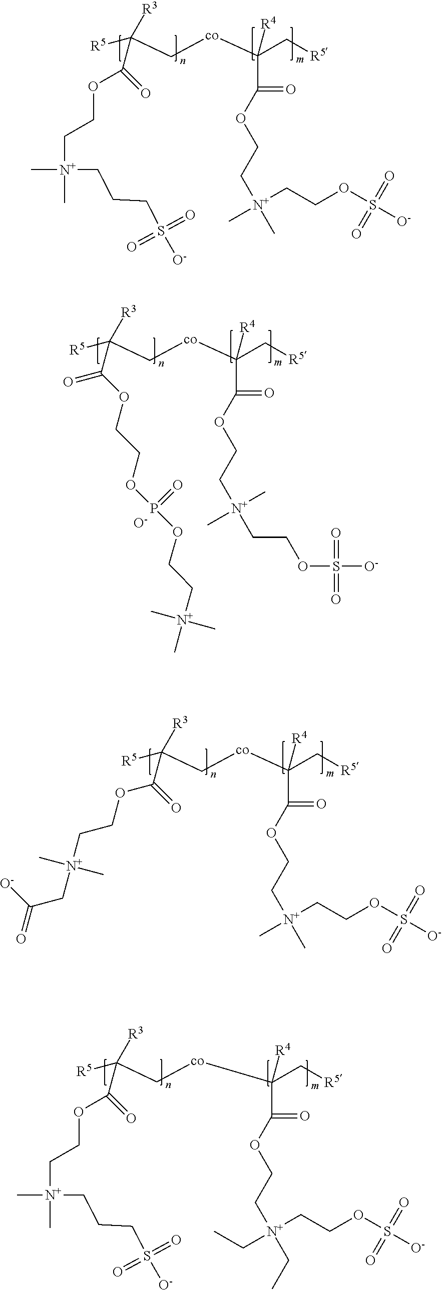

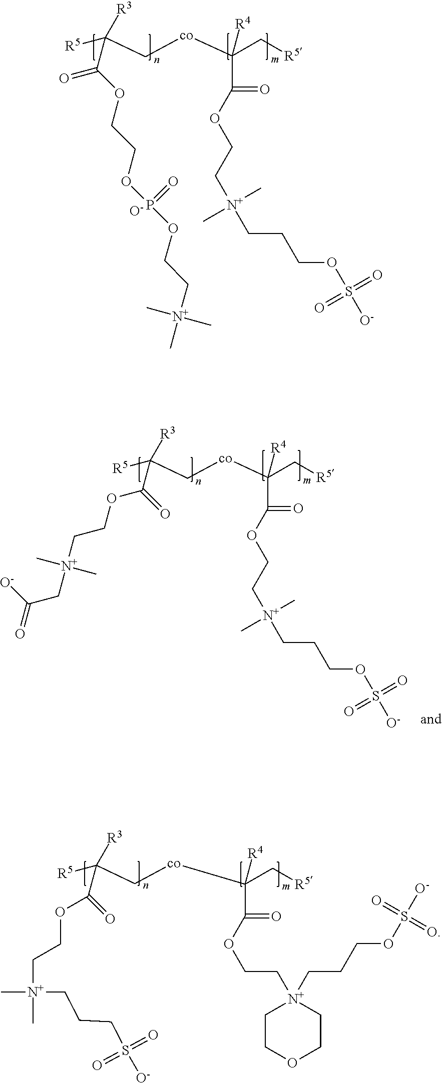

Optionally, R⁵ and R^{5′} of the above-noted polymers are selected from a group resulting from a RAFT polymerization initiator, optionally

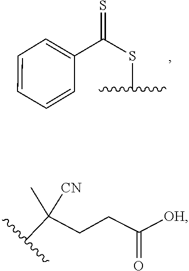

or a target binding moiety (TBM), optionally a TBM attached to or reacted with a group resulting from a polymerization initiator, optionally from a RAFT polymerization initiator, optionally

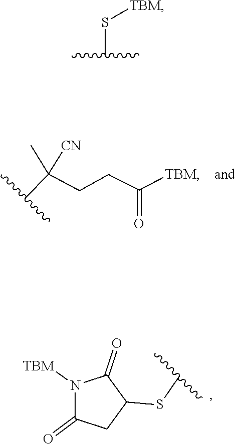

optionally

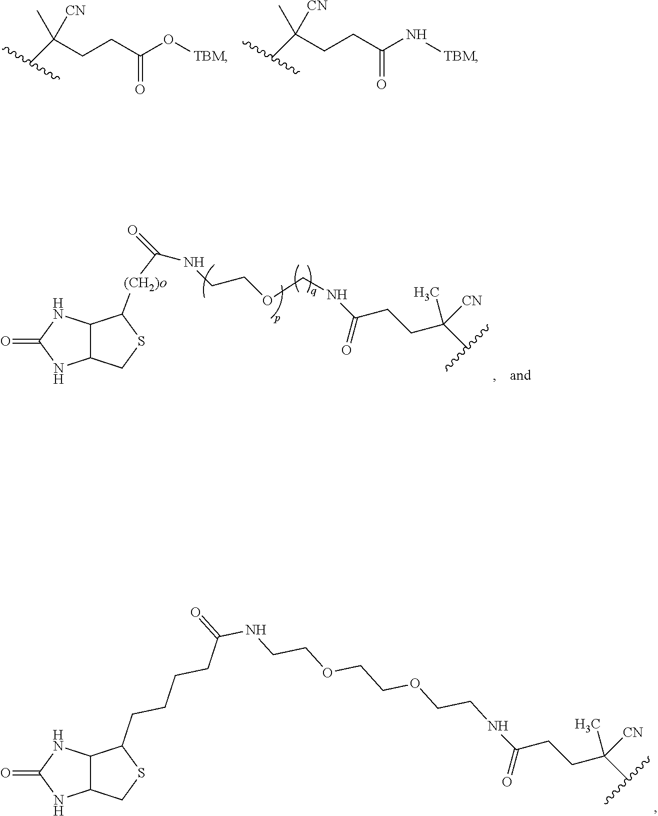

wherein o in (CH₂)o is an integer from 1 to 10, optionally 2 to 5, p is an integer from 1 to 5, optionally 2, and q is an integer from 1 to 5.

The exemplary combinations of R¹ and R² described herein in the context of the use are also meant to be specifically disclosed in all other aspects of the present invention, i.e. in the context of the polymer as such (with the limitations provided below) and in the context of the method.

In an embodiment, the use of the present invention is one, wherein the crosslinker is selected from the group consisting of

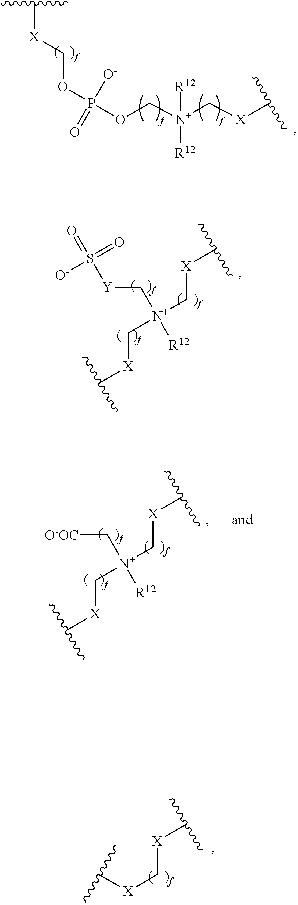

wherein
f is an integer selected from 1 to 5, optionally from 2 to 3;
X is selected from the group consisting of —C(═O)—O—, —C(═O)—NH—, aliphatic and aromatic carbo- or heterocycle, optionally —C(═O)—O—and —C(═O)—NH—; Y is oxygen or absent; and
R¹² is selected from the group consisting of methyl, ethyl and propyl;
optionally the crosslinker is selected from the group consisting of

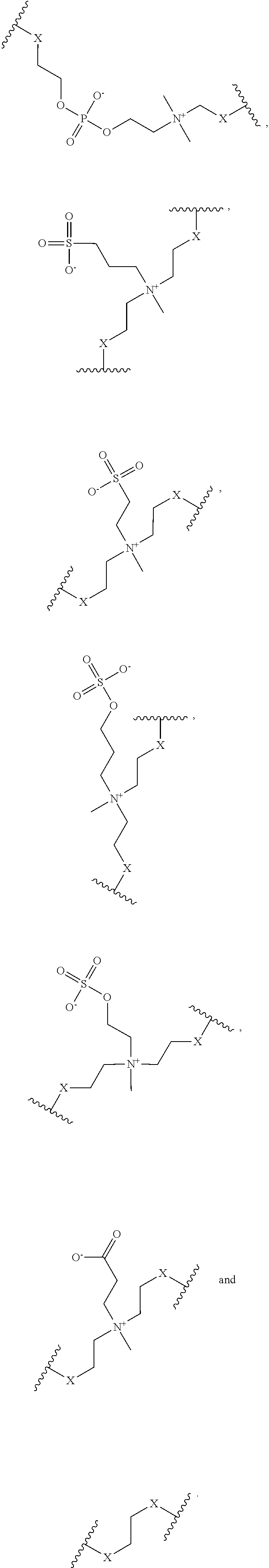

The crosslinker is attached to the backbone of the polymer at the position indicated with the wavy line. The crosslinker can, e.g., crosslink two R¹ or R² positions or an R¹ with an R² position, or an R² with an R¹ position in the polymer backbone.

In an embodiment, the use of the present invention is one, wherein
a. the polymer has a degree of polymerization (n+m) from 5 to 5000, optionally from 50 to 500; and/or
b. the percentage of n in the polymer ([n/(n+m)]*100) is in the range from 0 to 99.5%, optionally 0 to 70%, optionally 20 to 60%; and/or
c. the dispersity of the polymer is in the range of 1 to 5, optionally 1 to 1.5;
d. the crosslinker molar fraction in the polymer is in the range of 0 to 99%, optionally 0 to 5%; and/or
e. the droplets are responsive to temperature, shear, ionic strength, pH and/or a magnetic field.

The degree of polymerization and the dispersity can be determined by known methods. For example, nuclear magnetic resonance (¹H-NMR) can be used for determining the degree of polymerization and polymer dispersity and molecular weight can be evaluated via gel permeation chromatography.

Responsiveness to temperature, ionic strength, pH, shear and/or a magnetic field means, for example, that the droplets can be disassembled and/or assembled upon change of temperature, ionic strength and/or pH. The morphology of the droplets can be changed, e.g., by applying a magnetic field. Moreover, the surface tension, multiphase miscibility, size, polarity, viscosity, water content, and/or density of the droplets can be controlled, e.g., by changing the properties of the surrounding environment (e.g. temperature, ionic strength, pH, shear, and composition). This control and responsiveness also allows for the control of the uptake of compounds from outside to the inside of the liquid droplets as described above.

In an embodiment, the use of the present invention is one, wherein
a. the compounds for uptake, storage and/or reaction are selected from the group consisting of small molecules, drugs, antibodies, antigens, RNA, nucleic acids, viruses, carbohydrates, membrane-bound vesicles, contaminants, DNA, exosomes, extracellular vesicles, cells, proteins, peptides, biomolecules, enzymes, and ab42; and/or
b. the uptake is affinity- and/or binding-controlled, optionally mediated by interactions of the compound with R⁵ and/or R^{5′} of Formula (I).

In an embodiment, the use of the present invention is one, wherein the droplets are used
(i) for diagnosis of compounds, optionally of biomarkers,
(ii) for isolation for removal and/or enrichment of compounds, optionally for water treatment or cleaning,
(iii) for purification, extraction and/or separation of compounds,
(iv) for detoxification,
(v) for drug screening,
(vi) as cell culture scaffolds, and
(vii) in affinity assays.

The interactions of the compound with R⁵ and/or R^{5′} of Formula (I) as noted above means that the interaction can be with R⁵ and/or R^{5′} being or comprising a TBM as defined above.

In an embodiment, the use of the present invention is one, wherein the droplets comprise a polymer according to the formula

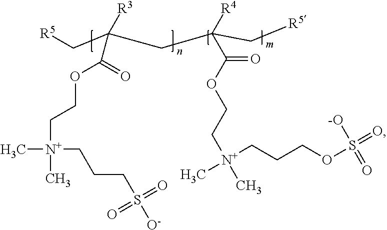

wherein m is an integer from 100 to 800, n is selected so that n/(n+m)*100 is between 0.125% and 50% and R^{5′} and/or R⁵ is a TBM, optionally a protein, a peptide, or an aptamer; or
wherein m is an integer from 70 to 100, n is selected so that n/(n+m)*100 is between 1% and 30% and R^{5′} and/or R⁵ is a TBM, optionally a protein, a peptide, or an aptamer.

In an embodiment, the use of the present invention is one, wherein the droplets comprise a polymer according to the formula

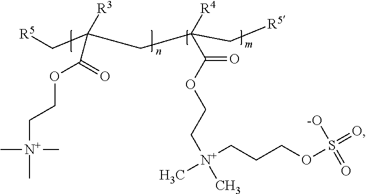

wherein m is an integer from 160 to 800, n is selected so that n/(n+m)*100 is between 0.125% and 40% and optionally R^{5′} and/or R⁵ is a TBM, optionally a protein, a peptide, or an aptamer; or
wherein m is an integer from 80 to 159, n is selected so that n/(n+m)*100 is between 0.625% and 20% and optionally R^{5′} and/or R⁵ is a TBM, optionally a protein, a peptide, or an aptamer.

In an embodiment, the use of the present invention is one, wherein the droplets comprise a polymer according to the formula

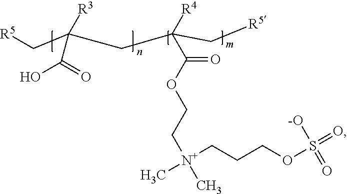

wherein m is an integer from 80 to 800, n is selected so that n/(n+m)*100 is between 0.125% and 20% and optionally R^{5′} and/or R⁵ is a TBM, optionally a protein, a peptide, or an aptamer.

In an embodiment, the use of the present invention is one, wherein the droplets comprise a polymer according to the formula

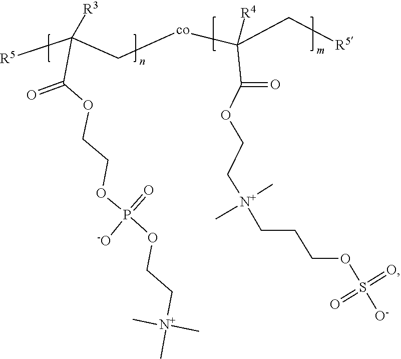

wherein m is an integer from 80 to 800, n is selected so that n/(n+m)*100 is between 0.125% and 15% and R^{5′} and/or R⁵ is a TBM, optionally a protein, a peptide, or an aptamer.

In an embodiment, the use of the present invention is one, wherein the droplets comprise a polymer, wherein R¹ is

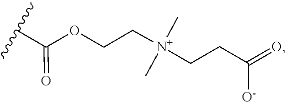 [SMILES: *C(=O)OCC[N+](C)(C)CCC(=O)[O-]]

### R² is

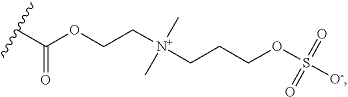

m is an integer from 160 to 800, n is selected so that n/(n+m)*100 is between 0.125% and 20% and R^{5′} and/or R⁵ is a TBM, optionally a protein, a peptide, or an aptamer.

In an embodiment, the use of the present invention is one, wherein the droplets comprise a polymer, wherein R¹ is

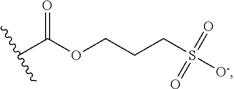

### R² is

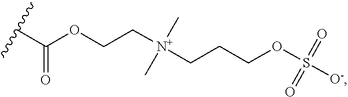

m is an integer from 80 to 800, n is selected so that n/(n+m)*100 is between 0.125% and 20% and optionally R^{5′} and/or R⁵ is a TBM, optionally a protein, a peptide, or an aptamer.

In an embodiment, the use of the present invention is one, wherein the droplets comprise a polymer, wherein R¹ is

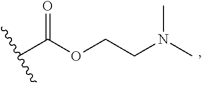 [SMILES: *C(=O)OCCN(C)C]

### R² is

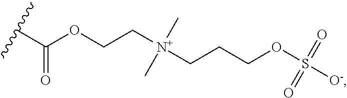

m is an integer from 80 to 800, n is selected so that n/(n+m)*100 is between 0.125% and 20% and optionally R^{5′} and/or R⁵ is a TBM, optionally a protein, a peptide, or an aptamer.

In an embodiment, the use of the present invention is one, wherein the droplets comprise a polymer, wherein R¹ is a hydrophobic residue, optionally

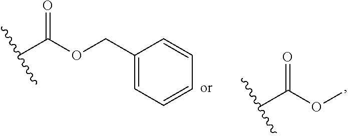

### R² is

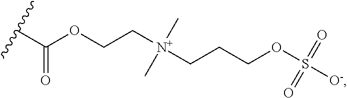

m is an integer from 1 to 900, n is optionally selected so that n/(n+m)*100 is between 0.125% and 20% and optionally R^{5′} and/or R⁵ is a TBM, optionally a protein, a peptide, or an aptamer.

In another aspect the present invention is directed to a polymer for forming liquid droplets in an aqueous medium, wherein the polymer is a polymer as defined herein with the proviso that
(i) R¹ is not

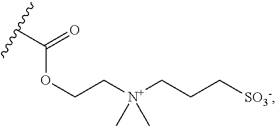

and/or
(ii) R⁵ and R^{5′} are not

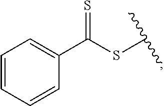

if R² is

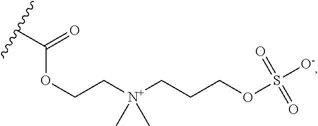

and wherein the droplets take up and/or store compounds in an aqueous medium.

In an embodiment, the polymer of the present invention is a polymer as defined herein with the proviso that R⁵ is not bromine if n=0. In another embodiment, the polymer of the present invention is a polymer as defined herein with the proviso that at least one of R⁵ and R^{5′} is a TBM if n=0. In another embodiment, the polymer of the present invention is a polymer as defined herein with the proviso that at least one of R⁵ and R^{5′} is a group resulting from a RAFT polymerization initiator if n=0.

The polymer of the present invention for forming liquid droplets in an aqueous medium can be any polymer described herein in the context of the use and the method as long as the above conditions for R¹, R⁵, R^{5′} and/or R² are met.

All properties and parameters described herein for the polymer in the context of the use can optionally also apply to the polymer for forming liquid droplets in an aqueous medium and to the corresponding method for production, e.g. parameters and properties described herein directed to the degree of polymerization, the percentage of n in the polymer, the dispersity of the polymer, molar fraction of crosslinker and/or the factors to which the polymers are responsive to.

In an embodiment, the polymer of the present invention is one, wherein R¹ is selected from the group consisting of

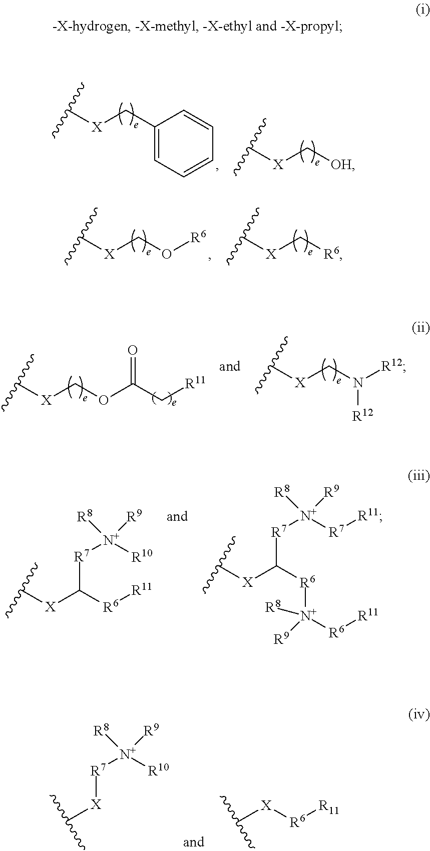

in a ratio of about 1:1 within the polymer;

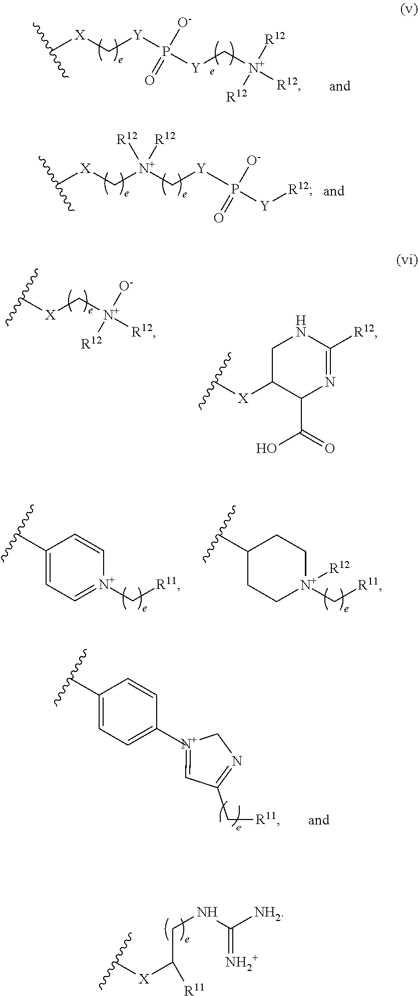

In an embodiment, the polymer of the present invention is one, wherein
R¹ is selected from the group consisting of

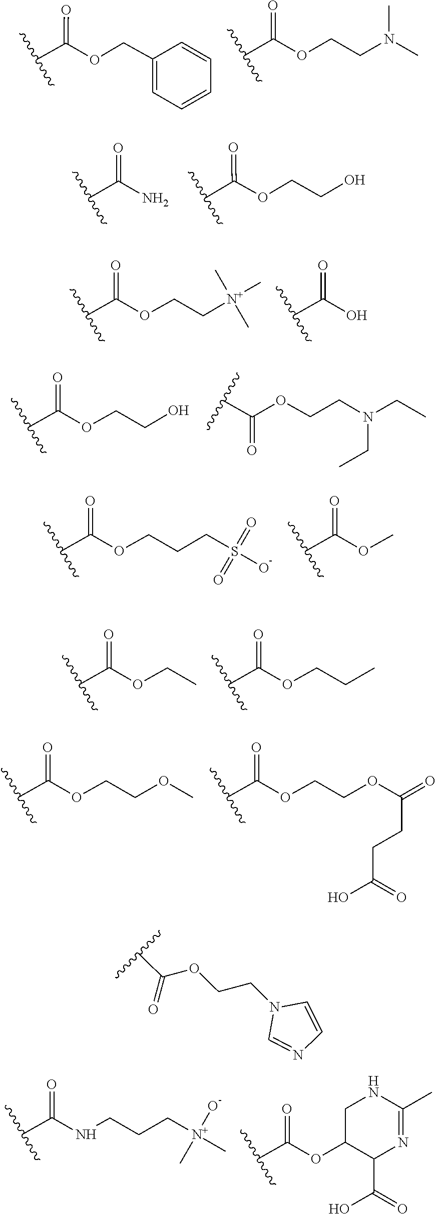

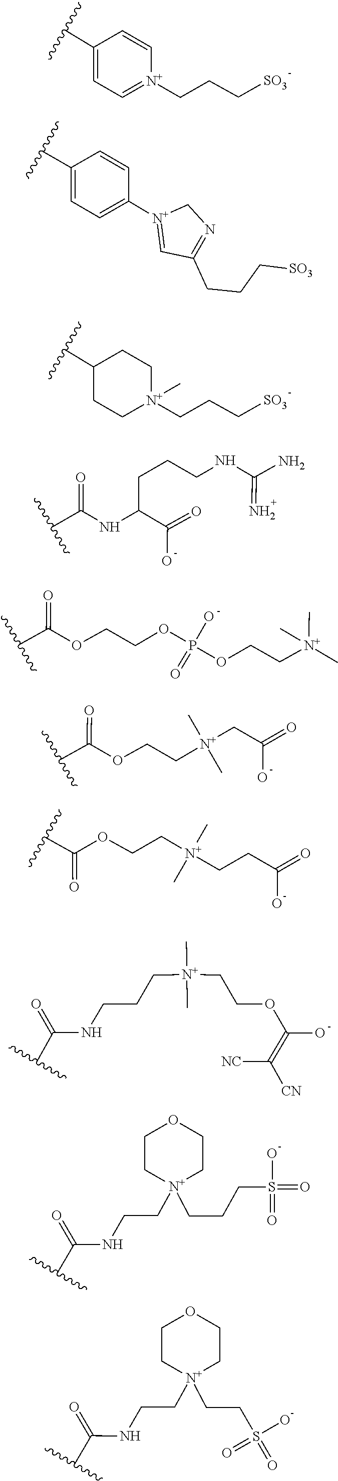

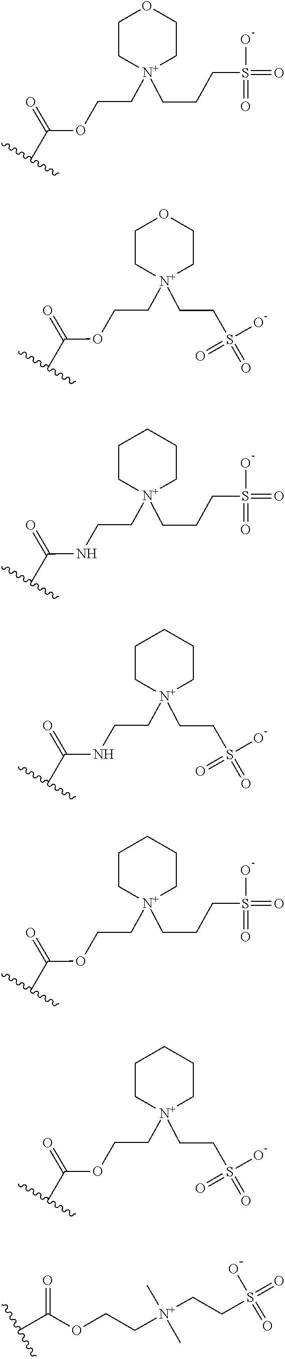

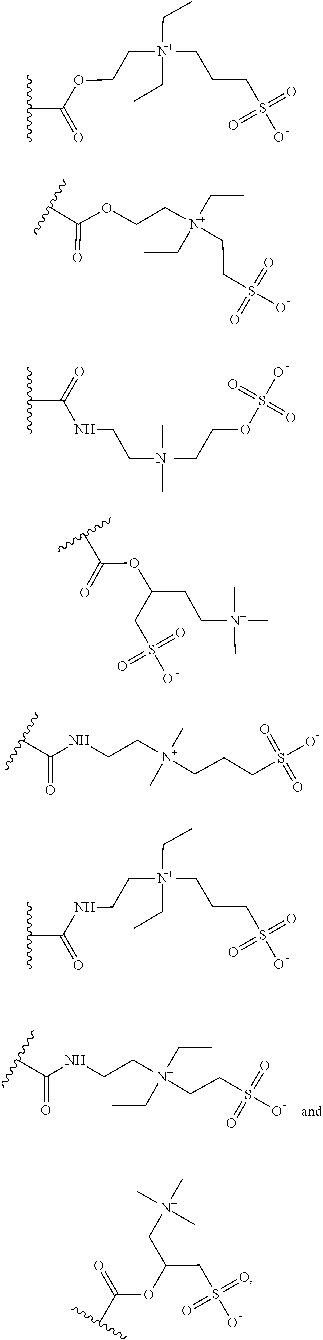

and/or
R² is selected from the group consisting of

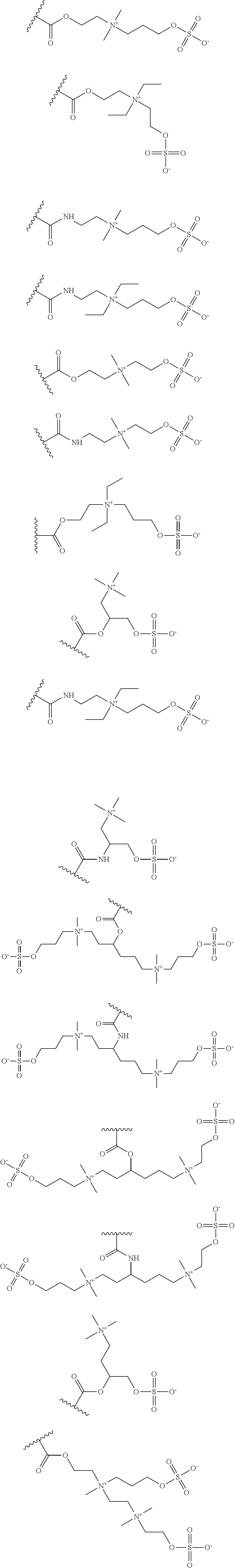

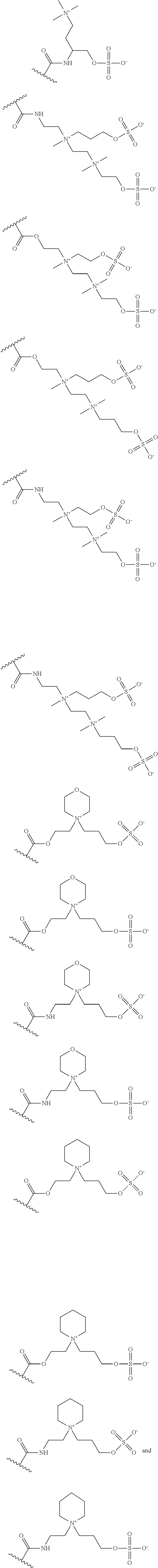

In another aspect, the present invention is directed to a method for producing a polymer for forming liquid droplets in an aqueous medium as described herein comprising the following steps:
(a) providing monomers selected from the group consisting of
   methyl methacrylate, ethyl methacrylate, propyl methacrylate, butyl methacrylate, hydroxyethyl methacrylate (HEMA), methacrylamide (Mam), benzyl methacrylate (Be), 2-dimethylaminoethyl methacrylate methyl chloride (MQ) and 2-dimethylaminoethyl methacrylate (DMAEMA),

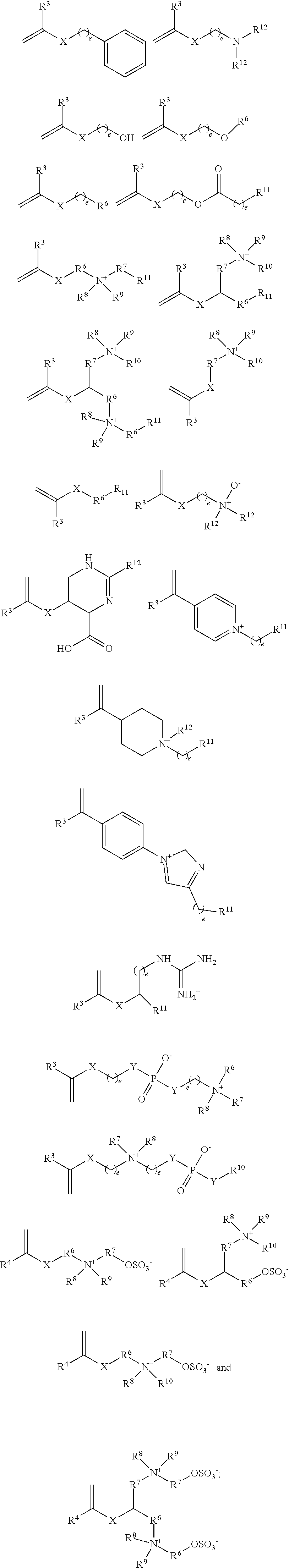

(b) optionally providing crosslinker monomers, optionally selected from the group consisting of

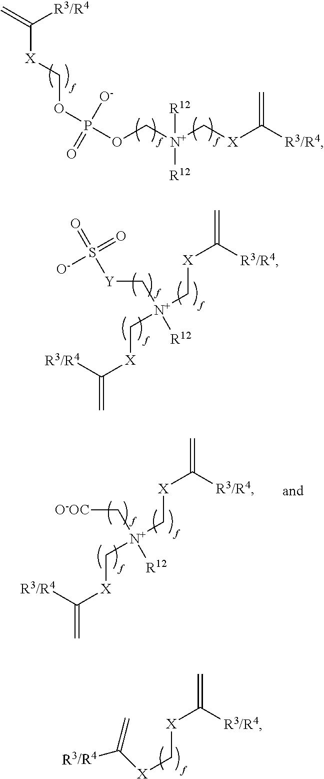

(c) optionally in a molar fraction in the range of 0 to 99%, optionally 0 to 5%;
(d) reacting the monomers of step (a) and optionally (b) in a free radical polymerization or in a controlled radical polymerization, optionally using RAFT, ATRP or NMP in a suitable solvent;
(e) optionally functionalizing the polymer with R⁵ and/or R^{5′}, optionally in an esterification reaction, by click chemistry, amidation, thioesterification or by non-covalent modification; and
(f) isolating and optionally purifying the polymer.

The monomers for use in the present method can be any monomer as defined above or any monomer that corresponds to a residue defined for R¹ and R² in the context of the polymer for forming liquid droplets in an aqueous medium described herein. A monomer corresponding to a residue defined for R¹ and R² is a monomer wherein the entity

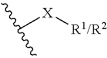

is replaced by

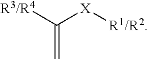

For example, the present invention also encompasses the method described herein for producing a polymer described herein in the context of the use of the polymer. For this method, the monomers used correspond to monomers which result in a residue defined for R¹ and R² in the context of the use of the polymer.

Reacting the monomers of step (a) and optionally (b) of the present method can also be achieved, e.g., using redox initiators or photoinitiators (for example, in combination with a UV source).

In step (c) of the present method, the suitable solvent may be an aqueous solvent, optionally a buffer selected from acetic buffer, aqueous buffer, mixtures of aqueous buffer with a water-miscible organic solvent, optionally ethanol, DMSO, DMF and THF, optionally a buffer with a salt concentration of about 500 mM to 3 M NaCl. Step (c) may optionally be performed in the absence of oxygen and presence of heat, optionally from 50 to 80° C.

Functionalizing the polymer with R⁵ and/or R^{5′}, is to be understood as described in the context of the use described herein. R⁵ and/or R^{5′} may be a chemical entity resulting from a polymerization reaction as described herein, which entity is further chemically modified or functionalized (e.g. reacted with a TBM) to be a target binding moiety; or R⁵ and/or R^{5′} may be a target binding moiety in the absence of a chemical entity resulting from a polymerization. The term “functionalizing” means that a TBM is attached to the group by means of a chemical reaction to form, e.g. a covalent or ionic, bond with the chemical entity resulting from a polymerization. This attachment may result in the direct attachment of the TBM to the polymer under loss of the chemical entity resulting from a polymerization or it may result in the chemical modification of the chemical entity, e.g. partial loss of chemical entity. In step (d) of the present method, the following exemplary and non-limiting reactions for functionalizing the polymer can be made:

For an esterification between a carboxylic acid residue in the polymer with an amine-bearing targeting agent (e.g. antibody, antigen, protein A), the esterification can be performed in an aqueous buffer with EDC/NHS coupling.

For an azide-alkyne cycloaddition between an alkyne bearing polymer and an azide-bearing targeting agent such as, e.g. Biotin-azide (e.g. PEG4 carboxamide-6-azydoexanyl biotin), the reaction is not catalyzed by copper if DBCO is used as alkyne.

For a Michael addition between a thiol generated by the aminolysis of the carbonylthiol group of the RAFT agent and an alkene or maleimide-containing targeting agent (e.g. Biotin-maleimide), reference is made to H. Willcock et al. Polym. Chem., 2010, 1, 149-157.

The step of isolating and purifying in the present method can be done, e.g., via either dialysis or diafiltration against an aqueous buffer, via precipitation in a mixture of an aqueous buffer and organic solvent or via precipitation in an organic solvent.

In another aspect, the present invention is directed to a polymer for forming liquid droplets in an aqueous medium as described herein obtained or obtainable by the process described herein.

In an embodiment, the monomers described herein, including the monomer as defined above or any monomer that corresponds to a residue defined for R¹ and R² in the context of the polymer for forming liquid droplets in an aqueous medium as described herein, are polymerized to obtain the polymer for forming liquid droplets as described in any aspect of this disclosure.

In an embodiment, monomer(s) comprising residue R¹ and monomer(s) comprising residue R² are polymerized in a ratio of 0:1 to 100:1, optionally 0:1 to 10:1 or 0:1 to 1:1.

### BRIEF DESCRIPTION OF THE DRAWINGS

The following Figures and Examples serve to illustrate the invention and are not intended to limit the scope of the invention as described in the appended claims.

FIGS. 1A, 1B, 1C, 1D, and 1E. FIG. 1A. Depicts a schematic illustration of the strategy of the invention. FIG. 1B. Formation of ZW coacervates as a function of temperature, as detected by measuring the average hydrodynamic radius by dynamic light scattering of 0.5 mg mL⁻¹ ZW2 aqueous solution at 30 mM NaCl and pH=7.5. Phase diagram of ZW2 at different polymer concentrations and ionic strengths. Red cross and black circles indicate absence and presence of phase separation. FIG. 1C. Hydrodynamic size of EG droplets as function of temperature. 0.5 mg mL⁻¹ EG in distilled water. Phase diagram of EG at different polymer concentrations and ionic strengths. Cross and circles indicate absence and presence of phase separation. FIG. 1D. Fusion of ZW2 droplets and (non fusion) of EG coacervates within microfluidic water-in-oil compartments. FIG. 1E. Fluorescence recovery after photobleaching (FRAP) of ZW2 and EG coacervates. ZW in the figures refers to ZW2.

FIGS. 2A, 2B, 2C, 2D, 2E, and 2F. FIG. 1A. Structure of the synthesized polymers encoding a progressively increasing number of interactions. FIG. 2B. Partition coefficient of different molecules into coacervates of different copolymers and representative confocal images. FIG. 2C. Partition coefficient of Rhodamine and BSA in coacervates of different copolymers. FIG. 2D. Relative polarity with respect to water of the compartments made of different copolymers. FIG. 2E. Modulation of the cloud point temperature by modulating the composition of ZW and ZWH copolymers. FIG. 2F. Changes in the partition coefficient of Rhodamine by modulating the composition of ZW and ZWH copolymers. (ZW and ZWH refers to ZW2 and ZWH1, respectively).

FIGS. 3A, 3B, and 3C. Programmable droplets for bioseparation. FIG. 3A. Confocal images showing the recruitment of fluorescent streptavidin in ZW10-biotin droplets (ZW-Biotin), IgG in ZW10-Protein A (ZW-Protein A) droplets and liposomes into ZW11-streptavidin (ZW-Strep) droplets (upper panels). Lower panels show controls with compartments based on non-functionalized ZW polymers, showing the absence of uptake. FIG. 3B. Isolation of IgG molecules from a mixture of multiple proteins (bovine serum albumin (BSA), the enzyme adenylate kinase (AK) and insulin) and from a cell lysate with droplets of ZW11 coupled to Protein A. FIG. 3C. Detection assay implemented in a droplet-microfluidic platform to quantify tiny amounts of streptavidin in fetal bovine serum with the ZW10-biotin based droplets (pDrop).

FIGS. 4A, 4B, and 4C. FIG. 4A. The liquid-liquid phase separation (LLPS) behavior of the ZW polymer changes upon binding to target analytes. For instance, the binding of the functionalized ZW to hydrophilic targets increases the solubility of the complexed ZW. If the uncomplexed polymer has a relatively low separating power, this binding leads to the migration of bound complexes outside the droplets, resulting in the shrinkage of the droplets and the reduction in the volume of the dispersed ZW phase. FIG. 4B. The shrinkage depends on the amount of hydrophilic target present. Using the change of solubility of ZW upon target binding, we can quantify the amount of target in solution, or the binding affinity of the conjugated ZW binding moiety and target. FIG. 4C. Other properties of the ZW change upon target binding, such as the Tcp which will decrease, and can also be monitored for the same applications suggested in b).

FIGS. 5A, 5B, 5C, and 5D. Multiphase immiscible droplets. Depending on the interfacial energy, compartments of two different polymers can be fully miscible (ZW2/ZW3 pair) (FIG. 5A), fully immiscible (EG/ZW2 pair) (FIG. 5B) or form subcompartments, either with a core-shell morphology (ZWBe/ZW3 pair) (FIG. 5C) or with partial wetting (ZW2/ZWBe pair) (FIG. 5D). The coexistence of multiple immiscible droplets with different physiochemical properties and with different responsiveness to external stimuli can be used to selectively recruit, purify, and detect different solutes at the same time from a single complex mixture.

FIGS. 6A, 6B, 6C, 6D, 6E, 6F, and 6G. FIG. 6A. Uptake of proteins, DNA and CD81+ extracellular vesicles (EV) into ZW+8 droplets at different salt concentrations. A low amount in solution indicates high uptake. FIG. 6B. Uptake of liposomes into droplets of ZW+8 (i.e. random polymer) and ZW+9 (i.e. block copolymer). The distribution of the positive charges in the polymer sequence affects the salt concentration at which the liposomes can be recruited in the droplets. FIG. 6C. Fluorescence microscopy images showing the recruitment of liposomes in droplets of ZW+8 (i.e. random polymer) and ZW+9 (i.e. block polymer). The polymer architecture determines where the liposomes are recruited, either in the rim of the droplet or in the droplet bulk. FIG. 6D. Fluorescence microscopy images showing the impact of liposome concentration on ZW+8 droplets. The droplets dynamically adapt to the amount of liposomes in solution. At higher concentrations, more polymer interacts with the liposomes and droplet diameters decrease. FIG. 6E. Amount of CD81+ EVs that are recruited and released from ZW+8 droplets. CD81+ EVs are largely recruited into the droplets after different incubation conditions (1-15 min, 4° C.-25° C.). A significant fraction of CD81+ EVs are released from the droplets upon an increase in salt concentration. FIG. 6F. The size distribution of CD81+ EVs before and after being separated with ZW+8 droplets. FIG. 6G. TEM image showing the presence and the vesicle-like structure of EVs separated with ZW+8 droplets.

FIGS. 7A and 7B. The interaction properties of the droplets with different materials can be controlled by changing the polymer architecture. FIG. 7A. Fluorescence microscopy image showing that droplets of ZW+12, i.e. a positively charged ZW-based polymer with a short terminal hydrophilic block, wet the bottom of the plate. FIG. 7B. Fluorescence microscopy images showing that ZW+13, i.e. a positively charged ZW-based polymer with a long terminal hydrophilic block, forms droplets that do not interact with the surface and that are stable in time.

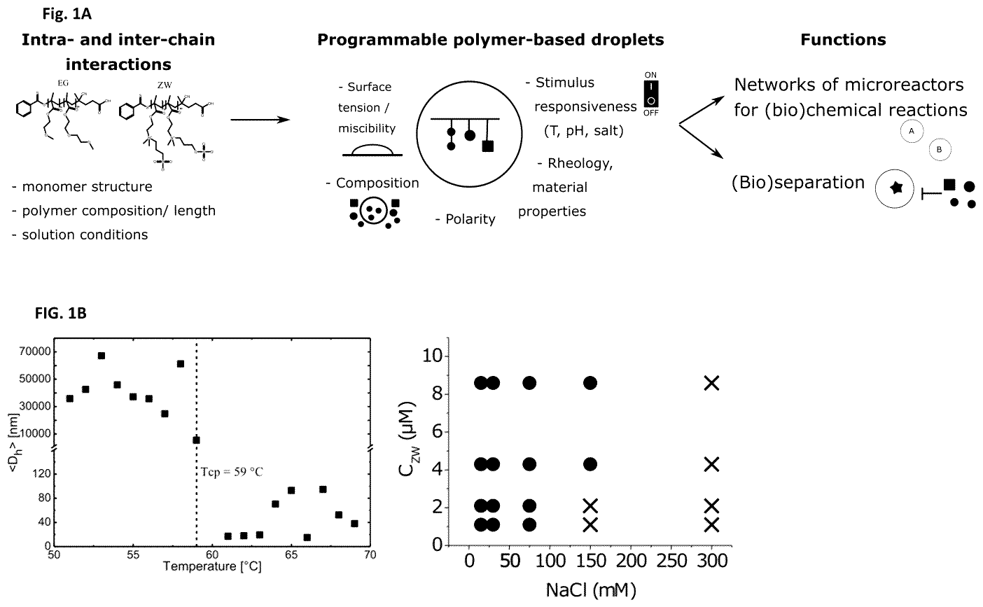

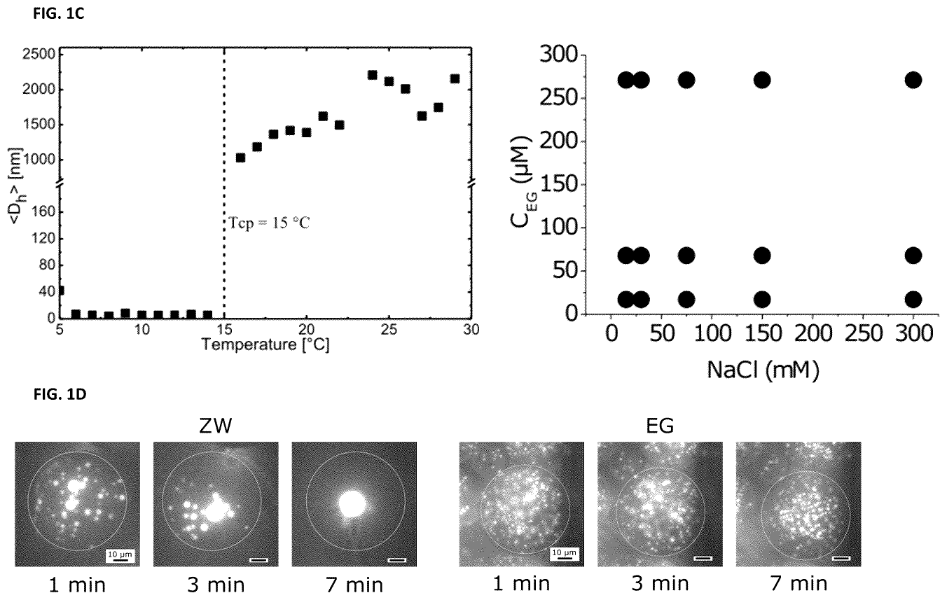

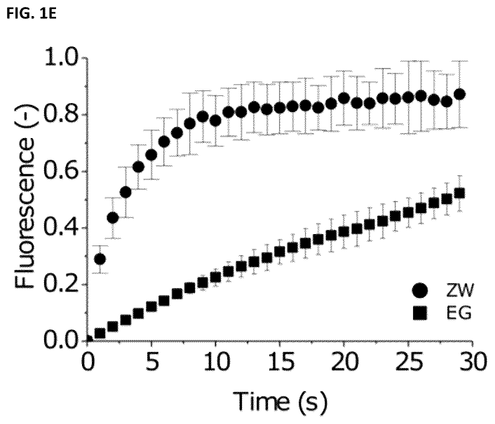

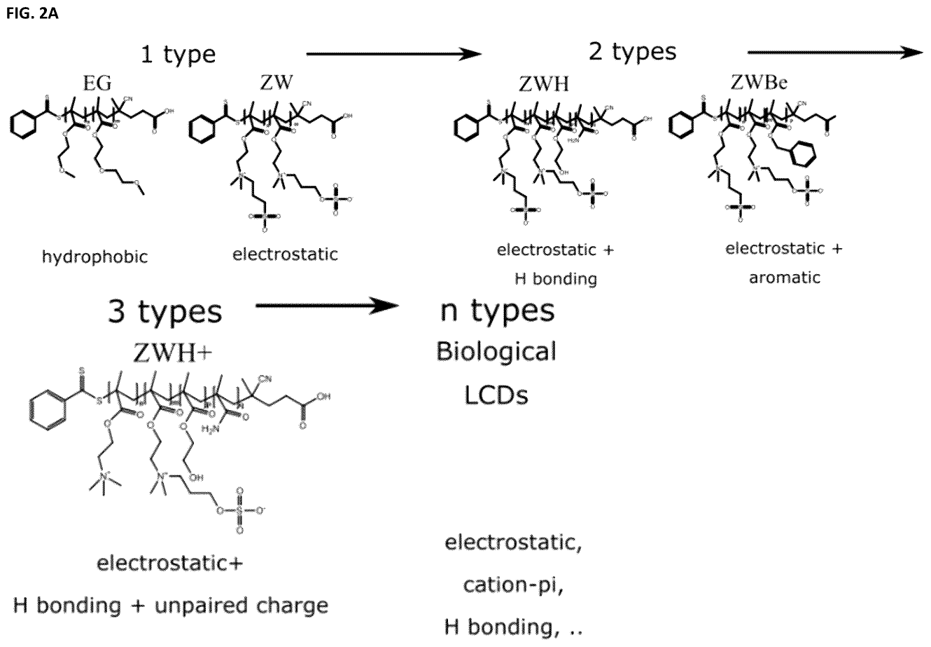

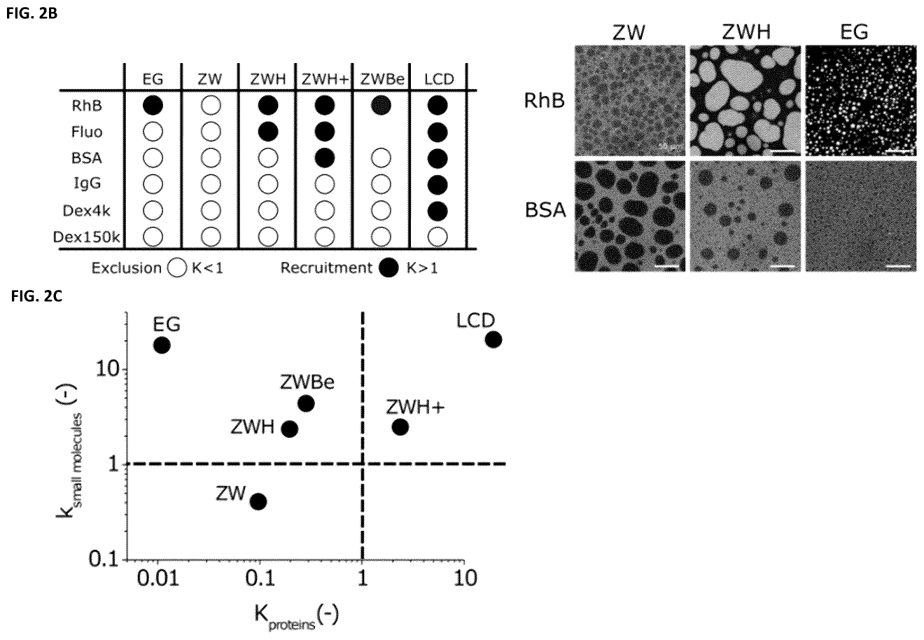

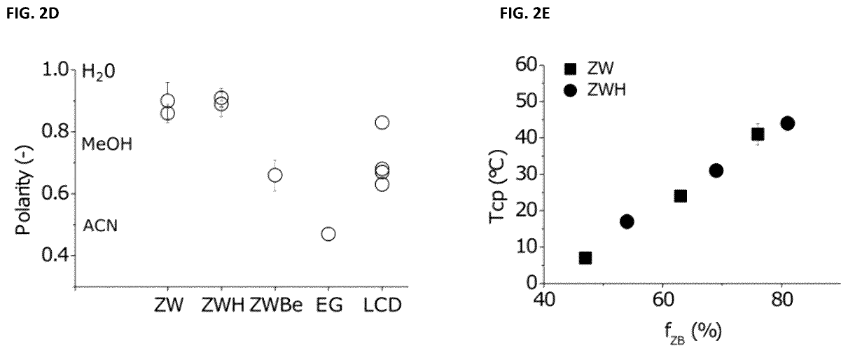

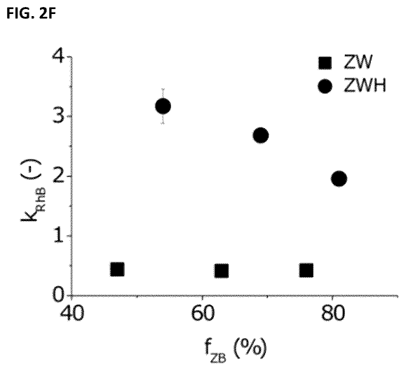

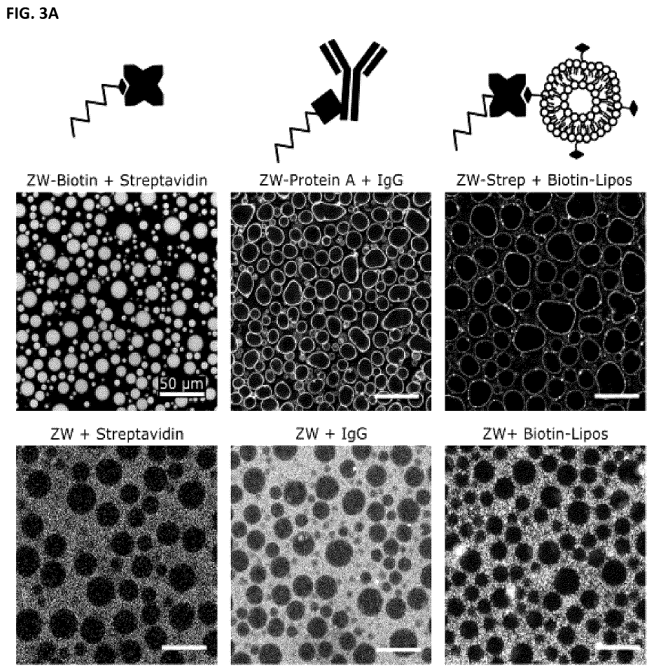

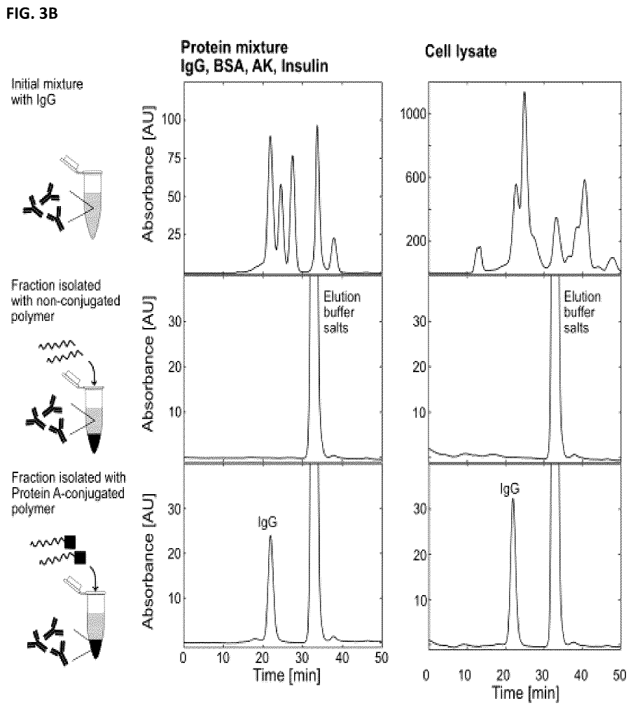

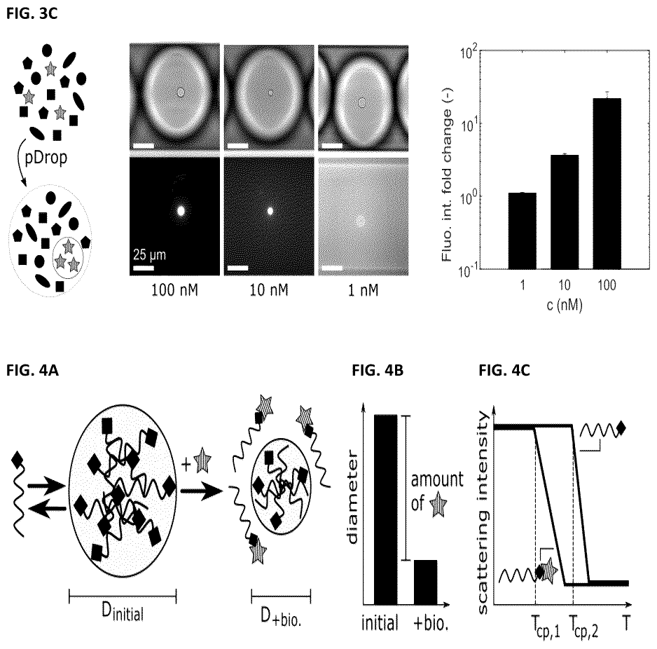

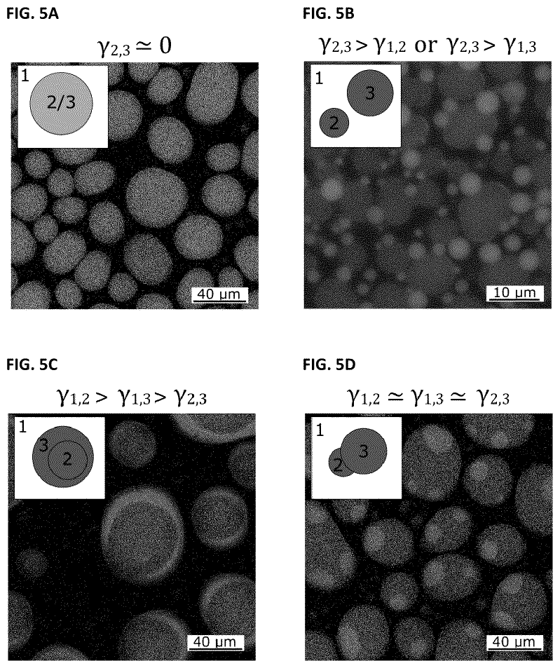

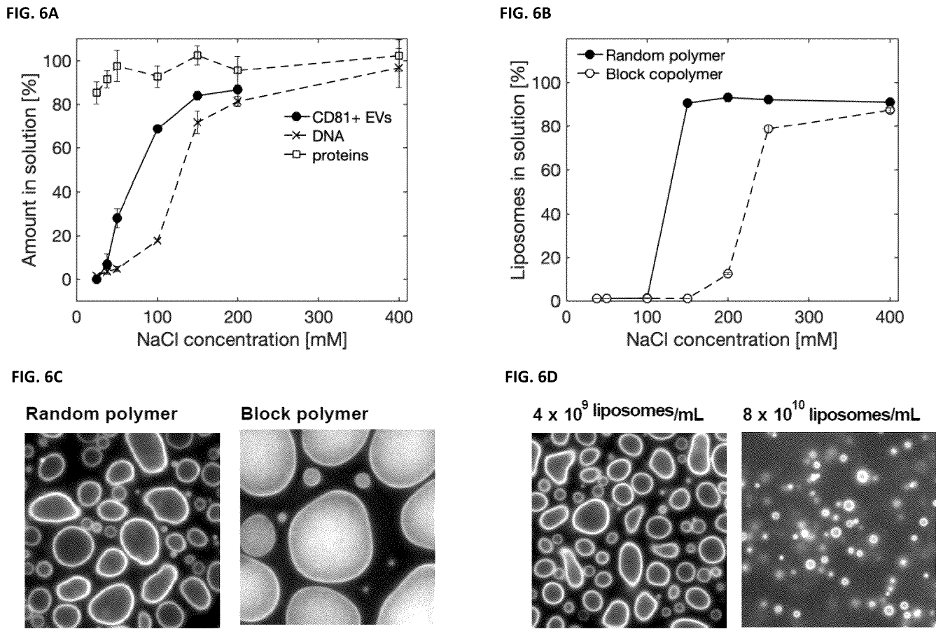

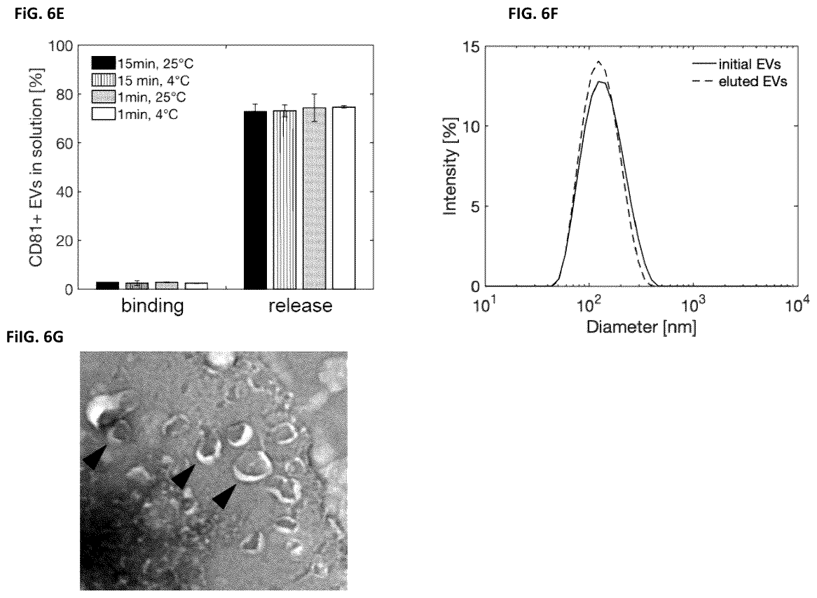

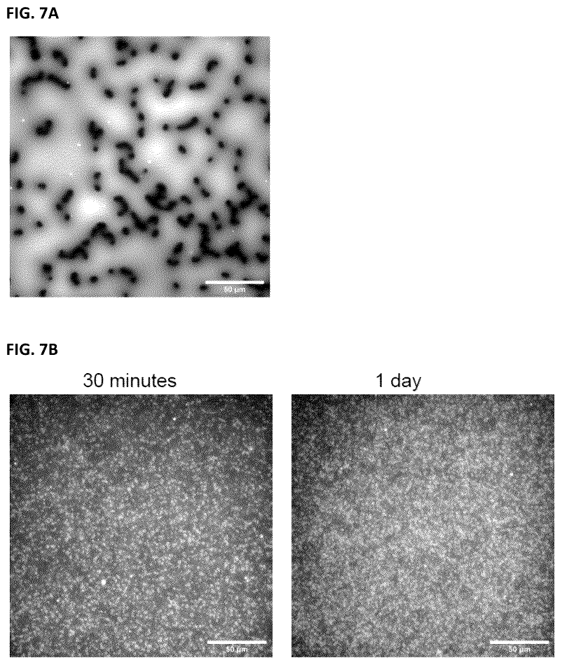

### DETAILED DESCRIPTION

### Example 1—Materials

Methacrylamide (Mam, 98%, MW=85.10, Sigma Aldrich), 2-hydroxyethyl methacrylate (HEMA, 97%, MW=130.14, Sigma Aldrich), 4,4′-azobis(4-cyanovaleric acid) (ACVA, >98%, MW=280.28, Sigma Aldrich), 4-cyano-4-(phenylcarbonothioylthio)pentanoic acid (CPA, >97%, MW=279.38, Sigma Aldrich), di(ethylene glycol) methyl ether methacrylate (95%, MW=188.22, Sigma Aldrich), ethylene glycol methyl ether methacrylate (95%, MW=144.17, Sigma Aldrich), N-(3-sulfopropyl)-N-methacroyloxyethyl-N,N-dimethylammonium betaine (SB, MW=279.35, Merck), ethanol (99.8%, MW=46.07, Sigma Aldrich), acetonitrile (ACN, 99.99%, MW=41.05, Fisher Chemicals), 1,3-propanediol cyclic sulfate (TMS, 98%, MW=138.14, Sigma Aldrich), sodium acetate (99%, MW=82.03, Sigma Aldrich), benzyl methacrylate (Be, 96%, MW=176.21 Sigma Aldrich), mono-2-(Methacryloyloxy)ethyl succinate (HSucc, 95%, MW=230.21, Sigma Aldrich), 2-dimethylaminoethyl methacrylate (DMAEMA, 98%, MW=157.21, Sigma Aldrich), methacrylic acid (MA, 99%, MW=86.09, Sigma Aldrich), 3-Sulfopropyl methacrylate potassium salt (SPMAK, 98%, MW=246.32, Sigma Aldrich), [2-(methacryloyloxy)ethyl]trimethylammonium chloride solution (MQ, 75% in H₂O, MW=207.7 g/mol, Sigma Aldrich), 2-methacryloyloxyethil phosphorylcholine (MPC, 97%, MW=295.27 g/mol, Sigma Aldrich), acetic acid (>99%, MW=60.05, Sigma Aldrich) and sodium chloride (NaCl, 99.5%, MW=58.44, Sigma Aldrich) were used as received. All solvents were of analytical grade purity and used without further treatment. Sulfabetaine methacrylate (ZB) was synthesized according to a previously published protocol (Sponchioni et al. Nanoscale, 2019, 11, 16582-16591). Rhodamine B-based methacrylate (HEMA-RhB) was synthesized via DCC-mediated esterification of HEMA and Rhodamine B (RhB) according to a previously published protocol (M. R. G. Kopp, et al., Ind. Eng. Chem. Res. 2018, 57, 7112). Briefly, 50 mg of RhB, 43 mg of DCC and 136 mg of HEMA were dissolved in 63 g of DCM and left to react overnight. The HEMA-RhB solution was then stored at 4° C.

Carboxybetaine methacrylate (CB) monomer was prepared as reported in literature (U. C. Palmiero, Biomacromolecules, 2018, 19, 1314-1323).

### Example 2: Copolymers Synthesis

The copolymers were synthesized via RAFT copolymerization using ACVA as initiator and CPA as RAFT agent by modifying previously published protocols (Sponchioni et al. Nanoscale, 2019, 11, 16582-16591; Sponchioni et al. J. Polym. Sci. Part A: Polym. Chem., 54: 2919-2931). In particular, all the sulfabetaine methacrylate (ZB)-based copolymers were synthesized in 20/80 vol/vol ethanol/3M NaCl acetic buffer (pH=4.5) at 10 wt. % total monomer concentration. The CPA to ACVA molar ratio was set constant to 3 while the monomer to CPA molar ratios (i.e. the degree of polymerization of the single monomer i (DPᵢ)) were varied according to Table S1 and Table S2:

TABLE S1
Complete characterization of the synthesized polymers

| | | | *DP_{HEMA +} _{Mam} | | Mn_{GPC} | | ƒ_{ZB} | X | Monomer |
|---|---|---|---|---|---|---|---|---|---|
| Sample | DP_{ZB} [—] | DP_{SB} [—] | [—] | DP_{Be} [—] | [g/mol] | Ð_{GPC} [—] | [mol. %] | [%] | distribution |
| ZW1 | 100 | 100 | — | — | 22048 | 1.1 | 47 | 98 | statistical |
| ZW2 | 130 | 70 | — | — | 21055 | 1.1 | 63 | 99 | statistical |
| ZW3 | 160 | 40 | — | — | 14544 | 1.2 | 76 | 99 | statistical |
| ZWH1 | 100 | — | 100 | — | 17752 | 1.1 | 54 | 95 | statistical |
| ZWH2 | 130 | — | 70 | — | 18942 | 1.2 | 69 | 97 | statistical |
| ZWH3 | 160 | — | 40 | — | 19577 | 1.1 | 81 | 98 | statistical |
| ZWBe | 100 | 70 | — | 30 | 13847 | 1.1 | 47 | 99 | statistical |
| EG | — | — | — | — | 6335 | 1.2 | — | 93 | — |

*DP_{HEMA}=DP_{Mam}

TABLE S2
Composition of other synthesized polymers

| | DP_{ZB} | DP_{SB} | DP_{MPC} | DP_{CB} | DP_{HSucc} | DP_{MQ} | DP_{DMAEMA} | DP_{MA} | DP_{SPMAK} | Monomer |
|---|---|---|---|---|---|---|---|---|---|---|
| Sample | [—] | [—] | [—] | [—] | [—] | [—] | [—] | [—] | [—] | distribution |
| ZW4 | 160 | 40 | — | — | — | — | — | — | — | statistical |
| | — | — | — | — | 5 | — | — | — | — | block |
| ZW5 | 200 | — | — | — | — | — | — | — | — | — |
| ZW6 | 400 | — | — | — | — | — | — | — | — | — |
| ZW7 | 800 | — | — | — | — | — | — | — | — | — |
| ZW8 | 190 | — | 10 | — | — | — | — | — | — | statistical |
| ZW9 | 180 | — | — | 20 | — | — | — | — | — | statistical |
| ZW10 | 150 | 50 | — | — | — | — | — | — | — | statistical |
| ZW11 | 190 | 10 | — | — | — | — | — | — | — | statistical |
| ZWN1 | 160 | — | — | — | — | — | — | 40 | — | statistical |
| ZWN2 | 150 | — | — | — | — | — | — | 50 | — | statistical |
| ZWN3 | 80 | — | — | — | — | — | — | 20 | — | statistical |
| ZWN4 | 170 | — | — | — | — | — | — | — | 30 | statistical |
| ZW + 1 | 160 | — | — | — | — | — | 40 | — | — | statistical |
| ZW + 2 | 190 | — | — | — | — | 10 | — | — | — | statistical |
| ZW + 3 | 180 | — | — | — | — | 20 | — | — | — | statistical |
| ZW + 4 | 170 | — | — | — | — | 30 | — | — | — | statistical |
| ZW + 5 | 160 | — | — | — | — | 40 | — | — | — | statistical |
| ZW + 6 | 320 | — | — | — | — | 80 | — | — | — | statistical |
| ZW + 7 | 480 | — | — | — | — | 120 | — | — | — | statistical |
| ZW + 8 | 80 | — | — | — | — | 20 | — | — | — | statistical |
| ZW + 9 | 80 | — | — | — | — | 20 | — | — | — | block |
| ZW + 10 | 160 | — | — | — | — | 40 | — | — | — | block |
| ZW + 11 | 160 | — | — | — | — | 60 | — | — | — | block |
| ZW + 12 | 480 | — | — | — | — | 120 | — | — | — | statistical |
| | — | — | — | 5 | — | — | — | — | — | block |
| ZW + 13 | 480 | — | — | — | — | 120 | — | — | — | statistical |
| | — | — | 50 | — | — | — | — | — | — | block |

As an example, in the case of ZW2 (i.e the copolymer with DP_{ZB}=130 and DP_{SB}=70 according to Table S1), 3.36 g of SB, 6.6 g of ZB, 48 mg of CPA, and 14 mg of ACVA were dissolved in 40 g of 20/80 vol/vol ethanol/1M NaCl acetic buffer (pH=4.5) and poured in a round bottom flask. The mixture was purged with nitrogen for 20 min and then heated to 65° C. for 24 h under constant stirring. The reaction mixture was dialyzed against 1 M NaCl for 3 days with a dialysis tubing (Spectra/Por®, molecular weight cut-off (MWCO)=3.5 kDa) by frequently changing the aqueous solution. The final polymer solutions were filtered with a 0.45 μm pore-size nylon membrane and stored at −20° C. Copolymer concentrations were evaluated via gravimetry. ZB-based copolymers covalently labeled with Rhodamine B were synthesized by adding a previously synthesized Rhodamine B-based methacrylate (HEMA-RhB) in the initial reaction mixture. The same amount of monomer weight of HEMA-RhB solution was poured in a round bottom flask and the solvent was removed under nitrogen before the addition of the other reagents. The same synthesis and purification protocol were then applied. However, the dialysis was performed for at least 1 week to remove all the unbound HEMA-RhB. In a similar way, a primary amine functional group was introduced in the SB-based copolymers by adding AEMA in the initial reaction mixture by setting DP_{AEMA}=1 and by following the same synthesis and purification protocols.

In the case of EG, 52 mg of CPA, 10 mg of ACVA, 1.05 g of EG₂MA and 268 mg of EGMA were dissolved in 5.5 g of ethanol and poured in a round bottom flask. The mixture was purged with nitrogen for 20 minutes and left to react at 65° C. for 2 days under constant stirring. The final copolymer was purified via precipitation in 45 mL of isopropyl ether. The recovered reddish waxy solid was dried under vacuum and stored at −20° C. EG covalently labeled with Rhodamine B was synthesized by adding HEMA-RhB in the initial reaction mixture. 1.27 g of this solution was poured in a round bottom flask and the solvent was removed under nitrogen before the addition of the other reagents. The same EG synthesis and purification protocols were then applied.

### Example 3: Gel Permeation Chromatography

The number-averaged molecular weight (Mn) and dispersity (D) of all the copolymers was evaluated via gel permeation chromatography. In the case of EG, a sample was dissolved at 4 mg mL⁻¹ in THF and filtered through a 0.45 μm pore-size PTFE membrane. The separation was performed on an Agilent set-up at a flow rate of 1 mL min⁻¹ at room temperature with a guard and two and two Agilent PLgel 20 μm MIXED-ALS columns. The values are reported in Table S1 are relative to poly(methyl methacrylate) standards. In the case of all the other copolymers, samples were dissolved at 4 mg mL⁻¹ in 0.05 M Na₂SO₄/acetonitrile (80/20 v/v) solution and filtered through a 0.45 am pore-size nylon membrane. The separation was performed on an Agilent set-up at a flow rate of 0.5 mL min⁻¹ at room temperature with a guard and two Suprema columns (particle size 10 mm and pore sizes 100 and 1000 Å, Polymer Standards Service). The values are reported in Table S1 and are relative to polyethylene glycol standards (ReadyCal-Kit PEO/PEG, Mp=238-969 000 Da, Polymer Standards Service).

### Example 4: Nuclear Magnetic Resonance

The conversion (X) and ZB molar fraction (f_{ZB}) of all the copolymers (Table S1) were evaluated via nuclear magnetic resonance (¹H-NMR) as reported in Sponchioni et al. Nanoscale, 2019, 11, 16582-16591. An aliquot of all the reaction mixtures were withdrawn before and after the reaction completion. The samples were dried under nitrogen, dissolved in either 3 M NaCl D₂O for all the SB-based copolymers or CDCl₂ for EG copolymers and analyzed on a Bruker NMR.

### Example 5: Cloud Point

Cloud points of the polymer droplets (Tcp, Table S3) were evaluated via dynamic light scattering (DLS) at 0.25 mg mL⁻¹ polymer concentration and at 150 mM NaCl. 200 μL of the sample were let to equilibrate at 65° C. for 20 minutes before analysis. The scattered light and droplet size was then measured from 65° C. to 5° C. with a temperature step of 1° C. and an equilibration time of 10 min. The cloud point was considered as the inflection point of the curve.

TABLE S3
Characterization of the polymer droplets

| | | | | | γ | | | |
|---|---|---|---|---|---|---|---|---|
| | T_{cp} | C_{m,salt} | W_{cont} | ρ | [mN | k_{RhB} | K_{BSA} | Polarity |
| Sample | [° C.] | [mM] | [wt. %] | [g ml⁻¹] | m⁻¹] | [—] | [—] | [—] |
| ZW1 | 7 ± 1 | 95 ± 5 | 48 ± 1 | 1.27 ± 0.07 | 77 ± 1 | 0.441 ± 0.001 | 0.11 ± 0.01 | 0.90 ± 0.06 |
| ZW2 | 24 ± 1 | 185 ± 5 | 53 ± 2 | 1.23 ± 0.05 | 81 ± 4 | 0.414 ± 0.004 | 0.13 ± 0.01 | 0.90 ± 0.06 |
| ZW3 | 41 ± 3 | 305 ± 5 | 45 ± 6 | 1.33 ± 0.10 | 78 ± 6 | 0.423 ± 0.004 | 0.07 ± 0.01 | 0.86 ± 0.03 |
| ZWH1 | 17 ± 2 | 125 ± 5 | 53 ± 4 | 1.16 ± 0.06 | 58 ± 2 | 3.172 ± 0.289 | 0.31 ± 0.01 | 0.89 ± 0.04 |
| ZWH2 | 31 ± 1 | 270 ± 5 | 53 ± 6 | 1.30 ± 0.01 | 74 ± 4 | 2.682 ± 0.062 | 0.18 ± 0.07 | 0.89 ± 0.01 |
| ZWH3 | 44 ± 2 | 395 ± 5 | 46 ± 3 | 1.31 ± 0.05 | 78 ± 1 | 1.956 ± 0.096 | 0.15 ± 0.02 | 0.91 ± 0.03 |
| ZWBe | 45 ± 1 | 325 ± 5 | 50 ± 3 | 1.23 ± 0.05 | 68 ± 1 | 4.563 ± 0.570 | 0.28 ± 0.01 | 0.66 ± 0.05 |
| EG | 19 ± 1 | — | 16 ± 2 | 1.21 ± 0.06 | 18 ± 9 | 18.99 ± 1.54 | 0.01 ± 0.03 | 0.47 ± 0.02 |

### Example 6: Critical Salt Concentration

The critical salt concentrations (C_{m,salt}, Table S3) at which the polymer droplets disappear were evaluated via wide-field microscopy using a dichotomous search method. 0.25 mg mL⁻¹ of the polymer aqueous solutions at different NaCl concentrations were incubated at room temperature in a 384-well plate (MatriPLate, Glass Bottom, Brooks) and imaged after 24 hours.

### Example 7: Physical Characterization of the Polymer Dispersed Phase

Pure polymer-rich phases were obtained via dialysis of polymer solutions against distilled water at room temperature for 2 days with a membrane tubing (MWCO=3.5 kDa, SpectrPor®). The dispersed phases were collected at the bottom of the membrane tubing and stored wet at 4° C. The water content (W_{cont} in Table S3) and density (p in Table S3) of the polymer dispersed phases were evaluated via gravimetry of known volume samples. The interfacial tension (yin Table S3) was evaluated with a contact angle system OCA 15 plus. The polymer phase was loaded in a 1 mL syringe loaded with a 25G gauge needle with a flat tip. The interfacial tension was evaluated using the SCA20 software (dataphysics).

### Example 8: Droplet Polarity

The polarity of the droplets (Polarity in Table S3) was measured using PRODAN as solvatochromic dye according to a previously published protocol (A. M. KOffner et al. ChemSystemsChem 2020, 2, e2000001). Briefly, the emission spectrum of PRODAN was acquired in different droplets between 400 and 600 nm with a 5 nm wide spectral window using a fluorescence confocal microscope. The wavelength that corresponds to the maximum fluorescent emission was used to evaluate the relative polarity of the droplets by applying a linear fit of reference solvents with different polarities (see C. C. Vequi-Suplicyet al. J. Fluoresc. 2015, 25, 621-629; O. A. Kucherak et al. J. Phys. Chem. Lett. 2010, 1, 616-620).

### Example 9: Droplet Liquidity

The liquidity of the droplet was evaluated via fluorescence recovery after photo-bleaching (FRAP) according to a previously published protocol (Nicole O. Taylor et al., Biophysical Journal, 117, 7, 2019, 1285-1300). 5 mg mL¹ of RhB covalently labeled-polymer in 90 mM NaCl aqueous solutions were incubated at room temperature for 24 hours and then photo-bleached at 561 nm. The photo-bleached spot radius was set equal to 1/10 of the droplet radius. Result are an average of at least three different droplets. Droplet fusion events were also monitored on chip.

### Example 10: Protein Synthesis and Characterization

DDX4 (LCD) was produced in E. coli BL21-GOLD (DE3) cells. Protein production was induced at optical density (OD) around 0.7 with 0.5 mM isopropyl d-thiogalactopyranoside (99%, PanReac AppliChem). After 16 h at 37° C., the recombinant proteins were purified using His-tag immobilized metal ion affinity chromatography (Chelating Sepharose, GE Healtchare). The proteins were further purified via size exclusion chromatography using a Superdex 75 26/600 on an AKTA Prime system (GE Healthcare) in 50 mM Tris at pH=7.5 and 500 mM NaCl. The quality of the purified proteins was confirmed by SDS-PAGE electrophoresis. The proteins were concentrated to 600-700 μM and aliquots were frozen and stored at −20° C.

TABLE S4
Partitioning coefficient of different molecules and macromolecules in droplets
constituted by different polymers

| | EG | ZW2 | ZWH1 | ZWH+ | ZWBe | LCD |
|---|---|---|---|---|---|---|
| Rhodamine | 18.99 ± 1.54 | 0.414 ± 0.004 | 3.172 ± 0.289 | 1.72 ± 0.04 | 4.563 ± 0.570 | 22.22 ± 1.6 |
| B | | | | | | |
| Fluorescein | 0.31 ± 0.1 | 0.305 ± 0.05 | 1.97 ± 0.04 | 2.44 ± 0.24 | | 3.66 ± 0.02 |
| BSA | 0.01 ± 0.03 | 0.13 ± 0.1 | 0.31 ± 0.01 | 3.05 ± 1.4 | 0.28 ± 0.01 | 27 ± 5 |
| IgG | 0.39 ± 0.15 | 0.19 ± 0.01 | 0.35 ± 0.17 | 0.4 ± 0.02 | 0.14 ± 0.02 | 1.54 ± 0.12 |
| Dextran | 0 ± 0.01 | 0.08 ± 0.02 | 0.04 ± 0.05 | 0.2 ± 0.03 | 0.01 ± 0.01 | 1.62 ± 0.01 |
| 4 kDa | | | | | | |
| Dextran | 0.48 ± 0.01 | 0.33 ± 0.08 | 0.34 ± 0.13 | 0.58 ± 0.01 | 0.40 ± 0.14 | 0.67 ± 0.01 |
| 150 kDa | | | | | | |

### Example 11: Evaluation of Solute Partition Coefficients

The partition coefficient of the solutes between different phases was evaluated via confocal fluorescence microscopy as K=(I_{droplet}−I_{background})/(I_{bulk}−I_{background}) where I _{droplet} and I _{bulk} are the average fluorescence intensity inside and outside the droplets, respectively, and I_{background} is the average fluorescence intensity of the samples at the same conditions, but without the presence of any fluorescent solute. The values reported are an average of at least three different micrographs.

### Example 12: Polymer Conjugation

23.4 mg of ZW10, 7.2 mg NHS and 4.8 mg EDC were dissolved in 500 μL MES buffer (0.1 M MES, 1 M NaCl, pH 6.0) and reacted at room temperature for 15 min. The pH was raised to 7.2 with 1 M Na2CO3 and a solution of either 2.4 mg biotin-PEG7-NH2 or 4 mg Protein A in 50 μL coupling buffer (0.1 M Na-phosphate, 1 M NaCl, pH 7.2) was quickly added and gently shaken at room temperature for 1 hour. Then, ethanolamine was added to the solution to achieve a final concentration of 1 M and shaken at room temperature for 30 min. The solutions were diluted 30 times in Millipore water and kept at 4° C. for at least 2 hours to promote phase separation. The solution was centrifuged at 6000×rcf for 10 minutes. The supernatant was removed and the solution was resuspended in 500 μL 5×PBS or 5× Protein A equilibration buffer (0.1 M Na-phosphate, 0.75 M NaCl, pH 7.5), respectively. The solutions were then diluted, incubated and centrifuged as before. The separated polymer conjugates were then resuspended to achieve theoretical final polymer concentrations of 200 mg/mL. Similarly, 23.4 mg ZW11 were activated with NHS esters. Then, 15 μL of the polymer solution at pH 7.2 were added to 100 μg streptavidin in 10 mM phosphate (10 μL) and 2.5 μL high salt buffer (5 M NaCl, 2.7 mM KCl, 10 mM Na2HPO4, 1.8 mM KH2PO4, pH 8.8) and shaken at room temperature for 1 hour. The solution was then mixed, shaken with ethanolamine and washed as previously described. 5×PBS with 1 M NaCl was used as resuspension buffer.

### Example 13: Microscope Analysis of Bioseparation

Biotin-streptavidin: 1 μg streptavidin-ATT0425 was premixed with 37 μg BSA, 74 μL PBS and 20 μL Millipore water. Then, approximately 25 μg (5 μL) of either ZW10-biotin or ZW10 (control) in 5×PBS was added to the well and let phase separate at room temperature. Protein A-IgG: 6 ag IgG-ATTO647N was premixed with 80.5 μL Protein A equilibration buffer and 14 μL Millipore water. Then, approximately 87 ag (3.5 μL) of either ZW10-Protein A or ZW10 (control) in 5×Protein A equilibration buffer was added to the well and let phase separate at room temperature. Streptavidin-biotinylated liposomes: biotinylated-rhodamine B liposomes were premixed with 47 μL PBS and 45 μL Millipore water. Then, approximately 25 ag (3.5 μL) of either ZW11-streptavidin or ZW11 (control) in 5×PBS with 1 M NaCl was added to the well and let phase separate at room temperature.

### Example 14: Purity of Bioseparated Species

A protein solution was obtained spiking 1200 μL Millipore water and 500 μL Protein A equilibration buffer (20 mM Na-phosphate, 0.15 M NaCl, pH 7.5) with 100 μg bovine serum albumin (BSA), 100 μg adenylate kinase (AK), 100 μg insulin, 80 μg Immunoglobulin G (IgG) and, when specified, with 500 μL cell lysate. 80 μL of a 134 mg/mL ZW11 or Protein A-conjugated ZW11 solution were added to 735 μL of the protein solution, quickly vortexed and incubated at room temperature for 15 minutes and at 4° C. for 15 minutes. The samples were centrifuged at 3000 rcf and 4° C. for 1 minutes and the supernatants were removed. The polymer phases were first dissolved in 100 μL 7×protein A equilibration buffer (0.14 M Na-phosphate, 1.05 M NaCl, pH 7.5), then diluted in 600 μL Millipore water and incubated at 4° C. for 15 minutes. The samples were centrifuged as before and the supernatants removed. The polymer phase was dissolved in 150 μL high salt elution buffer (0.1 M citric acid, 0.5 M NaCl, pH 3.0), diluted with 300 μL low salt elution buffer (0.1 M citric acid, pH 3.0) and incubated at 4° C. for 15 minutes and centrifuged as before. The elution supernatants were then collected and analyzed by size exclusion chromatography after increasing their pH to 6.3 with 1 M Tris buffer at pH 9.0.

### Example 15: Microfluidics

To monitor the size distributions over time as well as the UCST and LCST behavior of the ZW2 and the EG polymer, the droplets were encapsulated into water-in-oil emulsions using a microfluidic device with a flow focusing geometry. The microfluidic devices were fabricated as previously described (ML, Angew. Chem. 58 (41), 2019, 14498-14494). In brief, a master wafer was fabricated by spin-coating SU-8 photoresist polymer (MicroChem) on a silicon wafer. Afterwards, a mask was applied on top of the polymer layer, containing the desired pattern of the channels. After UV polymerization, hardening and developing, the desired channel structure was left as relief on the master wafer. The chips were realized by standard soft lithography: polydimethylsiloxane (PDMS) was mixed with curing agent (Sylgard 184, Silicone Elastomer Base/Curing Agent, Dow Corning) at a ratio of 10:1, poured on the master wafer, degassed and cured for 30 min at 70° C. The same procedure was carried out with a blank master wafer without channel structures to fabricate a thin PDMS bottom layer (about 2 mm). To obtain functional channels, the two layers were laid on top of each other and inter-connected by further crosslinking over night at around 110° C. In case of the ZW2, the phase transition was induced on chip. The chip contains three inlets and one outlet as well as two junctions where fluids can be mixed in a highly controllable manner. At the first junction, the homogenous polymer solution (10 mg/ml stock in 500 mM NaCl) was mixed with H2O with a flow rate ratio of 2:1 (0.4 μl/min and 0.2 μl/min, respectively are absolute flow rates) using syringe pumps (Cetoni, neMESYS, Cetoni GmbH). This induced a rapid drop in the salt concentration, resulting in the formation of liquid-like, phase separated polymer droplets at 3.3 mg/ml polymer in 166.67 mM NaCl. These droplets were then encapsulated into water-in-oil compartments at a second junction, by mixing them with HFE-7500 oil (Acota Limited) supplied with 0.2% surfactant (Pico-Surf 1, Sphere Fluidics, Cambridge, UK). After formation, the compartments containing the phase-separated droplets were transferred into capillaries (0.1 mm inner diameter; CM Scientific) and observed off chip. Droplet coalescence was observed over a time scale of 8 min until only a single droplet was left in each compartment. To heat the encapsulated phase-separated droplets, the capillaries were transferred to a glass slide, covered with a thermo-conductive coating and connected to a power supply (RND-320 KD3005D, Distrelec). Applying voltage ranging from 0 to 3 V increased the temperature from around 25° C. to around 40° C., which was measured by a thermocouple (NI 9211, National Instruments) connected to the NI MAX LABView software. While heating/cooling the sample, the disappearance and re-appearance of the droplets was monitored over time. The encapsulation and heating of the RhB-labeled polymers were observed on a Nikon Eclipse Ti-E epifluorescence microscope. The RhB-labeled polymers were excited (λexc=546 nm) with a LEDHub light source (Omicron LedHUB Light engine) and detected (λexc=568 nm) using an Andor Zyla sCMOS camera. For the EG polymer, the phase separation was induced off chip by equilibrating a solution of 1.25 mg/ml polymer at room temperature. The resulting droplets were encapsulated into water-in-oil compartments and subsequently transferred into capillaries. To cool the samples, an in-house built cooling stage was used to decrease the temperature of the encapsulated droplets to 14.5° C., below the Tcp of the polymer. Images were taken as described above. Image analysis was carried out by using a Matlab code to extract droplets radii over time.

### Example 16: In-Situ Analysis of a Biomarker Via Microfluidics

In bulk, the ZW-droplet assay has a theoretical upconcentration limit of 10×, considering an initial sample of 200 μL and a final ZW condensed phase enriched in the target analyte of 20 μL. In order to achieve a much higher concentration factor, we implemented the assay in a microfluidic format. In this case, the shrinkage of the compartments is replaced by the formation of a polymer-rich droplet within the compartments. Assuming a microcompartment size of 100 μm diameter and a polymer-rich droplet upconcentrating the analyte of less than 5 μm, we could achieve a concentration factor of approximately 10′000×. As model analytes ZW10 conjugated to Biotin were used to recruit fluorescently labeled streptavidin (ATTO 425-Streptavidin, 09260, Sigma Aldrich). The microfluidic device used for droplet generation consisted of two polydimethylsiloxane (PDMS, Silicone elastomer 184, Sylgard 184 kit, Dow Corning, USA) layers bonded to each other. The first layer was thick (approximately 10 mm) and contained the droplet generation module with a nozzle measuring 50 μm width and a channel with a 1600 μm wide and 9500 am long geometry. The mold corresponding to this layer was prepared by spin-coating SU-8 photoresist on silica wafers (MicroChemicals, Germany). Selected regions of the coated wafer were exposed to UV light for curing. The second layer consisted of a flat and thin (<1 mm) PDMS layer. Standard soft-lithography was used to replicate the channels and to cast the flat PDMS layer on a flat wafer. PDMS mixed with the corresponding cross-linker at a 15:1 weight ratio was cast on the molds and degassed under vacuum at 10 mbar for 30 min. Partial curing of these PDMS layers was achieved by incubation for 25 min at 70° C. After peeling off the PDMS layers from the wafers, the channel layer was aligned and contact bonded to the flat layer. Full bonding of the two layers and complete curing of the PDMS were achieved by incubating the device at 60° C. overnight. Water-in-oil droplets were generated in this device by emulsifying the analyte and polymer droplet containing solutions in Fluoridrop 7500 oil (Dolomite Microfluidics, United Kingdom) with 0.1% Picosurf 1 (Dolomite Microfluidics) as surfactant. The flow in the channels was imposed with syringe pumps (Cetoni neMESYS, Cetoni GmbH, Germany). The droplets generated on the device were collected in square quartz capillaries (0.1 mm inner diameter, CM Scientific, UK) and incubated for 30 min to allow analyte recruitment and the merging of all ZW droplets into a single LLPS phase. The droplets were then imaged on Ti2-U epi-fluorescence inverted microscope (Nikon, Switzerland) equipped with an automatic stage, LED light sources operating at 455 nm and 617 nm (Omicron Laserage Laserprodukte GmbH, Germany), and a camera (Zyla sCMOS 4.2P-CL10, Andor, United Kingdom). The different analytes were detected with filter cubes sets (AHF Analysentechnik AG, Germany) at the following excitation/emission wavelengths: CFT ET filter set (436 nm-480 nm) for the labeled streptavidin, and a Cy5 ET Filter set filter set (640 nm/690 nm) for the ATTO647N tagged detection antibodies. Droplets were imaged with a 20× magnification objective (CFI Plan Apo Lambda 20X, Nikon, Switzerland). Bulk samples were prepared in 384-well plates (MatriPlate 384 Well Plate, Glass Bottom, Brooks). The recruitment of labeled streptavidin was tested by ZW11-Biotin, and Aβ42 by ZW4-HET, where HET corresponds to an Aβ42 antibody binding fragment.

### Example 17: Microscope Analysis of Bioseparation

Charged droplets-liposomes: each well was prepared to obtain 100 μL mixtures of liposomes and droplets at 100 mM NaCl and 20 mM Na-phosphates. First, DOPS-liposomes labelled with rhodamine-B were diluted with Na-phosphate buffer to either 8*10¹⁰ or 4*10⁹ particles/mL. Then, solutions of ZW+8 or ZW+9 were added to achieve a final polymer concentration of 0.15 mg/mL. The mixtures were incubated at room temperature for 2 hours and imaged with an inverted fluorescence microscope.

### Example 18: Separation of Extracellular Vesicles (EVs)

HEK293-F cells (Gibco) were cultured at 37° C., pH 7.1, DO 40% and 250 rpm in CD 293 medium (Gibco) supplemented with 4×10⁻³ M GlutaMAX and 250 mg L⁻¹ Pluronic F-68 for 166 h. Conditioned media (500 mL) was harvested from ≈1×10⁹ cells with 93% viability by centrifugation at 200×g for 5 minutes and at 3000×g for 15 min and stored at −20° C. Conditioned media (50 mL) was then clarified by 0.22-μm filtration and concentrated 100 times using an Amicon Ultra-15 centrifugal filter (RC membrane, 50 kDa MWCO, Merck Millipore). EVs were isolated by gravity flow size exclusion chromatography in a 10 mL column packed with CL4B resin (Cytiva) and fractions of 500 μL were collected. Fractions 6-8 were pooled, aliquoted and stored at −80° C. The EVs were then used to analyze their recruitment and release in ZW-based droplets. Mixtures (300 μL) of EVs and ZW+8 at 25 mM NaCl and 20 mM Na-phosphate were prepared. First, EVs were diluted with Na-phosphate buffer to 3.3*10⁹ particles/mL. Then, ZW+8 dissolved in a NaCl solution was added to the EV dilutions to achieve a final polymer concentration of 0.25 mg/mL. The mixtures were incubated at either 4° C. or 25° C. for either 1 or 15 minutes. After incubation, the mixtures were centrifuged at 6000×g and 4° C. for 5 minutes to pellet the droplets. The supernatants (270 μL) were removed and 270 μL of buffer (20 mM Na-phosphate, 608 mM NaCl, pH 7.4) were added to screen the interactions between the polymer and the EVs and resuspend the pellets. The eluates were centrifuged at 3000×g and 25° C. for 15 minutes to remove aggregates. All supernatant and eluates were then analyzed by ELISA. The mixtures (50 μL) were diluted with Na-phosphate buffer (50 μL) to get 800 mM NaCl and 20 mM Na-phosphate per well, incubated overnight, blocked with 1% BSA and stained with anti-CD81 antibody. Dynamic light scattering was used to analyze the size distribution of the initial EVs and of the EVs eluted from the droplets incubated with EVs for 1 minute at 25° C. EVs eluted from droplets were also analyzed by TEM to confirm their integrity. A mixture (600 μL) of EVs and ZW+8 at 25 mM NaCl and 20 mM Na-phosphates was prepared as described above. The droplets were incubated with the sample for 1 minute at 25° C. and isolated as described above. EVs in the eluate were diafiltered in a VivaSpin 500 centrifugal filter (300 kDa MWCO, PES membrane, Sartorius) first with a high salt buffer (550 mM NaCl, 20 mM Na-phosphate, pH 7.4) to remove the polymer and then with a low salt buffer (150 mM NaCl, 20 mM Na-phosphate, pH 7.4) to decrease the salt concentration. The eluate was finally concentrated to 10 μL, adsorbed on carbon-coated grids, stained with 2% uranyl acetate and imaged with a TEM JEOL JEM 1400 microscope.

### Example 19: Uptake of Species in Charged Droplets

Either rhodamine-labelled DOPS liposomes, pure EVs or clarified conditioned medium (CCM) produced as described in Example 18 were used to study the uptake of liposomes, EVs or other impurities (e.g. DNA and proteins) in the droplets. Liposomes were diluted to get mixtures at pH 7.4 with 10⁹ particles/mL, 20 mM Na-phosphate, NaCl concentrations between 25 and 400 mM and either 0.25 mg/mL ZW+8 or 0.25 mg/mL ZW+9. EVs were diluted to achieve final mixtures at pH 7.4 containing 3.3*10⁹ particles/mL, 20 mM Na-phosphate, NaCl concentrations between 25 and 400 mM and 0.25 mg/mL ZW+8. CCM was diluted 20 times with buffers to get mixtures with 20 mM Na-phosphate, NaCl concentrations between 25 and 400 mM and 0.25 mg/mL ZW+8. The samples were incubated for 15 minutes at room temperature and centrifuged for 15 minutes at 3000×g and 25° C. The supernatants were then collected to analyze the amounts of liposomes in solution by fluorescence, CD81-positive EVs by ELISA, of DNA with a QuantIT dsDNA assay kit (ThermoFisher) and of proteins with a Micro BCA protein assay kit (ThermoFisher).

### Example 20: Droplet Interaction with Surfaces

Block copolymers ZW+12 and ZW+13 were synthetized as described in Example 2. HEMA-Rhodamine was added to make the polymers fluorescent. Droplets were formed diluting the polymers to 0.03 mg/mL in 50 mM NaCl and their interaction with glass was analyzed by fluorescence microscopy.

### Example 21—Uptake of Monoclonal Antibodies

99±1% uptake of an IgG1 antibody was achieved with polymers ZW2, ZW5, ZW6 and ZW7 coupled to Protein A. Recombinant Protein A was covalently coupled to the carboxyl group resulting from the RAFT polymerization initiator either through the primary amine groups of the protein with EDC/NHS reaction or through the terminal thiol group of the recombinant Protein A with EDC/NHS reaction and a N-(2-Aminoethylmaleimide) linker. The antibody was incubated with the droplets in phosphate buffer (20 mM Na-phosphate, 150 mM NaCl, pH 7.5). Uptake of an IgG1-ATTO647N antibody was also observed by fluorescence microscopy in droplets of ZW8 and ZW9 coupled to Protein A as reported in this example and in the droplets composed of ZWN1, ZWN2, ZWN3 and ZWN4. The antibody was incubated with ZW8 and ZW9 droplets in phosphate buffer (20 mM Na-phosphate, 150 mM NaCl, pH 7.5) and with the ZWN1, ZWN2, ZWN3 and ZWN4 droplets in citrate phosphate buffer (18 mM citrate, 13 mM Na-phosphate, pH 4.5).

### Example 22—Uptake of Albumin

Up to 40-95% uptake of bovine serum albumin tagged with FITC was achieved with copolymers ZW+2, ZW+3, ZW+4, ZW+5, ZW+6, ZW+7 and ZW+8. Albumin was incubated with the droplets in phosphate buffer (20 mM Na-phosphate, 37.5 mM NaCl, pH 7.4). Uptake of BSA-FITC was also observed by fluorescence microscopy in ZW+1 when BSA-FITC was incubated with the droplets in phosphate buffer (20 mM Na-phosphate, 37.5 mM NaCl, pH 6.5).

### Example 23—Uptake of Liposomes and Extracellular Vesicles

99±1% uptake of liposomes made of 1,2-dioleoyl-sn-glycero-3-phospho-L-serine and of extracellular vesicles was achieved with ZW+7 and ZW+8. Liposomes were incubated with the droplets in phosphate buffer with 100 mM NaCl (20 mM Na-phosphate, 100 mM NaCl, pH 7.4). Extracellular vesicles were incubated with the droplets in a phosphate buffer with 25 mM NaCl (20 mM Na-phosphate, 25 mM NaCl, pH 7.4). 99±1% uptake of the same liposomes and of extracellular vesicles was also achieved with block copolymers ZW+9, ZW+10 and ZW+11. Liposomes were incubated with the droplets of ZW+9 in phosphate buffer with NaCl concentrations between 100 and 200 mM (20 mM Na-phosphate, 100-200 mM NaCl, pH 7.4), while with droplets of ZW+10 and ZW+11 in phosphate buffer with NaCl concentrations between 100 and 250 mM (20 mM Na-phosphate, 100-250 mM NaCl, pH 7.4). Extracellular vesicles were incubated with the droplets in the same phosphate buffer, but at NaCl concentrations between 25 and 37.5 mM with ZW+9, between 25 and 50 mM with ZW+10 and between 25 and 75 mM with ZW+11.

1.-15. (canceled)

16. A method for taking up compounds from outside of liquid droplets into the droplets in an aqueous medium, wherein the method comprises the steps of:
(i) providing a polymer according to Formula (I) 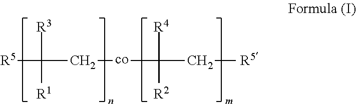
wherein:
n is an integer selected from 0 to 2500;
m is an integer selected from 2 to 2500;
R³ and R⁴ are each independently selected from the group consisting of hydrogen and methyl;
R⁵ and R^{5′} are independently selected from the group consisting of
hydrogen, linear or branched (C₁₋₁₀)alkyl, a halide, or phenyl;
a group resulting from a polymerization initiator; and
a target binding moiety (TBM);
R¹ is selected from the group consisting of: 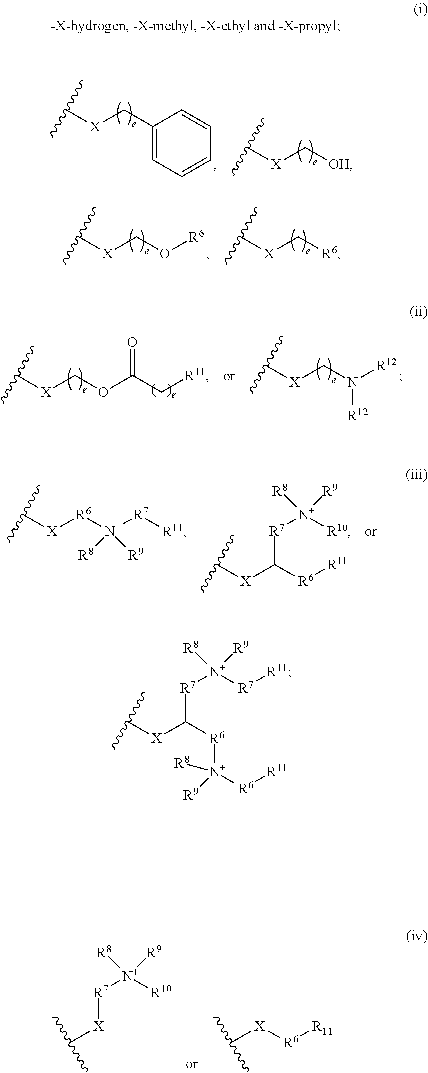
or in a ratio of about 1:1 within the polymer; 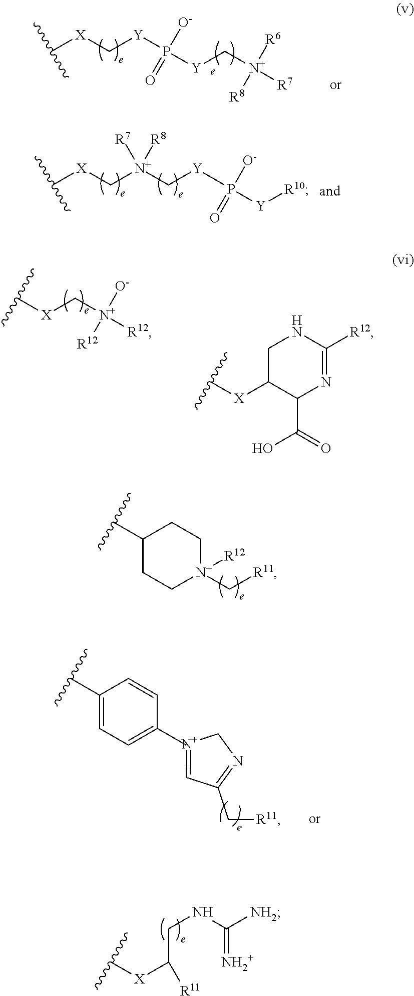
R² is selected from the group consisting of 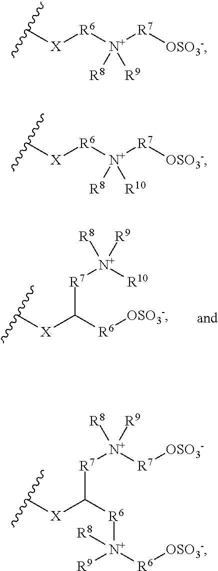
wherein:
X is selected from the group consisting of —C(═O)—O—, —C(═O)—NH—, aliphatic carbocycle, aromatic carbocycle, aliphatic heterocycle, and aromatic heterocycle;
Y is oxygen or absent;
e is an integer selected from 1 to 5;
R⁶ and R⁷ are each independently selected from the group consisting of:
linear or branched, substituted or non-substituted (C₁₋₁₀)alkyl,
linear or branched, substituted or non-substituted (C₁₋₁₀)alkyl comprising one
or more heteroatoms selected from the group consisting of O, N, S and P, (C₂₋₁₀)alkenyl, and
phenyl, and an aromatic heterocycle;
R⁸, R⁹ and R¹⁰ are each independently selected from the group consisting of hydrogen, linear or branched, substituted or non-substituted (C₁₋₁₀)alkyl, (C₂₋₁₀)alkenyl, phenyl, an aromatic heterocycle, 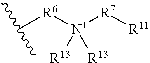
when forming R¹, and 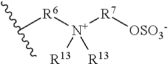
when forming R²;
R¹¹ is selected from the group consisting of —SO₃⁻, —C(═O)O⁻, and ON; 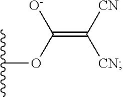 [SMILES: *OC([O-])=C(C#N)C#N]
R¹² is selected from the group consisting of methyl, ethyl, and propyl;
R¹³ is independently selected from the group consisting of linear or branched, substituted or non-substituted (C₁₋₁₀)alkyl, (C₂₋₁₀)alkenyl, phenyl, and an aromatic heterocycle;
(ii) forming liquid droplets from the polymer of step (i) in an aqueous medium; and
(iii) taking up compounds from outside of the liquid droplets into the droplets in the aqueous medium for separation, storage, and/or reaction of the compounds inside the liquid droplets.

17. The method according to claim 16, wherein:
n is an integer selected from 10 to 250;
m is an integer selected from 10 to 250;
e is an integer selected from 2 to 3;
X is selected from the group consisting of —C(═O)—O—, —C(═O)—NH—, pyridine, piperidine, and phenyl;
R⁶ is (C₁₋₅)alkyl;
R⁷ is (C₁₋₅)alkyl;
R⁸, R⁹ and R¹⁰ are each independently selected from the group consisting of methyl, 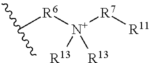
when forming R¹, and 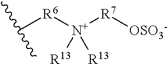
when forming R²;
R¹¹ is —SO₃⁻ or —C(═O)O⁻;
R¹² is methyl; and
R¹³ is selected from the group consisting of methyl, ethyl, and propyl.

18. The method according to claim 16, wherein
R¹ is selected from the group consisting of 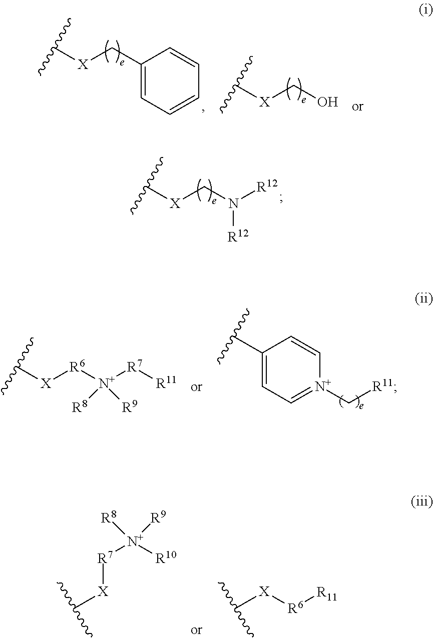
in a ratio of about 1:1 within the polymer; 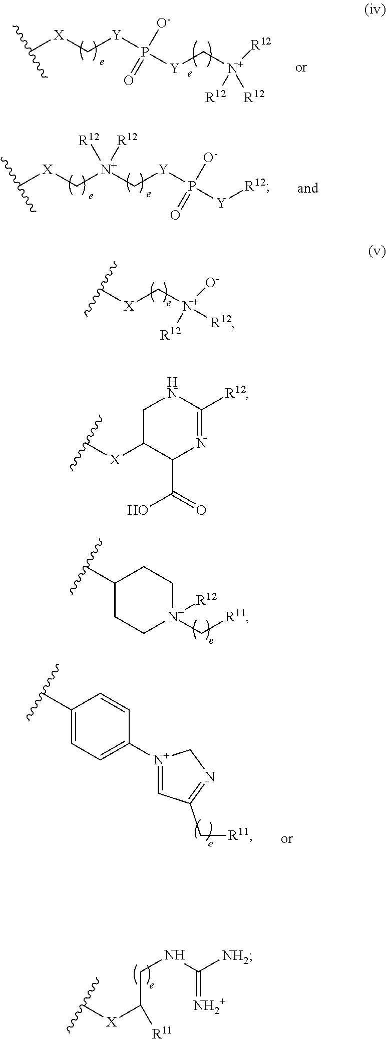
and
R² is

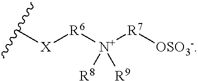

19. The method according to claim 16, wherein
n is an integer selected from 10 to 250;
m is an integer selected from 10 to 250;
R⁵ and R^{5′} are hydrogen or independently selected from the group consisting of azide, alkyne, carboxylic acid, amine, (C₂₋₁₀)alkenyl, streptavidin, avidin, biotin, an aptamer, a peptide, a protein, an oligonucleotide, a cell, an antibody, a molecularly imprinted polymer, an affinity polymer, a positively or negatively charged oligopolymer and an organic or inorganic nanoparticle;
R¹ is selected from the group consisting of 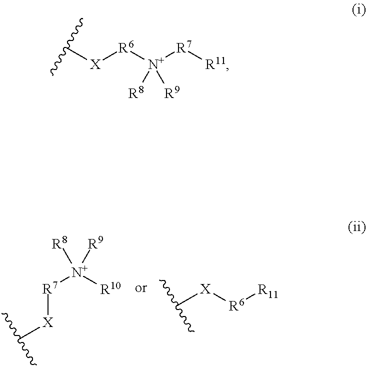
in a ratio of about 1:1 within the polymer; 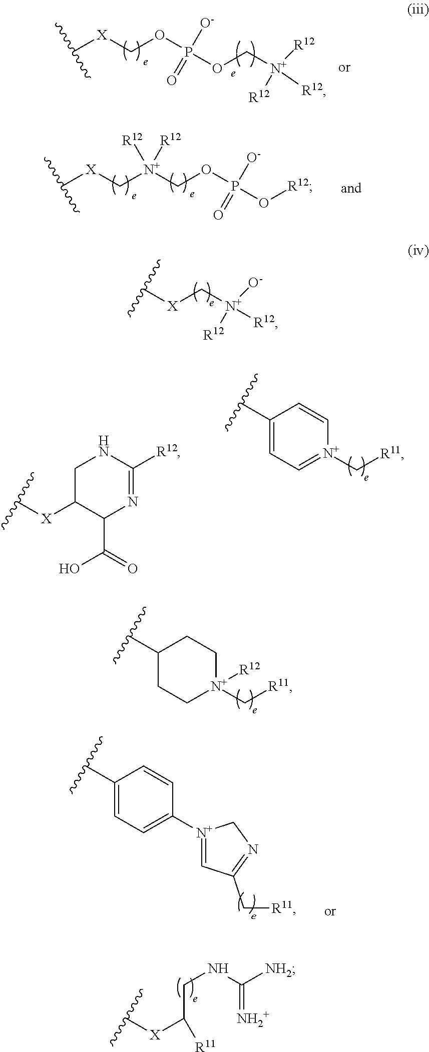
R² is 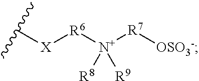
wherein
R⁶ and R⁷ are each independently selected from the group consisting of methyl, ethyl, and propyl;
R⁸, R⁹, R¹⁰, R¹², and R¹³ are each methyl; and
R¹¹ is —SO₃⁻ or —C(═O)O⁻.

20. The method according to claim 16, wherein
R¹ is selected from the group consisting of 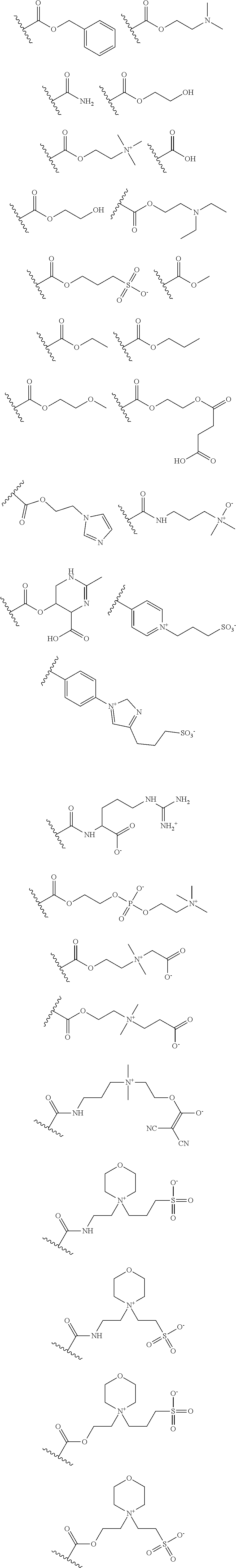 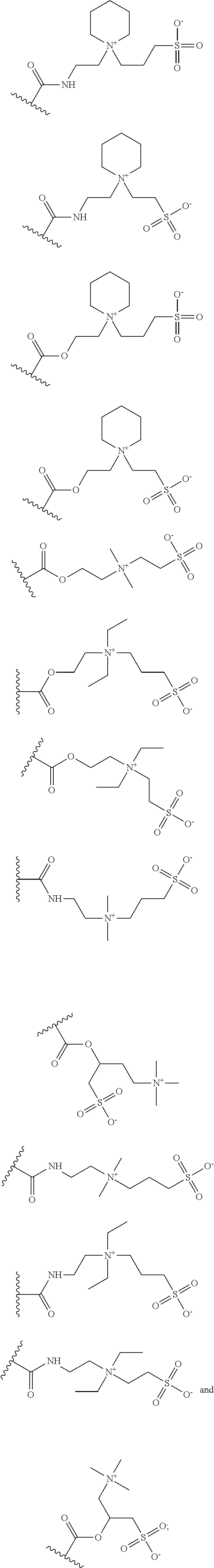
and
R² is selected from the group consisting of

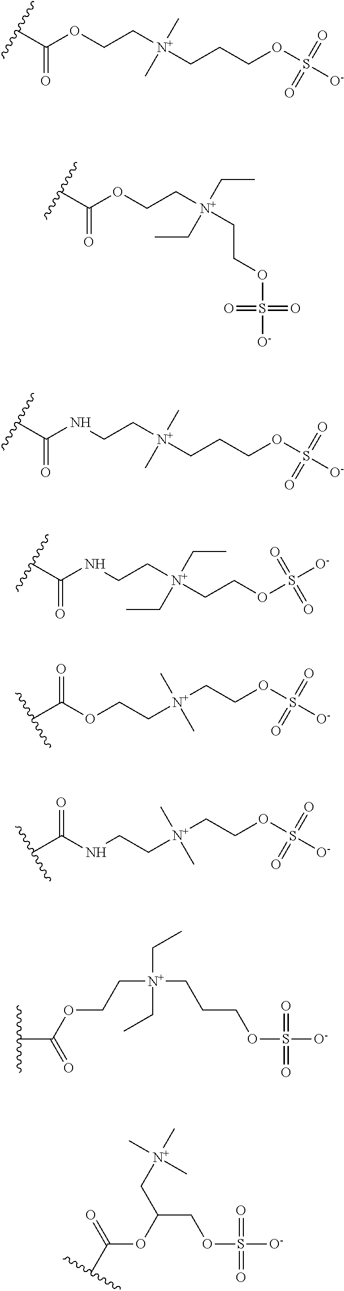

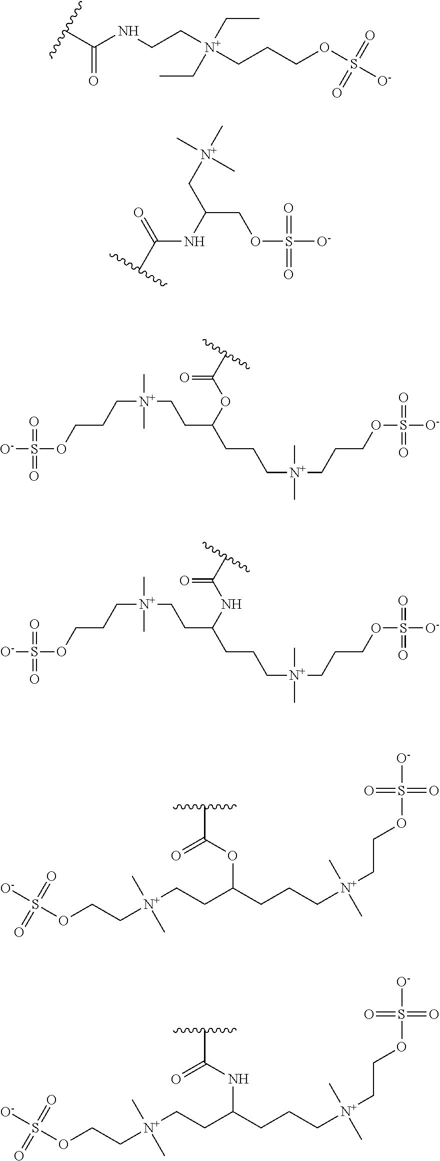 [SMILES: *C(=O)NCC[N+](CC)(CC)CCCOS(=O)(=O)[O-] ; *C(=O)NC(COS(=O)(=O)[O-])C[N+](C)(C)C ; *C(=O)OC(CCC[N+](C)(C)CCCOS(=O)(=O)[O-])CC[N+](C)(C)CCCOS(=O)(=O)[O-] ; *C(=O)NC(CCC[N+](C)(C)CCCOS(=O)(=O)[O-])CC[N+](C)(C)CCCOS(=O)(=O)[O-] ; *C(=O)OC(CCC[N+](C)(C)CCOS(=O)(=O)[O-])CC[N+](C)(C)CCOS(=O)(=O)[O-] ; *C(=O)NC(CCC[N+](C)(C)CCOS(=O)(=O)[O-])CC[N+](C)(C)CCOS(=O)(=O)[O-]]

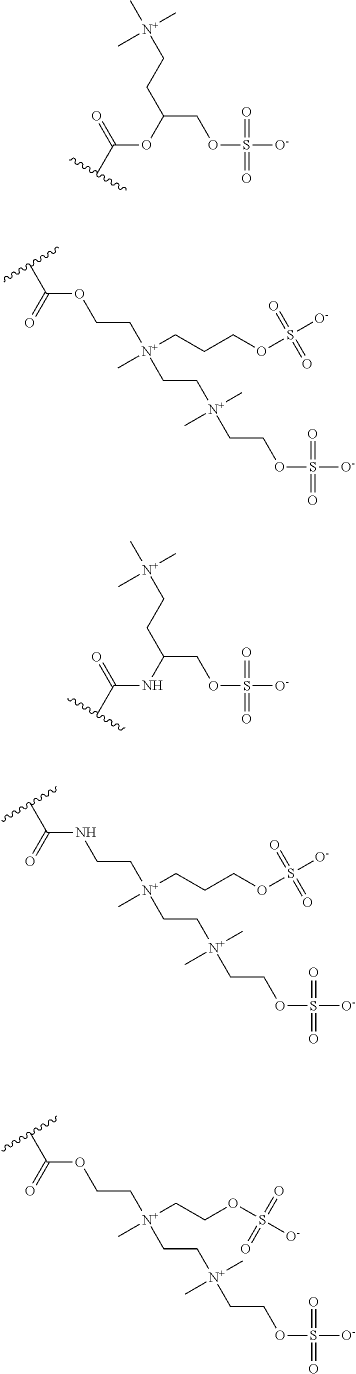 [SMILES: *C(=O)OC(CC[N+](C)(C)C)COS(=O)(=O)[O-] ; *C(=O)OCC[N+](C)(CCCOS(=O)(=O)[O-])CC[N+](C)(C)CCOS(=O)(=O)[O-] ; *C(=O)NC(CC[N+](C)(C)C)COS(=O)(=O)[O-] ; *C(=O)NCC[N+](C)(CCCOS(=O)(=O)[O-])CC[N+](C)(C)CCOS(=O)(=O)[O-] ; *C(=O)OCC[N+](C)(CCOS(=O)(=O)[O-])CC[N+](C)(C)CCOS(=O)(=O)[O-]]

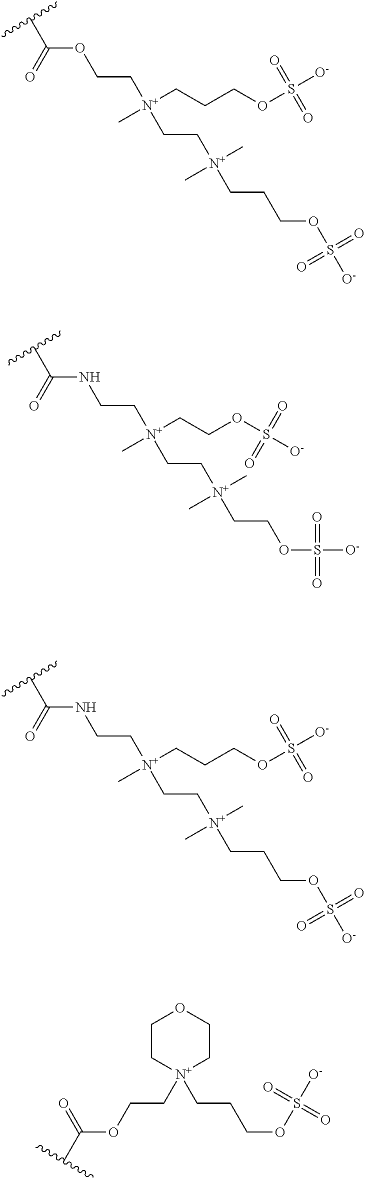 [SMILES: *C(=O)OCC[N+](C)(CCCOS(=O)(=O)[O-])CC[N+](C)(C)CCCOS(=O)(=O)[O-] ; *C(=O)NCC[N+](C)(CCOS(=O)(=O)[O-])CC[N+](C)(C)CCOS(=O)(=O)[O-] ; *C(=O)NCC[N+](C)(CCCOS(=O)(=O)[O-])CC[N+](C)(C)CCCOS(=O)(=O)[O-] ; *C(=O)OCC[N+]1(CCCOS(=O)(=O)[O-])CCOCC1]

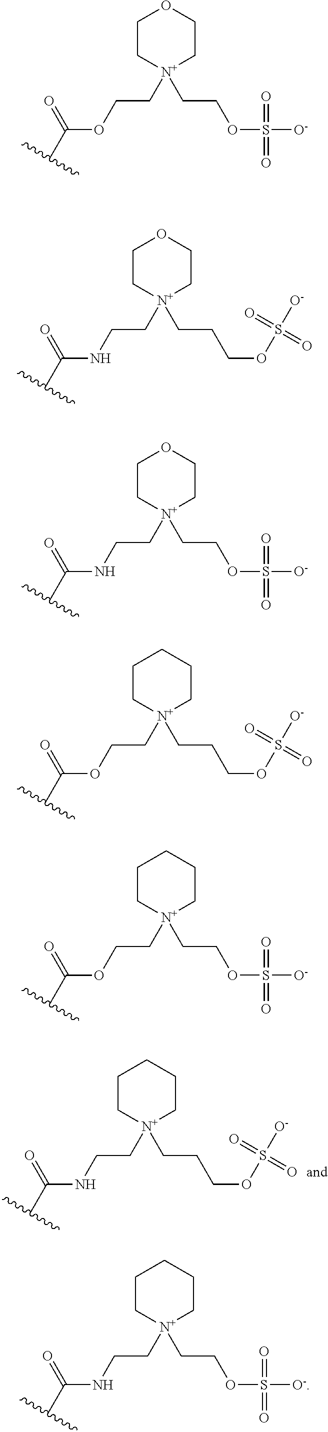

21. The method according to claim 16, wherein the polymer is selected from the group consisting of

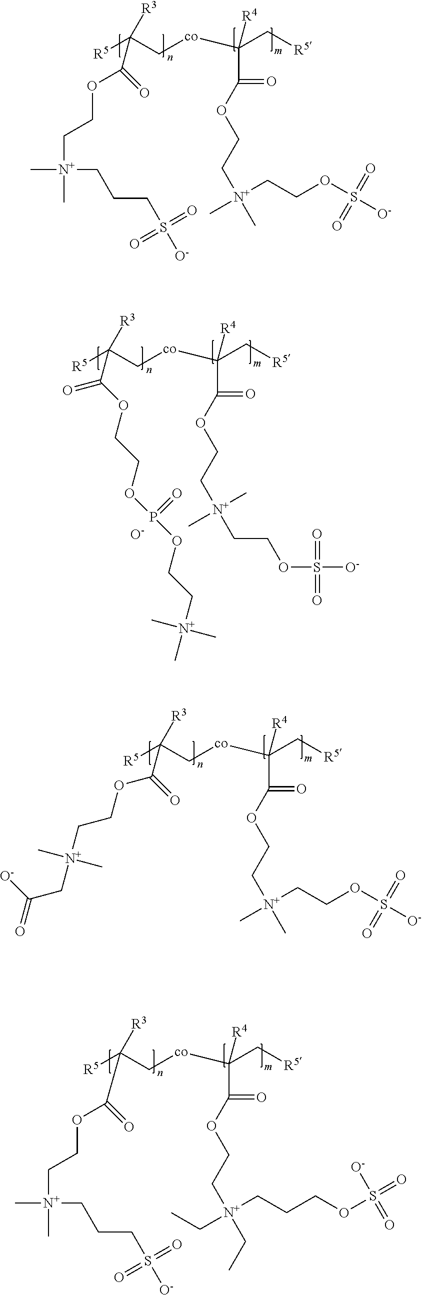

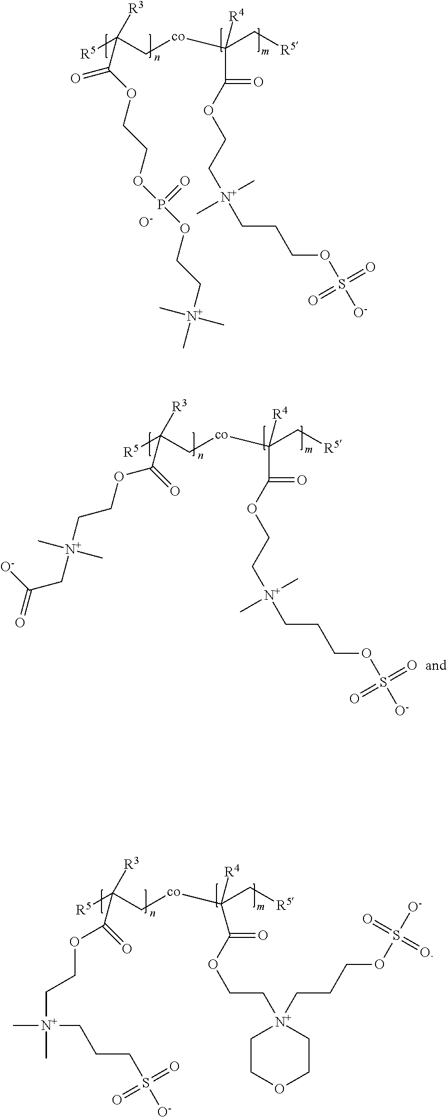

22. The method according to claim 16, wherein the crosslinker is selected from the group consisting of 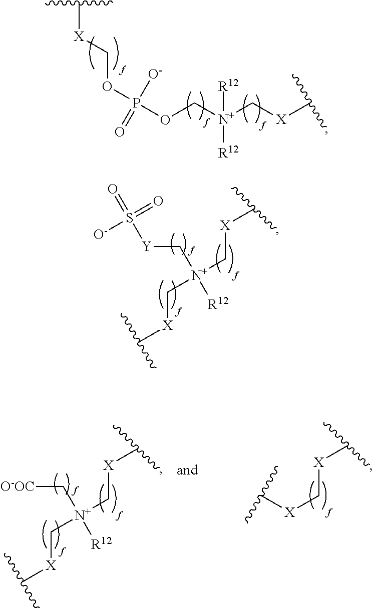
wherein
f is an integer selected from 1 to 5;
X is selected from the group consisting of —C(═O)—O—, —C(═O)—NH—, aliphatic carbocycle or heterocycle, and aromatic carbocycle or heterocycle;
Y is oxygen or absent; and
R¹² is selected from the group consisting of methyl, ethyl, and propyl.

23. The method according to claim 22, wherein the crosslinker is selected from the group consisting of

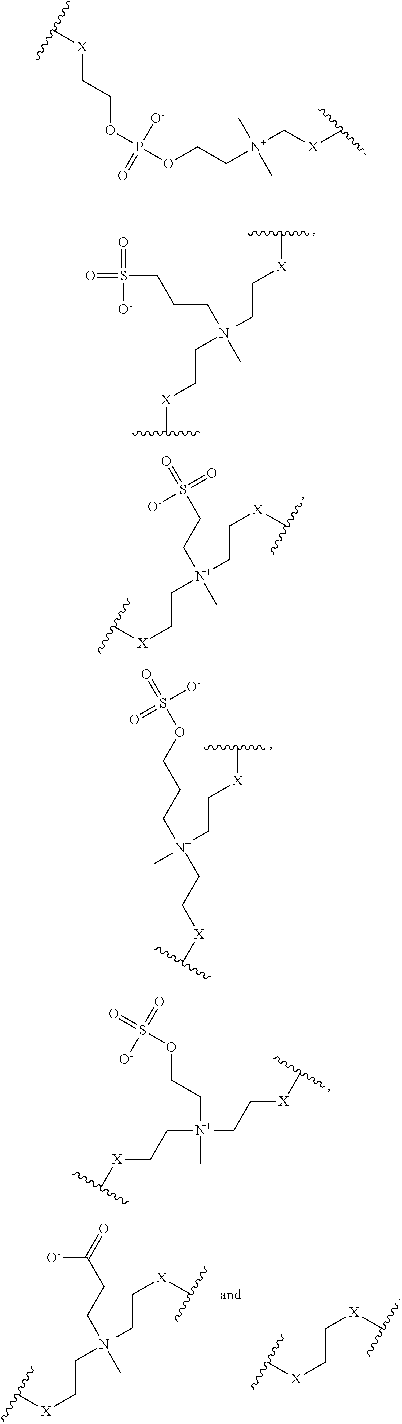

24. The method according to claim 16, wherein:
a. the polymer has a degree of polymerization (n+m) from 5 to 5000;
b. the percentage of n in the polymer ([n/(n+m)]*100) is in the range from 0 to 99.5;
c. the dispersity of the polymer is in the range of 1 to 5;
d. the crosslinker molar fraction in the polymer is in the range of 0 to 99%;
e. the droplets are responsive to temperature, shear, ionic strength, pH, a magnetic field, or a combination thereof; or
f. a combination thereof.

25. The method according to claim 16, wherein
a. the compounds for uptake, storage, reaction, or a combination thereof, are selected from the group consisting of small molecules, drugs, antibodies, antigens, RNA, nucleic acids, viruses, carbohydrates, membrane-bound vesicles, contaminants, DNA, exosomes, extracellular vesicles, cells, proteins, peptides, biomolecules, enzymes, and ab42; and/or
b. the uptake is affinity-controlled or mediated, binding-controlled or mediated, or a combination thereof, by interactions of the compound with at least one of R⁵ or R^{5′} of Formula (I).

26. The method according to claim 16, wherein the droplets are used
(i) for diagnosis of compounds including biomarkers,
(ii) for isolation, for removal, for enrichment, or a combination thereof, of compounds,
(iii) for purification, extraction, separation, or a combination thereof, of compounds,
(iv) for detoxification,
(v) for drug screening,
(vi) as cell culture scaffolds, or
(vii) in affinity assays.

27. The method according to claim 16, wherein the TBM is attached to or reacted with a group resulting from a polymerization initiator.

28. The method according to claim 16, wherein the group resulting from a polymerization initiator is a group resulting from a reversible addition-fragmentation chain transfer (RAFT) polymerization initiator.

29. The method according to claim 16, wherein the group resulting from a reversible addition-fragmentation chain transfer (RAFT) polymerization initiator is

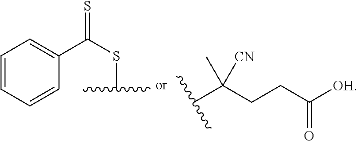

30. The method according to claim 27, wherein the TBM attached to or reacted with a group resulting from a polymerization initiator is

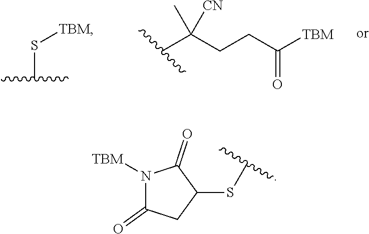

31. The method according to claim 16, wherein the TBM is selected from the group consisting of azide, alkyne, carboxylate, amine, thiol, hydroxyl, aldehyde, cyanate, sulfonyl, tosyl, tresyl, epoxide, carbonate, anhydride, carbamate, imidazole, azlactone, triazine, maleimide, aziridine, peroxide, acyl, anthraquinone, diazo, diazirine, psoralen, NHS ester, imido ester, (C₂₋₁₀)alkenyl group, (C₂₋₁₀)alkyl group, DBCO, cytosine, guanidine, streptavidin, avidin, biotin, an aptamer, a peptide, a protein, an oligonucleotide, a positively charged oligopolymer, negatively charged oligopolymer, a molecularly imprinted polymer, an affinity polymer, a nucleic acid, a carbohydrate, a dye, a cell, an antibody, an organic nanoparticle, inorganic nanoparticle, and chelating agent.

32. The method according to claim 31, wherein the peptide comprises an affinity tag, optionally an affinity tag selected from FLAG, HIS and ALFA.

33. The method according to claim 31, wherein the chelating agents are ethylenediaminetetraacetic acid (EDTA).

34. The method according to claim 16, wherein the aromatic heterocycle is selected from pyridine, pyrrole, furan and imidazole.

35. The method according to claim 16, wherein at least one of:
R⁸ and R⁹ together or two R¹³ together form an aliphatic or aromatic heterocycle, or
R⁸, R⁹ and R⁶ or R⁸, R⁹ and R⁷ together form a five or six-membered ring,
wherein one of R⁸ or R⁹ is absent if R⁸ and R⁶, R⁹ and R⁶ or R⁸ and R⁷, or R⁹ and R⁷ together form an aromatic ring.

36. The method according to claim 35, wherein at least one of:
the aromatic heterocycle is selected from the group consisting of a piperidine ring, a piperazine ring, and a morpholine ring, or
the five or six-membered ring is selected from the group consisting of a pyridine ring, a piperidine ring, a pyrrole ring, a pyrimidine ring, a pyrazole ring, an imidazole ring, a pyrazine ring, an isoxazole ring, and an oxazole ring.

37. The method according to claim 16, wherein the polymer is crosslinked by a crosslinker.

38. A polymer for forming liquid droplets in an aqueous medium, wherein the polymer is a polymer as defined in claim 16 with the proviso that
(i) R¹ is not 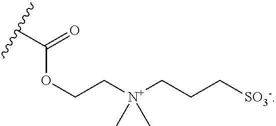
(ii) R⁵ and R^{5′} are not 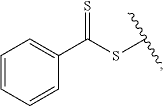
if R² is 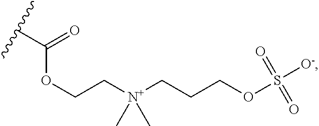
(iii) and with the proviso that R⁵ is not bromine if n=0, or
(iv) a combination thereof,
wherein the droplets take up, store, or take up and store compounds in an aqueous medium.

39. The polymer according to claim 38, wherein R¹ is selected from the group consisting of 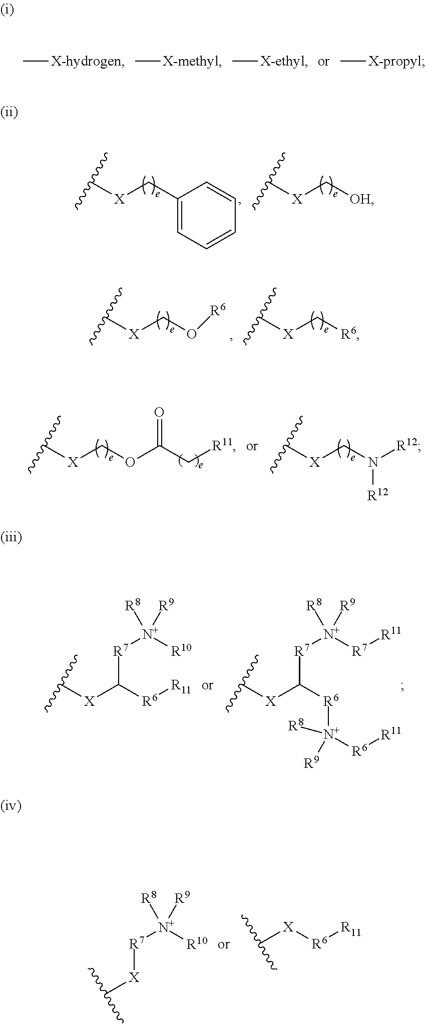
in a ratio of about 1:1 within the polymer;

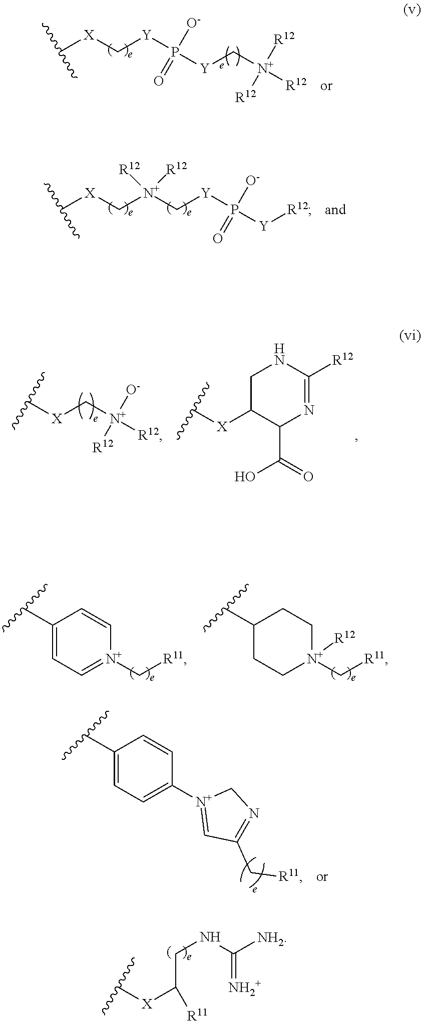

40. The polymer according to claim 38, wherein
R¹ is selected from the group consisting of 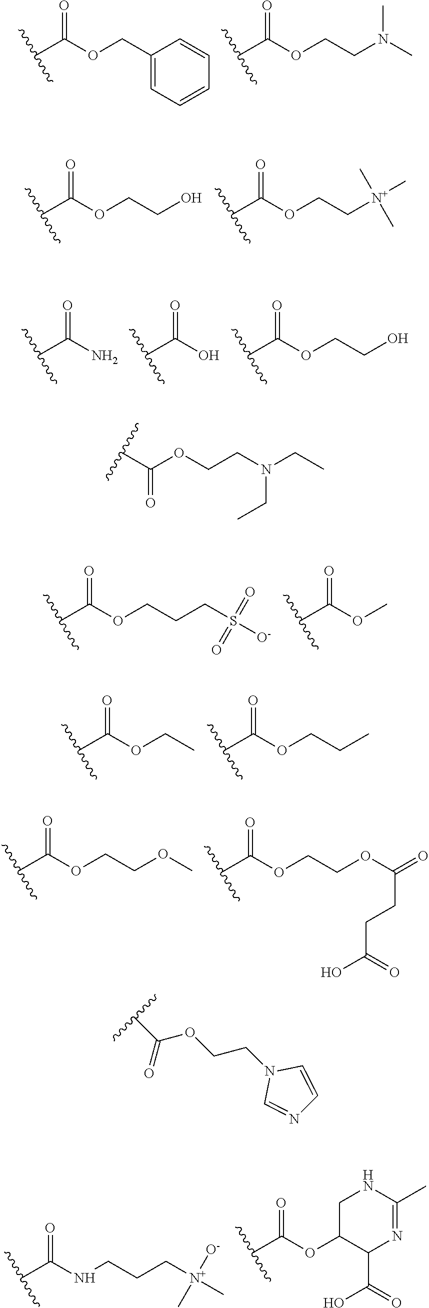 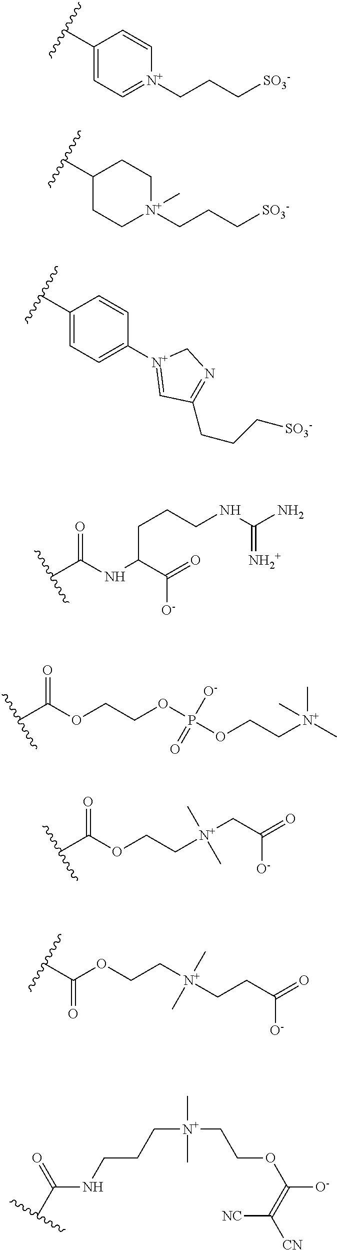 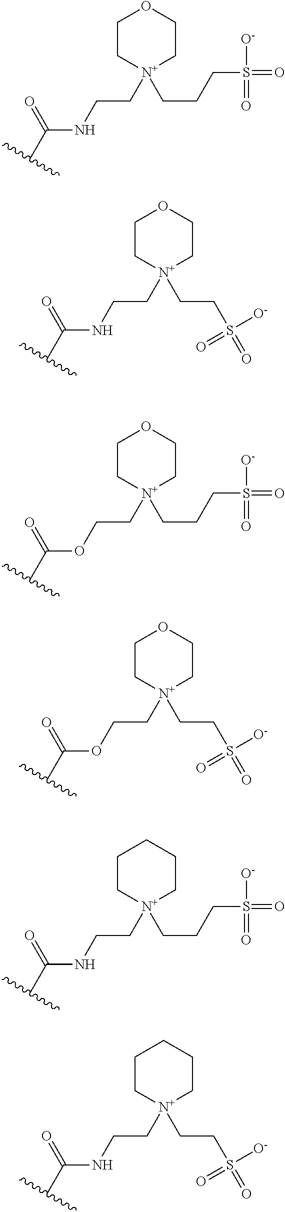 [SMILES: *C(=O)NCC[N+]1(CCCS(=O)(=O)[O-])CCOCC1 ; *C(=O)NCC[N+]1(CCS(=O)(=O)[O-])CCOCC1 ; *C(=O)OCC[N+]1(CCCS(=O)(=O)[O-])CCOCC1 ; *C(=O)OCC[N+]1(CCS(=O)(=O)[O-])CCOCC1 ; *C(=O)NCC[N+]1(CCCS(=O)(=O)[O-])CCCCC1 ; *C(=O)NCC[N+]1(CCS(=O)(=O)[O-])CCCCC1] 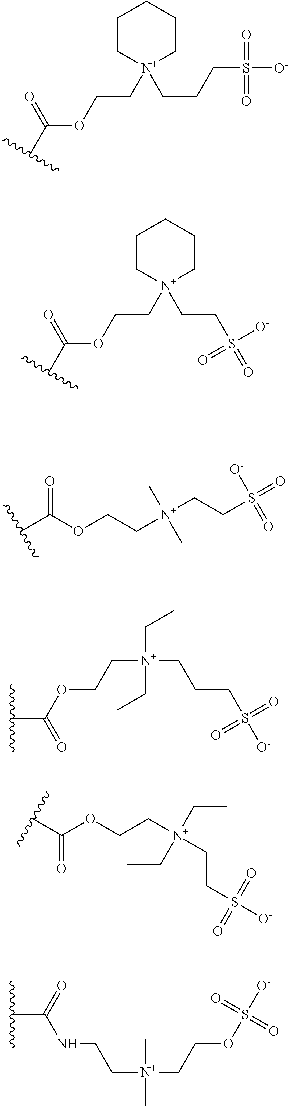 [SMILES: *C(=O)OCC[N+]1(CCCS(=O)(=O)[O-])CCCCC1 ; *C(=O)OCC[N+]1(CCS(=O)(=O)[O-])CCCCC1 ; *C(=O)OCC[N+](C)(C)CCS(=O)(=O)[O-] ; *C(=O)OCC[N+](CC)(CC)CCCS(=O)(=O)[O-] ; *C(=O)OCC[N+](CC)(CC)CCS(=O)(=O)[O-] ; *C(=O)NCC[N+](C)(C)CCOS(=O)(=O)[O-]] 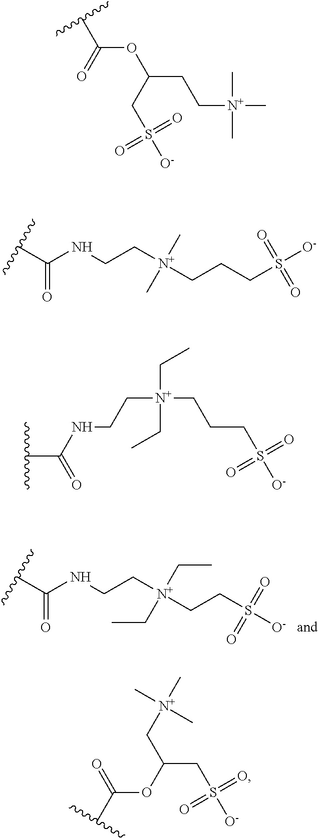 [SMILES: *C(=O)OC(CC[N+](C)(C)C)CS(=O)(=O)[O-] ; *C(=O)NCC[N+](C)(C)CCCS(=O)(=O)[O-] ; *C(=O)NCC[N+](CC)(CC)CCCS(=O)(=O)[O-] ; *C(=O)NCC[N+](CC)(CC)CCS(=O)(=O)[O-] ; *C(=O)OC(C[N+](C)(C)C)CS(=O)(=O)[O-]]
R² is selected from the group consisting of 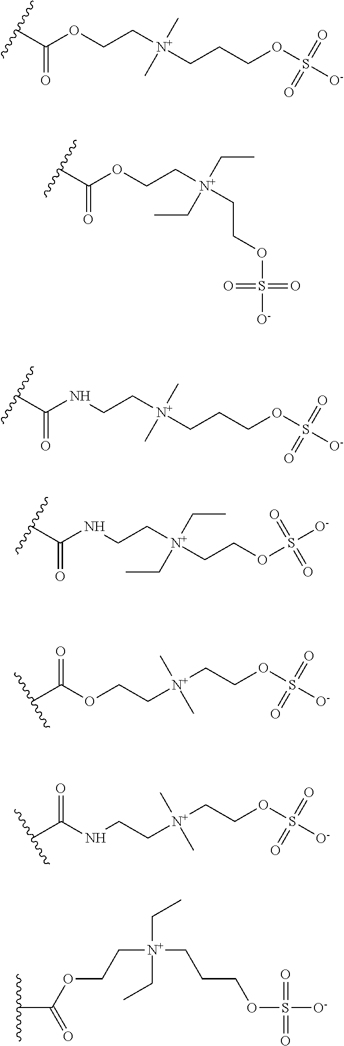 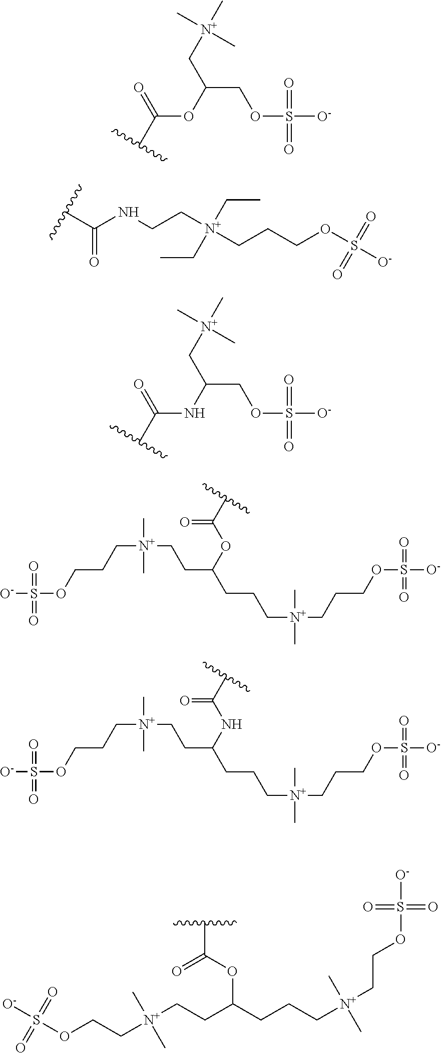 [SMILES: *C(=O)OC(COS(=O)(=O)[O-])C[N+](C)(C)C ; *C(=O)NCC[N+](CC)(CC)CCCOS(=O)(=O)[O-] ; *C(=O)NC(COS(=O)(=O)[O-])C[N+](C)(C)C ; *C(=O)OC(CCC[N+](C)(C)CCCOS(=O)(=O)[O-])CC[N+](C)(C)CCCOS(=O)(=O)[O-] ; *C(=O)NC(CCC[N+](C)(C)CCCOS(=O)(=O)[O-])CC[N+](C)(C)CCCOS(=O)(=O)[O-] ; *C(=O)OC(CCC[N+](C)(C)CCOS(=O)(=O)[O-])CC[N+](C)(C)CCOS(=O)(=O)[O-]] 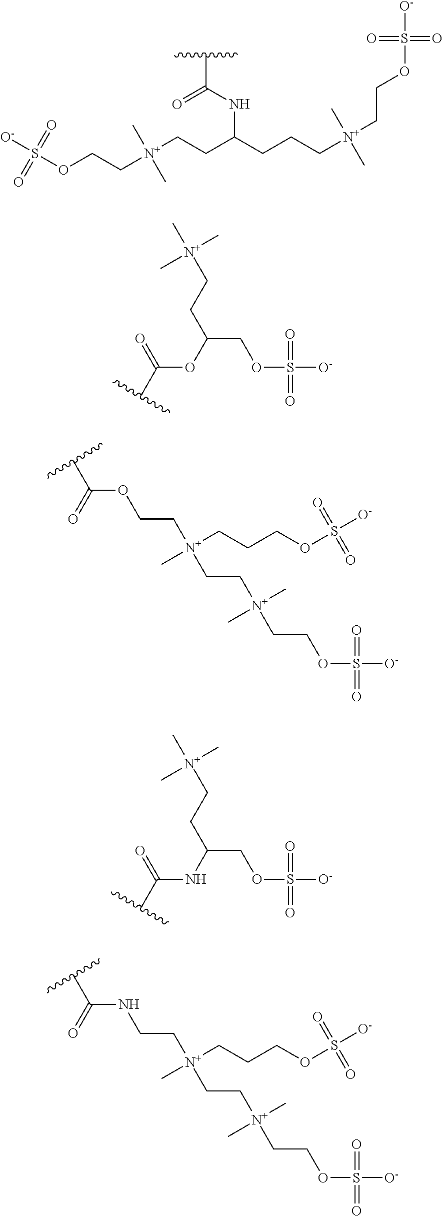 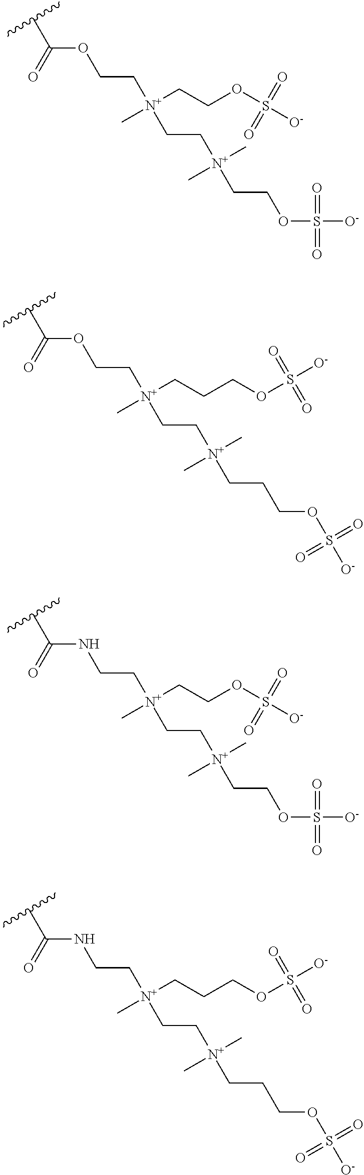 [SMILES: *C(=O)OCC[N+](C)(CCOS(=O)(=O)[O-])CC[N+](C)(C)CCOS(=O)(=O)[O-] ; *C(=O)OCC[N+](C)(CCCOS(=O)(=O)[O-])CC[N+](C)(C)CCCOS(=O)(=O)[O-] ; *C(=O)NCC[N+](C)(CCOS(=O)(=O)[O-])CC[N+](C)(C)CCOS(=O)(=O)[O-] ; *C(=O)NCC[N+](C)(CCCOS(=O)(=O)[O-])CC[N+](C)(C)CCCOS(=O)(=O)[O-]] 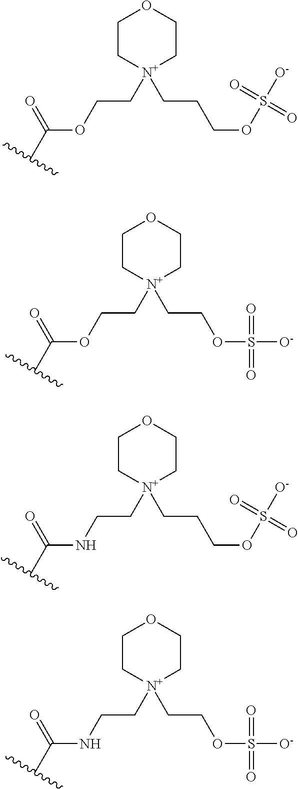 [SMILES: *C(=O)OCC[N+]1(CCCOS(=O)(=O)[O-])CCOCC1 ; *C(=O)OCC[N+]1(CCOS(=O)(=O)[O-])CCOCC1 ; *C(=O)NCC[N+]1(CCCOS(=O)(=O)[O-])CCOCC1 ; *C(=O)NCC[N+]1(CCOS(=O)(=O)[O-])CCOCC1] 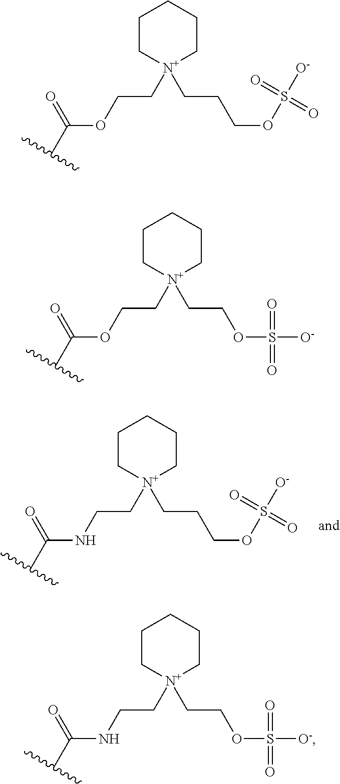
or
a combination thereof.

41. A method for producing a polymer according to claim 38 comprising the following steps:
(a) providing monomers selected from the group consisting of
methyl methacrylate, ethyl methacrylate, propyl methacrylate, butyl methacrylate, hydroxyethyl methacrylate (HEMA), methacrylamide (Mam), benzyl methacrylate (Be), 2-dimethylaminoethyl methacrylate methyl chloride (MQ) and 2-dimethylaminoethyl methacrylate (DMAEMA), 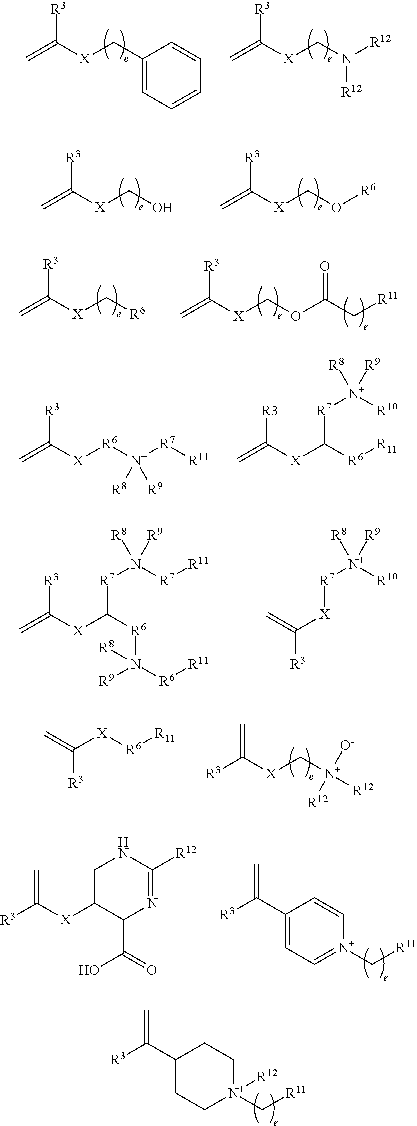 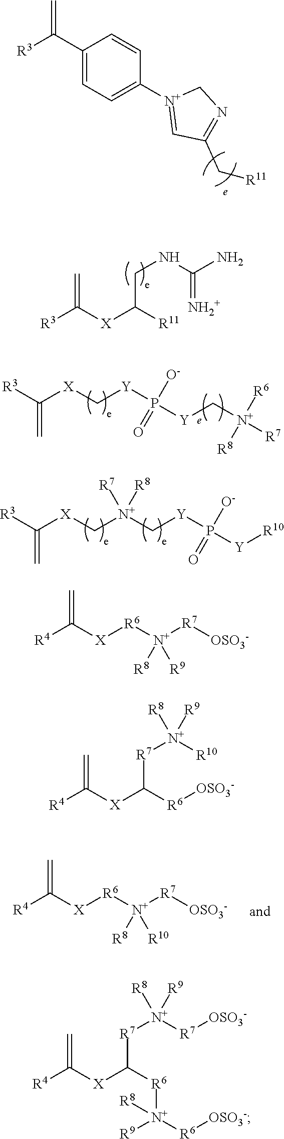
(c) reacting the monomers of step (a); and
(e) isolating and optionally purifying the polymer.

42. The method according to claim 41, further comprising step
(b) providing crosslinker monomers,
after step (a) and before step (c).

43. The method according to claim 42, wherein the crosslinker monomers are selected from the group consisting of R

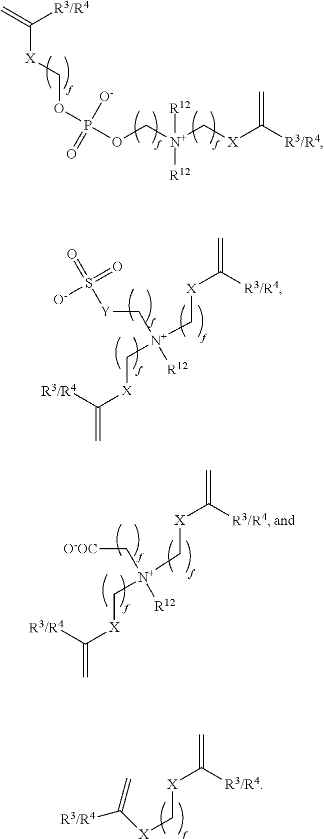

44. The method according to claim 42, wherein step (c) further comprises reacting the monomers of step (b) in a free radical polymerization or in a controlled radical polymerization.

45. The method according to claim 42, wherein the free radical polymerization or controlled radical polymerization is performed using reversible addition-fragmentation chain transfer (RAFT), atom transfer radical polymerization (ATRP), or nitroxide-mediated radical polymerization (NMP) in a suitable solvent.

46. The method according to claim 41, further comprising step
(d) functionalizing the polymer with or at R⁵ and/or R^{5′},
after step (a) and before step (e).

47. The method according to claim 46, wherein functionalizing the polymer with or at R⁵ and/or R^{5′} is performed in an esterification reaction, by click chemistry, amidation, thioesterification, or by non-covalent modification.

48. A polymer for forming liquid droplets in an aqueous medium according to claim 38.
